SEQUENCE LISTING (32 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 3 - protein, 157 aa, synthetic construct
  REGION 1..157: note = IL-18 mutant sequence
YFGKLESKLSVIRNLNDQVLFIDQGNRPLFEDMTDSDCRDNAPRTIFIISAYGDSRARGKAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 4 - protein, 193 aa, synthetic construct
  REGION 1..193: note = IL18 mutant sequence
MAAEPVEDNCINFVAMKFIDNTLYFIAEDDENLESDYFGKLESKLSVIRNLNDQVLFIDQGNRPLFEDMTDSDCRDNAPRTIFIISAYGDSRARGKAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 5 - DNA, 582 bp, Homo sapiens
  source 1..582: mol_type = other DNA
atggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 6 - DNA, 582 bp, Homo sapiens
  source 1..582: mol_type = other DNA
atggccgccgagcccgtggaggacaactgcatcaacttcgtggccatgaagttcatcgacaacacgctgtacttcatcgccgaggacgacgagaacctggagtccgactacttcggcaagctggagtccaagttgtccgtcatcaggaacttgaacgaccaggtgctcttcatcgaccagggcaaccggcccctgttcgaggacatgaccgactccgactgcagggacaacgccccccggaccatcttcatcatcagcatgtacaaggacagccagcccaggggcatggccgtgaccatctccgtgaagtgcgagaagatctccaccctctcctgcgagaacaagatcatctccttcaaggagatgaacccccccgacaacatcaaggacaccaagagcgacatcatcttcttccagaggagcgtccccggccacgacaacaagatgcagttcgagtcctcctcctacgagggctacttcctggcctgcgagaaggagagggacctgttcaagctcatcttgaagaaggaggacgagttgggggacaggtccatcatgttcaccgtgcagaacgaggactag

SEQ ID NO: 7 - DNA, 582 bp, synthetic construct
  misc_feature 1..582: note = IL18 mutant sequence
  source 1..582: mol_type = other DNA
atggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttatcatcagcgcctacggcgacagcagggccaggggcaaggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 8 - DNA, 582 bp, synthetic construct
  misc_feature 1..582: note = IL18 mutant sequence
  source 1..582: mol_type = other DNA
atggccgccgagcccgtggaggacaactgcatcaacttcgtggccatgaagttcatcgacaacacgctgtacttcatcgccgaggacgacgagaacctggagtccgactacttcggcaagctggagtccaagttgtccgtcatcaggaacttgaacgaccaggtgctcttcatcgaccagggcaaccggcccctgttcgaggacatgaccgactccgactgcagggacaacgccccccggaccatcttcatcatcagcgcctacggcgacagcagggccaggggcaaggccgtgaccatctccgtgaagtgcgagaagatctccaccctctcctgcgagaacaagatcatctccttcaaggagatgaacccccccgacaacatcaaggacaccaagagcgacatcatcttcttccagaggagcgtccccggccacgacaacaagatgcagttcgagtcctcctcctacgagggctacttcctggcctgcgagaaggagagggacctgttcaagctcatcttgaagaaggaggacgagttgggggacaggtccatcatgttcaccgtgcagaacgaggactag

SEQ ID NO: 9 - protein, 18 aa, Thoseaasigna virus 2A
EGRGSLLTCGDVEENPGP

SEQ ID NO: 10 - protein, 19 aa, Porcine teschovirus-1
ATNFSLLKQAGDVEENPGP

SEQ ID NO: 11 - protein, 20 aa, Equine rhinitis A virus
QCTNYALLKLAGDVESNPGP

SEQ ID NO: 12 - protein, 22 aa, foot and mouth disease virus
VKQTLNFDLLKLAGDVESNPGP

SEQ ID NO: 13 - skipped

SEQ ID NO: 14 - RNA, 13 nt, synthetic construct
  misc_feature 1..13: note = AU rich element
  source 1..13: mol_type = other RNA
atttatttattta

SEQ ID NO: 15-27 - skipped (13 entries)

SEQ ID NO: 28 - DNA, 282 bp, Homo sapiens
  source 1..282: mol_type = genomic DNA
taattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaa

SEQ ID NO: 29 - DNA, 282 bp, Homo sapiens
  source 1..282: mol_type = other DNA
taattaagtgcttcccacttaaaacatatcaggccttctatttatttaaatatttaaattttatatttattgttgaatgtatggtttgctacctattgtaactattattcttaatcttaaaactataaatatggatcttttatgattgaatttgtaagccctaggggctctaaaatggtttcacttatttatcccaaaatatttattattatgttgaatgttaaatatagtatctatgtagattggttagtaaaactatttaataaatttgataaatataaa

SEQ ID NO: 30 - DNA, 585 bp, Homo sapiens
  source 1..585: mol_type = genomic DNA
tggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacta

SEQ ID NO: 31 - DNA, 32 bp, synthetic construct
  misc_feature 1..32: note = Min P promoter
  source 1..32: mol_type = other DNA
tagagggtatataatggaagctcgacttccag

SEQ ID NO: 32 - DNA, 131 bp, synthetic construct
  misc_feature 1..131: note = NFAT promoter
  source 1..131: mol_type = other DNA
ggaggaaaaactgtttcatacagaaggcgtggaggaaaaactgtttcatacagaaggcgtggaggaaaaactgtttcatacagaaggcgtagatctagactctagagggtatataatggaagctcgaattc

(19) United States
(12) Patent Application Publication
(10) Pub. No.: US 2024/0425560 A1
(43) Pub. Date: Dec. 26, 2024

(54) IL-18 Variants and Uses Thereof

(71) Applicant: Chimera Bioengineering, Inc., Menlo Park, CA (US)

(72) Inventors: Benjamin Wang, Menlo Park, CA (US); Gusti Zeiner, Pacifica, CA (US)

(73) Assignee: Chimera Bioengineering, Inc., Menlo Park, CA (US)

(21) Appl. No.: 18/696,095

(22) PCT Filed: Sep. 21, 2022
(86) PCT No.: PCT/US2022/076764

§ 371 Date: Mar. 27, 2024

Related U.S. Application Data
(60) Provisional application No. 63/249,724, filed on Sep. 29, 2021.

(51) Int. Cl. C07K 14/54; C12P 21/02

(52) U.S. Cl. CPC C07K 14/54; C12P 21/02

(57) ABSTRACT

The present disclosure provides engineered IL-18 polypeptides for the production of IL-18, polynucleotides encoding the engineered IL-18, host cells expressing the engineered IL-18, and methods of using the engineered IL-18 in immunotherapy.

### REFERENCE TO SEQUENCE LISTING, TABLE OR COMPUTER PROGRAM

The official copy of the Sequence Listing is submitted concurrently with the specification as an xml file, made with WIPO Sequence Version 2.1.0, via EFS-Web, with a file name of “CBIO080.xml”, a creation date of Sep. 13, 2022, and a size of 26 kilobytes. The Sequence Listing filed via EFS-Web is part of the specification and is incorporated in its entirety by reference herein.

### BACKGROUND

Chimeric Antigen Receptors are human engineered receptors that may direct a T-cell to attack a target recognized by the CAR. For example, CAR T cell therapy has been shown to be effective at inducing complete responses against acute lymphoblastic leukemia and other B-cell-related malignancies and has been shown to be effective at achieving and sustaining remissions for refractory/relapsed acute lymphoblastic leukemia (Maude et al., NEJM, 371:1507, 2014). However, dangerous side effects related to cytokine release syndrome (CRS), tumor lysis syndrome (TLS), B-cell aplasia and on-tumor, off-target toxicities have been seen in some patients.

IL-18 is also known as interferon-gamma inducing factor as it has the ability to induce interferon-gamma production from T-cells. IL-18 is a proinflammatory cytokine that facilitates type I responses, and it modulates both innate and adaptive immunity. The activity of IL-18 is balanced by the presence of a high affinity, naturally occurring IL-18 binding protein (IL-18BP).

Interferon-gamma increases expression of IL-18BP and the production of IL-18BP acts as a negative feed-back loop.

### SUMMARY

In an aspect, the description discloses variants of IL-18 that have reduced affinity for IL-18BP. The IL-18 variants can have little or no binding interaction with IL-18BP. The IL-18 variants can have increased in vivo activity for stimulating T-cells. When the IL-18 variant and IL-12 and/or IL-15 stimulate T-cells together the induction of Tₕ₁ cells and the production of IFNg are greater for the IL-18 variants than wild-type IL-18. The combination of the IL-18 variant and IL-12 and/or IL-15 can also activate natural killer cells (NK).

In an aspect, the description discloses a eukaryotic cell with a CAR, T-cell receptor, or other targeting polypeptide and a transgene under the control of an RNA Destabilizing Element (RDE). The RDE may control multiple transgenes or multiple RDEs may control multiple transgenes. The multiple transgenes may be arranged serially and/or as a concatemer and/or in other arrangements. Multiple RDEs may be used to regulate a transgene, and these multiple RDEs can be organized as a concatemer, interspersed within a region of the transcript, or located in different parts of the transcript. Multiple transgenes can be regulated by an RDE or a combination of RDEs. The RDEs can be localized in the 3′-UTR, the 5′-UTR and/or an intron. RDEs can include, for example, the RDEs from AU 1 (CD40L), AU 2 (CSF2), AU 3 (CD247), AU 4 (CTLA4), AU 5 (EDN1), AU 6 (IL2RA), AU 7 (SLC2A1), AU 8 (TRAC), AU 9 (CD274), AU 10 (Myc), AU 11 (CD19), AU 12 (IL4), AU 13 (IL5), AU 14 (IL6), AU 15 (IL9), AU 16 (IL10), AU 17 (IL13), AU 18 (FOXP3), AU 19 (TMEM-219), AU 20 (TMEM-219snp), AU 21 (CCR7), AU 22 (SEM-A4D), AU 23 (CDC42-SE2), AU 24 (CD8), AU 27 (bGH), and/or AU 101 (Interferon gamma or IFNg). Other RDEs are disclosed in the following description. RDE control can also be combined with codon optimization of the transgene to increase the GC content of the wobble position (third position of the codon) in some or all of the codons of the transgene. This codon optimization can increase efficiency of expression (the on signal) by up to 100-fold. Such codon optimized transgenes can be linked to an RDE and produce a larger dynamic range of expression from the RDE control compared to the transgene-RDE without codon optimization.

In an aspect, the disclosure relates to CARs that are specific for tumor associated antigens. TAA and their associated cancers can include, for example, DLL3 positive cancers (such as IDH1mut gliomas, melanoma, and SCLC), CD19 positive lymphomas (e.g., NHL), onco-CD43 (sialylation mutant) positive AML, PSCA positive prostate cancer, bladder cancer or pancreatic cancer, cancer testis antigen (triple negative breast cancer), misfolded or mutant EGFR (associated with triple negative breast cancer), and/or folate receptor alpha peptide (triple negative breast cancer), SEZ6 positive small cell lung cancer (SCLC), neuroendocrine cancers (e.g., medullary thyroid cancer), large cell lung cancer (LCLC), and malignant pheochromocytoma, RNF43 positive colorectal cancer, colon cancer, and endometrial cancers, TnMUC1 positive breast cancer or pancreatic cancer, Nectin4 positive urothelial cancer, NSCLC, breast cancer, ovarian cancer, bladder cancer, pancreatic cancer, and other solid tumors, EFNA4 positive triple negative breast cancer, ovarian cancer, colorectal cancer, liver cancer, lung cancer, and other solid tumors, GPC3 positive hepatocellular carcinoma, lung cancer and other solid tumors, and Complement factor H (CFH) positive breast cancer, lung cancer, nonsmall cell lung cancer (NSCLC), small cell lung cancer (SCLC), and other solid tumors.

In an aspect, activation of the immune cell induces expression of the transgene that can encode a payload to be delivered at the target (activation) site. The transgene can encode a payload for delivery at the site of CAR activation and/or immune cell activation and/or other receptor activation. The payload can be a cytokine, an antibody, a reporter (e.g., for imaging), a receptor (such as a CAR), or other polypeptide that can have a desired effect at the target site. The payload can remain in the cell, or on the cell surface to modify the behavior of the cell. The payload can be an intracellular protein such as a kinase, phosphatase, metabolic enzyme, an epigenetic modifying enzyme, a gene editing enzyme, etc. The payload can be a gene regulatory RNA, such as, for example, siRNA, microRNAs (e.g., miR155), shRNA, antisense RNA, ribozymes, and the like, or guide RNAs for use with CRISPR systems. The payload can be a nucleic acid (e.g., a vector, or a human artificial chromosome (HAC)). The payload can also be a membrane bound protein such as GPCR, a transporter, etc. The payload can be an imaging agent that allows a target site to be imaged (target site has a desired amount of target antigen bound by the CAR). The payload can be a checkpoint inhibitor, and the CAR and/or other binding protein (e.g., T-cell receptor, antibody or innate immunity receptor) can recognize a tumor associated antigen so the eukaryotic cell preferentially delivers the checkpoint inhibitor at a tumor. The payload can be a cytotoxic compound including, for example, a granzyme, an apoptosis inducer, a cytotoxic small molecule, or complement. The payload can be an antibody, such as for example, an anti-4-1BB agonist antibody (an anti-CD137 antibody), an anti-IL 1b antibody (anti-inflammatory), anti-CD29/anti-VEGF antibody, an anti-CTLA4 antibody, a bispecific antibody (e.g., BiTE), or an anti-CD11b antibody. The payload can be an immune polypeptide, including for example, IL-18 or an IL-18 variant, other cytokines (e.g., IL-2, IL-12, IL-15), chemokines (e.g., CXCL12), perforins, granzymes, and other immune polypeptides. The payload can be an enzyme including for example, hyaluronidase, or heparinase. The payload can be a polypeptide including for example, ApoE (e.g., ApoE2, ApoE3 and ApoE4), NO synthase (e.g., iNOS, nNOS, eNOS), HSV-thymidine kinase (HSV-TK), antagonists of CSF1 receptor, CCR2, CCR4, a BiTE (activates immunosuppressed T-cells), soluble CD40 ligand, HSP70, and HSP60. The payload can be fused or associated with Decorin, Biglycan, fibromodulaon/Lumican so that the payload binds to the collagen near or in the target site. This strategy is particularly useful for keeping cytotoxic payloads localized to the target cells (e.g., a tumor). The payload can be a transgene(s) which delivers a virus as a payload. For example, the RDE can control a master control element that controls the expression of the virus genes for replication and coat/envelope proteins. Alternatively, the Rep and coat/envelope proteins can be placed under the control of inducible promoters that are controlled by a regulatory protein, and that regulatory protein can be controlled by an RDE. Still alternatively, the Rep proteins of the virus can be placed under the control of an RDE, and/or the coat/envelope proteins of the virus can be placed under the control of an RDE. As with other payloads this complex payload can use CAR T-cell regulation or any other regulation that induces glycolysis in a cell. Helper constructs in a T cell, or other delivery cell can encode the genes needed for viral replication and viral packaging.

In an aspect, a therapy utilizing a CAR T-lymphocyte with or without an RDE controlled transgene(s) is combined or in an order of succession with another therapy. The other therapy can include any therapeutic molecule including, for example, a polypeptide, lipid, carbohydrate, nucleic acid, small molecule drug, biological drug, antibody, antibody-drug-conjugate, or combinations of the foregoing. Suitable molecules are described below. The other therapy can be administered to a subject at the same time as the CAR therapy (with or without a RDE controlled transgene(s)), before the administration of the CAR therapy (with or without a RDE controlled transgene(s)), or after the administration of the CAR therapy (with or without a RDA controlled transgene(s)). For example, a subject could be treated with chemotherapy and/or an immunotherapy (e.g., an antibody-drug conjugate), followed by treatment with a CAR T-cell with optionally a RDE controlled payload. The CAR T-cell treatment can be given the subject at varying times after the chemotherapy and/or immunotherapy, e.g., one, two, three, four, five, or six weeks. The chemotherapy and/or immunotherapy (e.g., ADC) can be cycled with the CAR T-cell treatment for multiple cycles of treatment. Treatment with CAR T-cells may also be boosted with target X peptide, or virus or cells loaded with target X peptide (target X is the target bound by the CAR).

In an aspect, the description discloses methods, cells and nucleic acids for reducing the inhibition of T-cells in certain tumor microenvironments (TME). Notch receptor signaling on T-cells during activation of the T-cell can suppress the activation and proliferation of the T-cell. Many tumor environments have cells that can express Notch receptor ligands such as, for example, DLL1, DLL4, Jagged1 and Jagged2, and these Notch ligands can induce Notch signaling in T-cells that suppresses activation and proliferation of the T-cell. This suppression of T-cell activation can be reduced using inhibitors of Notch receptor signaling including, for example, dominant negative Notch receptor components or Notch signal processing components in the cell, Notch receptor antagonists, gamma secretase inhibitors, and/or ADAM protease inhibitors (e.g., ADAM17 inhibitors).

### BRIEF DESCRIPTION OF THE FIGURES

FIG. 1A-D shows graphs for activation of T-cells transduced with IL-18, an IL-18 variant, or non-transduced. FIGS. 1A and B show activation after 24 hours. FIGS. 1C and D show activation after 72 hours.

FIG. 2 shows a graph for the bioluminescence from T-cells with luciferase controlled by an RDE following activation of the T-cell by Raji target cells (activate CAR) or by CD3/CD28 beads (activate TCR) as compared to bioluminescence of T-cells at resting.

FIG. 3 shows a graph for bioluminescence from T-cells with luciferase controlled by the RDEs Gold1, Gold2, or Gold3 following activation of the T-cell by Raji target cells (activate CAR) as compared to bioluminescence of T-cells at resting.

FIG. 4 shows a graph for the IL-12 expression from T-cells with IL-12 expression controlled by an RDE following activation of the T-cell by Raji target cells (activate CAR) as compared to IL-12 expression of T-cells at resting.

FIG. 5 shows basal luciferase expression and activated luciferase expression for luciferase constructs utilizing different RDEs as control elements in Jurkat cells.

FIG. 6 shows basal luciferase expression and activated luciferase expression for luciferase constructs utilizing different RDEs as control elements in primary T-cells.

FIG. 7 shows activated luciferase/basal luciferase expression after 1, 3, 6, and 8 days for luciferase constructs utilizing different RDEs as control elements.

FIG. 8 shows basal luciferase expression and activated luciferase expression for luciferase constructs utilizing different RDEs as control elements.

FIG. 9 shows the dynamic range (activated luciferase/basal luciferase) measured 1, 3/4, 6, and 8 days after activation for luciferase constructs utilizing different RDEs as control elements.

FIG. 10 shows the dynamic range (activated luciferase/basal luciferase) measured 1, 3/4, 6, and 8 days after activation for luciferase constructs utilizing different RDEs as control elements.

FIG. 11 shows the impact on luciferase expression for luciferase constructs utilizing an RDE as a control element in the presence of glucose and galactose.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

As used in this specification and the appended claims, the singular forms “a” “an” and “the” include plural referents unless the context clearly indicates otherwise. Thus, for example, reference to “a polypeptide” includes more than one polypeptide.

The section headings used herein are for organizational purposes only and not to be construed as limiting the subject matter described.

### Definitions

As used herein, the terms “amino acid substitution” or “amino acid difference” are defined to mean a change in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. In most instances herein, the specific amino acid substitution or amino acid residue difference at a position is indicated as “XnY” where “Xn” specifies the corresponding position as described above, and “Y” is the single letter identifier of the amino acid found in the engineered polypeptide (i.e., the different residue than in the reference polypeptide). More than one amino acid can appear at a specified residue position, the alternative amino acids can be listed in the form XnY/Z, where Y and Z represent alternate amino acid residues. In some instances, the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Furthermore, in some instances, a polypeptide of the present disclosure can include one or more amino acid residue differences relative to a reference sequence, which is indicated by a list of the specified positions where changes are made relative to the reference sequence.

As used herein, the term “chromosomal integration” is defined to mean the process whereby an incoming sequence is introduced into the chromosome of a host cell. The homologous regions of the transforming DNA align with homologous regions of the chromosome. Subsequently, the sequence between the homology boxes is replaced by the incoming sequence in a double crossover (i.e., homologous recombination). Homologous sections of an inactivating chromosomal segment of a DNA construct may align with the flanking homologous regions of the indigenous chromosomal region of a host cell chromosome. Subsequently, the indigenous chromosomal region is deleted by the DNA construct in a double crossover (i.e., homologous recombination).

As used herein, the term “coding sequence” is defined to mean a portion of a nucleic acid (e.g., a gene) that encodes an amino acid sequence of a protein.

As used herein, the term “codon optimized” is defined to mean changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. The polynucleotides encoding the IL-18 may be codon optimized for optimal production from the host organism selected for expression.

As used herein, the terms “consensus sequence” and “canonical sequence” are defined to mean an archetypical amino acid sequence against which all variants of a particular protein or sequence of interest are compared. The terms also refer to a sequence that sets forth the nucleotides that are most often present in a DNA sequence of interest. For each position of a gene, the consensus sequence gives the amino acid that is most abundant in that position in a multiple sequence alignment (MSA).

As used herein, the terms “conservative amino acid substitution” or “conservative amino acid difference” are defined to mean a change in the amino acid at a residue position to a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

Exemplary conservative substitutions are provided in Table 1 below.

TABLE 1

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

As used herein, the term “control sequence” is defined to include all components, which are necessary or advantageous for the expression of a polynucleotide and/or polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and where appropriate, translational stop signals. The control sequences may be provided with linkers for the purpose of introducing specific restriction sites facilitating ligation of the control sequences with the coding region of the nucleic acid sequence encoding a polypeptide.

As used herein, the terms “corresponding to”, “reference to” or “relative to” are used interchangeably when used in the context of the numbering of a given amino acid or polynucleotide sequence and are defined in this context to mean the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered IL-18, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. As such, the term “corresponding to”, “reference to” or “relative to” also refers to a residue that is analogous, homologous, or equivalent to an enumerated residue in a reference polypeptide. In addition, crystal structure coordinates of a reference sequence may be used as an aid in determining a homologous polypeptide residue's three dimensional structure and location of equivalent residues.

As used herein, the term “deletion” is defined to mean a modification of a polypeptide by removal of one or more amino acids from the reference polypeptide or modification of a nucleic acid by removal of one or more nucleotides from the reference nucleic acid. For example, deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining enzymatic activity and/or retaining the improved properties of an engineered IL-18. Deletions can be directed to the internal portions and/or terminal portions of the polypeptide. The deletion can comprise a continuous segment or can be discontinuous.

As used herein, the term “gene” is defined to mean a polynucleotide (e.g., a DNA segment) that encodes a polypeptide. The term may include regions preceding and following the coding regions as well as any intervening sequences when present (e.g., introns) between individual coding segments (exons).

As defined herein, the term “heterologous” polynucleotide or polypeptide is defined to mean any polynucleotide or polypeptide that is not naturally found in a host cell. As such, the term includes polynucleotides that are removed from a host cell, subjected to laboratory manipulation, and then reintroduced into a host cell. The introduced polynucleotide can express the heterologous polypeptide.

As used herein, the term “homologous genes” is defined to mean a pair of genes which correspond to each other and which are identical or similar to each other. The term encompasses genes that are separated by speciation (i.e., the development of new species) (e.g., orthologous genes), as well as genes that have been separated by genetic duplication (e.g., paralogous genes).

As used herein, the term “homologous recombination” is defined to mean the exchange of DNA fragments between two DNA molecules or paired chromosomes at the site of identical or nearly identical nucleotide sequences. Chromosomal integration can be homologous recombination.

As used herein, the term “improved IL-18 property” is defined to mean a IL-18 property that exhibits an improvement as compared to a reference IL-18 polypeptide. For the engineered IL-18 polypeptides described herein, the comparison is generally made to the naturally occurring IL-18, although the reference IL-18 polypeptide can be another engineered IL-18.

Any property relating to IL-18 activity may be affected, including any pharmacodynamic or pharmacokinetic property such as serum half-life, area under the curve, Tₘₐₓ, Cₘₐₓ, etc.

As used herein, the term “insertion” is defined to mean a modification to a polypeptide by addition of one or more amino acids from the reference polypeptide, or modification of a nucleic acid by addition of one or more nucleic acids. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the reference polypeptide.

As used herein, the term “isolated polypeptide” is defined to mean a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis).

As used herein, the terms “microbial,” “microbial organism” or “microorganism” are defined to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the terms “non-conservative substitution” or “non-conservative amino acid difference” are defined to mean a change in the amino acid at a residue position to a different residue with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, the term “operably linked” is defined to mean a configuration in which a control sequence is appropriately placed (i.e., in a functional relationship) at a position relative to a polynucleotide of interest such that the control sequence directs or regulates the expression of the polynucleotide and/or polypeptide of interest.

As used herein, the terms “optimal alignment” or “optimally aligned” are defined to mean the alignment of two (or more) sequences giving the highest percent identity score. For example, optimal alignment of two polypeptide sequences can be achieved by aligning the sequences such that the maximum number of identical amino acid residues in each sequence are aligned together or by using software programs or procedures described herein or known in the art. Optimal alignment of two nucleic acid sequences can be achieved by aligning the sequences such that the maximum number of identical nucleotide residues in each sequence are aligned together. Two sequences (e.g., polypeptide sequences) may be deemed “optimally aligned” when they are aligned using defined parameters, such as a defined amino acid substitution matrix, gap existence penalty (also termed gap open penalty), and gap extension penalty, so as to achieve the highest similarity score possible for that pair of sequences. Optimal alignment can be done manually or by using software programs or procedures described herein or known in the art. e.g., the BLASTP program for amino acid sequences and the BLASTN program for nucleic acid sequences.

As used herein, the terms “ortholog” and “orthologous genes” are defined to mean genes in different species that have evolved from a common ancestral gene (i.e., a homologous gene) by speciation. Typically, orthologs retain the same function during the course of evolution.

Identification of orthologs finds use in the reliable prediction of gene function in newly sequenced genomes.

As used herein, the terms “paralog” and “paralogous genes” are defined to mean genes that are related by duplication within a genome. Generally, paralogs tend to evolve into new functions, even though some functions are often related to the original one.

As used herein, the terms “percentage of sequence identity” and “percentage homology” are used interchangeably and are defined to mean comparisons among polynucleotides or polypeptides, and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv Appl Math. 2:482, 1981; by the homology alignment algorithm of Needleman and Wunsch, J Mol Biol. 48:443, 1970; by the search for similarity method of Pearson and Lipman, Proc Natl Acad Sci. USA 85:2444, 1988; by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for determining percent sequence identity and sequence similarity are the BLAST and BLAST 2.0 algorithms, which are described in Altschul et al., J. Mol. Biol. 215:403-410, 1990; and Altschul et al., Nucleic Acids Res. 25(17):3389-3402, 1977; respectively. Software for performing BLAST analyses is publicly available through the National Center for Biotechnology Information website. BLAST for nucleotide sequences can use the BLASTN program with default parameters, e.g., a wordlength (W) of 11, an expectation (E) of 10, M=5, N=−4, and a comparison of both strands. BLAST for amino acid sequences can use the BLASTP program with default parameters, e.g., a wordlength (W) of 3, an expectation (E) of 10, and the BLOSUM62 scoring matrix (see Henikoff and Henikoff, Proc Nat Acad Sci. USA 89:10915, 1989). Exemplary determination of sequence alignment and % sequence identity can also employ the BESTFIT or GAP programs in the GCG Wisconsin Software package (Accelrys, Madison WI), using default parameters provided.

As used herein, the terms “polynucleotide” or “nucleic acid’ are used interchangeably and are defined to mean two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. Preferably, such modified or synthetic nucleobases will be encoding nucleobases.

As used herein, the term “promoter sequence” is defined to mean a nucleic acid sequence that is recognized by a host cell for expression of a polynucleotide of interest, such as a coding sequence or gene. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

As used herein, the terms “protein”, “polypeptide,” and “peptide” are used interchangeably and are defined to mean a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids. The standard single or three letter abbreviations can be used for the genetically encoded amino acids (see, e.g., IUPAC-IUB Joint Commission on Biochemical Nomenclature, “Nomenclature and Symbolism for Amino Acids and Peptides,” Eur. J. Biochem. 138:9-37, 1984).

As used herein, the terms “recombinant” or “engineered” or “non-naturally occurring” are used interchangeably and are defined to mean modified polypeptides or nucleic acids which polypeptides or nucleic acids are modified in a manner that would not otherwise exist in nature, or is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein, the term “reference sequence” is defined to mean a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. A “reference sequence” can be based on a primary amino acid sequence, where the reference sequence is a sequence that can have one or more changes to the primary sequence.

As used herein, the term “stringent hybridization conditions” is defined to mean hybridizing in 50% formamide at 5×SSC at a temperature of 42° C. and washing the filters in 0.2×SSC at 60° C. (1×SSC is 0.15M NaCl, 0.015M sodium citrate.) Stringent hybridization conditions also encompasses low ionic strength and high temperature for washing, for example 0.015 M sodium chloride/0.0015 M sodium citrate/0.1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As used herein, the term “substantial identity” refers to a polynucleotide or polypeptide sequence that has at least 80 percent sequence identity, at least 85 percent identity and 89 to 95 percent sequence identity, more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. The term “substantial identity” can mean that two polypeptide sequences, when optimally aligned, such as by the programs GAP or BESTFIT using standard parameters, i.e., default parameters, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

As used herein, the term “substantially pure polypeptide” is defined to mean a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure IL-18 composition will comprise about 60% or more, about 70% or more, about 80% or more, about 90% or more, about 95% or more, and about 98% or more of all macromolecular species by mole or % weight present in the composition. The object species can be purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. The isolated engineered IL-18 polypeptide can be a substantially pure polypeptide composition.

As used herein, the terms “wild-type” is defined to mean the form found predominantly in nature. For example, a wild-type polypeptide or polynucleotide sequence is a sequence predominantly present in an organism that can be isolated from a source in nature and which has not been intentionally modified by human manipulation.

### Variant IL-18

The disclosure provides polypeptides having IL-18 activity, polynucleotides encoding these polypeptides, host cells containing the polynucleotides, and methods for using the polypeptides and host cells for the variant IL-18 polypeptides. Where the description relates to polypeptides, it is to be understood that it also describes the polynucleotides encoding the polypeptides.

Interleukin-18 (IL-18) is a member of the IL-1 family of cytokines. IL-18 is a proinflammatory cytokine that facilitates type I responses, and it modulates both innate and adaptive immunity. Together with IL-12, IL-18 participates in the Th1 paradigm. This property of IL-18 is due to its ability to induce IFNγ either with IL-12 or IL-15. Interleukin-18 exhibits characteristics of other pro-inflammatory cytokines, such as increases in cell adhesion molecules, nitric oxide synthesis, and chemokine production. The activity of IL-18 is balanced by the presence of a high affinity, naturally occurring IL-18 binding protein (IL-18BP). Interferon-gamma increases expression of IL-18BP and the production of IL-18BP acts as a negative feed-back loop.

The disclosure provides engineered IL-18 having improved in vivo activity, for example, activation of T-cells to produce IFNg. Variants were made by changing specific codons of the encoding nucleic acid. Codons were selected for change that were in the portion of IL-18 which interacts with IL-18BP. Variants were screened for loss of binding to IL-18BP and increased in vivo activity (activating T-cells). The IL-18 variants had one or more of the following changes in SEQ ID NO: 1, M51A, K53G, Q56R, P57A and/or M60K (numbering is for the mature, active IL-18, after secretion expression and caspase-1 processing). Alternative numbering from the full-length, unprocessed protein of SEQ ID NO: 2 are M87A, K89G, Q92R, P93A and/or M96K.

(SEQ ID NO: 1)
YFGK LESKLSVIRN LNDQVLFIDQ GNRPLFEDMT DSDCRDNAPR
TIFIISMYKD SQPRGMAVTI SVKCEKISTL SCENKIISFK
EMNPPDNIKD TKSDIIFFQR SVPGHDNKMQ FESSSYEGYF
LACEKERDLF KLILKKEDEL GDRSIMFTVQ NED
(SEQ ID NO: 2)
MAAEPVEDNC INFVAMKFID NTLYFIAEDD ENLESD YFGK
LESKLSVIRN LNDQVLFIDQ GNRPLFEDMT DSDCRDNAPR
TIFIISMYKD SQPRGMAVTI SVKCEKISTL SCENKIISFK
EMNPPDNIKD TKSDIIFFQR SVPGHDNKMQ FESSSYEGYF
LACEKERDLF KLILKKEDEL GDRSIMFTVQ NED

The amino acids in bold indicate some positions that can be changed in the IL-18 variants.

Two variants are found in SEQ ID NO: 3 and 4:

(SEQ ID NO: 3)
YFGK LESKLSVIRN LNDQVLFIDQ GNRPLFEDMT DSDCRDNAPR
TIFIISAYGD SRARGKAVTI SVKCEKISTL SCENKIISFK
EMNPPDNIKD TKSDIIFFQR SVPGHDNKMQ FESSSYEGYF
LACEKERDLF KLILKKEDEL GDRSIMFTVQ NED
(SEQ ID NO: 4)
MAAEPVEDNC INFVAMKFID NTLYFIAEDD ENLESD YFGK
LESKLSVIRN LNDQVLFIDQ GNRPLFEDMT DSDCRDNAPR
TIFIISAYGD SRARGKAVTI SVKCEKISTL SCENKIISFK
EMNPPDNIKD TKSDIIFFQR SVPGHDNKMQ FESSSYEGYF
LACEKERDLF KLILKKEDEL GDRSIMFTVQ NED

The amino acids in bold indicate some positions that can be changed in the IL-18 variants.

Accordingly, the present disclosure can provides engineered human IL-18 polypeptide capable of reduced interaction with IL-18BP with improved in vivo activity for stimulating T-cells. The reference IL-18 can corresponds to an amino acid sequence of SEQ ID NO: 1 or 2 from humans. It is to be understood that various orthologs and paralogs, including orthologs and paralogs of SEQ ID NO:1 or 2, can be used.

The engineered IL-18 variants can have reduced binding for TL-18 binding protein as compared to a reference polypeptide, e.g., SEQ ID NO: 1 or 2. The engineered IL-18 polypeptide can have increased in vivo activity for T-cells as compared to the reference polypeptide, e.g., SEQ ID NO: 1 or 2.

The advantageous properties of the engineered IL-18 variants disclosed herein can be associated with amino acid substitutions at residue positions corresponding to M51, K53, Q56, P57 and/or M60 (SEQ ID NO: 1) or M87, K89, Q92, P93 and/or M96 (SEQ ID NO: 2), where the amino acid substitutions and residue positions are with respect to the reference sequence of SEQ ID NO: 1 or 2. Without being bound by theory, amino acid substitutions at the foregoing residue positions affect binding with IL-18 binding protein. While the residue positions correspond to the reference sequence of SEQ ID NO: 1 or 2, it is to be understood that equivalent residue positions can be identified in other reference enzymes that has structural similarity to the reference sequence of SEQ ID NO: 1 or 2, including various orthologs and paralogs. The equivalent positions are readily determined by alignment of a target sequence with the reference sequence using sequence alignment software, particularly using optimal alignment of the target sequence with the reference sequence as described herein and as is well known in the art.

The engineered IL-18 variant can have at least 60%, 65%, 70%, 75%, 80%, 85%, 90%, 91%, 92%, 93%, 94%, 95%, 96%, 97%, 98%, 99% or more amino acid sequence identity to the reference sequence of SEQ ID NO: 1 or 2 and having at least two amino acid substations at residue positions corresponding to: M51, K53, Q56, P57 and/or M60 (SEQ ID NO: 1) or M87, K89, Q92, P93 and/or M96 (SEQ ID NO: 2).

The engineered IL-18 variant can have at least 60%, 65%, 70%, 75%, 80%, 85%, 90%, 91%, 92%, 93%, 94%, 95%, 96%, 97%, 98%, 99% or more amino acid sequence identity to the reference sequence of SEQ ID NO: 1 or 2 and having at least amino acid substitutions at residue positions: M51, K53, Q56, P57 and/or M60 (SEQ ID NO: 1) or M87, K89, Q92, P93 and/or M96 (SEQ ID NO: 2).

The engineered IL-18 variant described herein can have the following amino acid changes at residue positions:
M51/M87 is selected from A, S, and G;
K53/K89 is selected from G, A, and S;
Q56/Q92 is selected from R, K and H;
P57/P93 is selected from A, S, and G;
M60/M96 is selected from R, K, and H;

The engineered IL-18 variant has at least 60%, 65%, 70%, 75%, 80%, 85%, 90%, 91%, 92%, 93%, 94%, 95%, 96%, 97%, 98%, 99% or more amino acid sequence identity to the reference sequence of SEQ ID NO: 1 or 2 and at least three of the following amino acid substitutions: M51, K53, Q56, P57 and/or M60 (SEQ ID NO: 1) or M87, K89, Q92, P93 and/or M96 (SEQ ID NO: 2)

The engineered IL-18 variants can be in various forms, for example, such as an isolated preparation, as a substantially purified preparation, whole cells transformed with gene(s) encoding the polypeptide, and/or as cell extracts and/or lysates of such cells. The IL-18 variants can be lyophilized, spray-dried, precipitated or be in the form of a crude paste, as further discussed below. Any of the engineered IL-18 variants expressed in a host cell can be recovered from the cells and or the culture medium using any one or more of the well known techniques for protein purification, including, among others, lysozyme treatment, sonication, filtration, salting-out, ultra-centrifugation, and chromatography.

Chromatographic techniques for isolation of the IL-18 variants include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

Affinity techniques may be used to isolate the engineered IL-18 variants. Affinity chromatography purification can be done with any antibody which specifically binds the IL-18 variant may be used (e.g., antibodies binding to epitopes separate from the IL-18BP site). For the production of antibodies, various host animals, including but not limited to rabbits, mice, rats, etc., may be immunized by injection with a IL-18 variant, or a fragment thereof. A variety of well-known anti-IL-18 antibodies can also be used, for example, commercially sold anti-IL-18 antibodies can be used. The affinity purification can also use a IL-18 receptor.

### Polynucleotides and Expression Vectors

In another aspect, polynucleotides can encode any of the engineered IL-18 variants described herein. Exemplary nucleotides are found at SEQ ID NO: 5-8. SEQ ID NO: 5 encodes the wild-type human IL-18 sequence. SEQ ID NO: 6 encodes the wild-type human IL-18 sequence that has been GC3 optimized to increase GC3 content. SEQ ID NO: 7 encodes an IL-18 variant with M87, K89, Q92, P93 and/or M96 (SEQ ID NO: 2). SEQ ID NO: 8 encodes an IL-18 variant with M87, K89, Q92, P93 and/or M96 (SEQ ID NO: 2) that has modified to increase GC3 content.

(SEQ ID NO: 5)
ATGGCTGCTG AACCAGTAGA AGACAATTGC ATCAACTTTG
TGGCAATGAA ATTTATTGAC AATACGCTTT ACTTTATAGC
TGAAGATGAT GAAAACCTGG AATCAGATTA CTTTGGCAAG
CTTGAATCTA AATTATCAGT CATAAGAAAT TTGAATGACC
AAGTTCTCTT CATTGACCAA GGAAATCGGC CTCTATTTGA
AGATATGACT GATTCTGACT GTAGAGATAA TGCACCCCGG
ACCATATTTA TTATAAGTAT GTATAAAGAT AGCCAGCCTA
GAGGTATGGC TGTAACTATC TCTGTGAAGT GTGAGAAAAT
TTCAACTCTC TCCTGTGAGA ACAAAATTAT TTCCTTTAAG
GAAATGAATC CTCCTGATAA CATCAAGGAT ACAAAAAGTG
ACATCATATT CTTTCAGAGA AGTGTCCCAG GACATGATAA
TAAGATGCAA TTTGAATCTT CATCATACGA AGGATACTTT
CTAGCTTGTG AAAAAGAGAG AGACCTTTTT AAACTCATTT
TGAAAAAAGA GGATGAATTG GGGGATAGAT CTATAATGTT
CACTGTTCAA AACGAAGACT AG
(SEQ ID NO: 6)
ATGGCCGCCG AGCCCGTGGA GGACAACTGC ATCAACTTCG
TGGCCATGAA GTTCATCGAC AACACGCTGT ACTTCATCGC
CGAGGACGAC GAGAACCTGG AGTCCGACTA CTTCGGCAAG
CTGGAGTCCA AGTTGTCCGT CATCAGGAAC TTGAACGACC
AGGTGCTCTT CATCGACCAG GGCAACCGGC CCCTGTTCGA
GGACATGACC GACTCCGACT GCAGGGACAA CGCCCCCCGG
ACCATCTTCA TCATCAGCAT GTACAAGGAC AGCCAGCCCA
GGGGCATGGC CGTGACCATC TCCGTGAAGT GCGAGAAGAT
CTCCACCCTC TCCTGCGAGA ACAAGATCAT CTCCTTCAAG
GAGATGAACC CCCCCGACAA CATCAAGGAC ACCAAGAGCG
ACATCATCTT CTTCCAGAGG AGCGTCCCCG GCCACGACAA
CAAGATGCAG TTCGAGTCCT CCTCCTACGA GGGCTACTTC
CTGGCCTGCG AGAAGGAGAG GGACCTGTTC AAGCTCATCT
TGAAGAAGGA GGACGAGTTG GGGGACAGGT CCATCATGTT
CACCGTGCAG AACGAGGACT AG
(SEQ ID NO: 7)
ATGGCTGCTG AACCAGTAGA AGACAATTGC ATCAACTTTG
TGGCAATGAA ATTTATTGAC AATACGCTTT ACTTTATAGC
TGAAGATGAT GAAAACCTGG AATCAGATTA CTTTGGCAAG
CTTGAATCTA AATTATCAGT CATAAGAAAT TTGAATGACC
AAGTTCTCTT CATTGACCAA GGAAATCGGC CTCTATTTGA
AGATATGACT GATTCTGACT GTAGAGATAA TGCACCCCGG
ACCATATTTA TCATCAGCgc cTACggcGAC AGCagggccA
GGGGCaagGC TGTAACTATC TCTGTGAAGT GTGAGAAAAT
TTCAACTCTC TCCTGTGAGA ACAAAATTAT TTCCTTTAAG
GAAATGAATC CTCCTGATAA CATCAAGGAT ACAAAAAGTG
ACATCATATT CTTTCAGAGA AGTGTCCCAG GACATGATAA
TAAGATGCAA TTTGAATCTT CATCATACGA AGGATACTTT
CTAGCTTGTG AAAAAGAGAG AGACCTTTTT AAACTCATTT
TGAAAAAAGA GGATGAATTG GGGGATAGAT CTATAATGTT
CACTGTTCAA AACGAAGACT AG
(SEQ ID NO: 8)
ATGGCCGCCG AGCCCGTGGA GGACAACTGC ATCAACTTCG
TGGCCATGAA GTTCATCGAC AACACGCTGT ACTTCATCGC
CGAGGACGAC GAGAACCTGG AGTCCGACTA CTTCGGCAAG
CTGGAGTCCA AGTTGTCCGT CATCAGGAAC TTGAACGACC
AGGTGCTCTT CATCGACCAG GGCAACCGGC CCCTGTTCGA
GGACATGACC GACTCCGACT GCAGGGACAA CGCCCCCCGG
ACCATCTTCA TCATCAGCgc cTACggcGAC AGCagggccA
GGGGCaagGC CGTGACCATC TCCGTGAAGT GCGAGAAGAT
CTCCACCCTC TCCTGCGAGA ACAAGATCAT CTCCTTCAAG
GAGATGAACC CCCCCGACAA CATCAAGGAC ACCAAGAGCG
ACATCATCTT CTTCCAGAGG AGCGTCCCCG GCCACGACAA
CAAGATGCAG TTCGAGTCCT CCTCCTACGA GGGCTACTTC
CTGGCCTGCG AGAAGGAGAG GGACCTGTTC AAGCTCATCT
TGAAGAAGGA GGACGAGTTGGGGGACAGGT CCATCATGTT
CACCGTGCAG AACGAGGACT AG

The lower case nucleotides in SEQ ID NOs: 5-8 indicate some codons that can be altered for the variant IL-18s.

The polynucleotides may be operatively linked to one or more control sequences that control gene expression to create a recombinant polynucleotide capable of expressing the polypeptide. Expression constructs containing a heterologous polynucleotide encoding the engineered IL-18 variants can be introduced into appropriate host cells to express the corresponding IL-18 variant polypeptide.

The polynucleotide can encode an engineered IL-18 variant and can have at least 60%, 65%, 70%, 75%, 80%, 85%, 90%, 91%, 92%, 93%, 94%, 95%, 96%, 97%, 98%, 99% or more sequence identity to a reference sequence selected from: SEQ ID NO: 5-8. The polynucleotide can encode an engineered IL-18 variant and can hybridize under stringent hybridization conditions to a nucleic acid having the sequence of one of SEQ ID NO: 5-8, or a complement thereof.

The polynucleotides can be codon optimized to fit the host cell in which the protein is being produced. For example, preferred codons used in bacteria are used to express the gene in bacteria; preferred codons used in yeast are used for expression in yeast; and preferred codons used in mammals are used for expression in mammalian cells.

In another aspect, the polynucleotide encoding a IL-18 variant may be manipulated in a variety of ways to provide for expression of the polypeptide. The polynucleotides encoding the polypeptides can be provided as expression vectors where one or more control sequences are present to regulate the expression of the polynucleotides and/or polypeptides. Manipulation of the isolated polynucleotide prior to its insertion into a vector may be desirable or necessary depending on the expression vector. The techniques for modifying polynucleotides utilizing recombinant DNA methods are well known in the art. Guidance is provided in Sambrook et al., Molecular Cloning: A Laboratory Manual, 3^{rd} Ed., Cold Spring Harbor Laboratory Press (2001); and Current Protocols in Molecular Biology, Ausubel. F. ed., Greene Pub. Associates, (1998), with updates to 2006.

The control sequences can include among others, promoters, enhancers, leader sequences, polyadenylation sequences, propeptide sequences, signal peptide sequences, and transcription terminators. Other control sequences will be apparent to the person of skill in the art.

Suitable promoters can be selected based on the host cells used. For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus lichenformis alpha-amylase gene (amyL), Bacillus stearothermophilus maltogenic amylase gene (amyM), Bacillus amyloliquefaciens alpha-amylase gene (amyQ), Bacillus lichenformis penicillinase gene (penP), Bacillus subtilis xylA and xylB genes, and prokaryotic beta-lactamase gene, the tac promoter, or the T7 promoter.

Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase.

Exemplary promoters for insect cells include, among others, those based on polyhedron, PCNA, OplE2, OplE1, Drosophila metallothionein, and Drosophila actin 5 C. In some embodiments, insect cell promoters can be used with Baculoviral vectors.

Exemplary promoters for plant cells include, among others, those based on cauliflower mosaic virus (CaMV) 35S, polyubiquitin gene (PvUbi1 and PvUbi2), rice (Oryza sativa) actin 1 (OsAct1) and actin 2 (OsAct2) promoters, the maize ubiquitin 1 (ZmUbi1) promoter, and multiple rice ubiquitin (RUBQ1, RUBQ2, rubi3) promoters.

Exemplary promoters for mammalian cells include, among others, CMV IE promoter, elongation factor 1α-subunit promoter, ubiquitin C promoter, Simian Virus 40 promoter, and phosphoglycerate Kinase-1 promoter.

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used.

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region which directs the expressed polypeptide into the secretory pathway of a host cell of choice may be used in the present disclosure.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide can be converted to a mature active polypeptide by catalytic or autocatalytic cleavage of the propeptide from the propolypeptide. Where both signal peptide and propeptide regions are present at the amino terminus of a polypeptide, the propeptide region is positioned next to the amino terminus of a polypeptide and the signal peptide region is positioned next to the amino terminus of the propeptide region.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter.

In another aspect, the present disclosure is also directed to a recombinant expression vector comprising a polynucleotide encoding an IL-18 variant, and one or more expression regulating regions such as a promoter and a terminator, a replication origin, etc., depending on the type of hosts into which they are to be introduced.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The expression vector can exist as a single copy in the host cell, or maintained at higher copy numbers, e.g., up to 4 for low copy number and 50 or more for high copy number.

In some embodiments, the expression vector contains one or more selectable markers, which permit selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus lichenformis, or markers, which confer antibiotic resistance such as ampicillin, kanamycin, chloramphenicol (Example 1) or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

The expression vector may be a bi-cistronic construct or multiple cistronic construct. The two cistrons may be oriented in opposite directions with the control regions for the cistrons located in between the two cistrons. When the construct has more than two cistrons, the cistrons may be arranged in two groups with the two groups oriented in opposite directions for transcription. Exemplary bicistronic constructs are described in Amendola et al., Nat. Biotechnol. 23:108-116 (2005), which is incorporated by reference in its entirety for all purposes. The control region for one cistron may be capable of high transcription activity and the other may have low transcriptional activity under conditions of use. One or both control regions may be inducible. Examples of high transcription activity control regions include, for example, MND, EF1-alpha, PGK1, CMV, ubiquitin C, SV40 early promoter, tetracycline-responsive element promoter, cell-specific promoters, human beat-actin promoter, and CBG (chicken beta-globin), optionally including the CMV early enhancer. Examples of low transcription activity control regions include, for example, TRE3G (commercially sold by Clontech, a tetracycline-responsive element promoter with mutations that reduce basal expression), T-REx™ (commercially sold by ThermoFisher), and a minimal TATA promoter (Kiran et al., Plant Physiol. 142:364-376 (2006), which is incorporated by reference in its entirety for all purposes), HSP68, and a minimal CMV promoter. Examples of inducible control regions include, for example, NFAT control regions (Macian et al, Oncogene 20:2476-2489 (2001)), and the inducible control regions described above.

The bi-cistronic construct may encode a CAR and a polypeptide that is a payload (or makes a payload) to be delivered at a target site. Exemplary payloads are described above and below. The nucleic acid encoding the CAR can be operably linked to a strong promoter, a weak promoter, and/or an inducible promoter, and optionally, operably linked to a RNA control device, DE, RDE, or combination of the foregoing. The CAR can be encoded by nucleic acids in a Side-CAR format. The nucleic acid encoding the polypeptide can be operably linked to a strong promoter, a weak promoter, and/or an inducible promoter. The nucleic acid encoding the polypeptide that is a payload (or makes the payload) can be under the control of an RDE. The RDE may be one that responds to the activation state of the cell through, for example, glycolytic enzymes such as, for example, glyceraldehyde phosphate dehydrogenase (GAPDH), enolase (ENO1 or ENO3), phosphoglycerate kinase (PGK1), triose phosphate isomerase (TPI1), aldolase A (ALDOA), or phosphoglycerate mutase (PGAM1). The RDE may also be bound and regulated by other energy metabolism enzymes such as, for example, transketolase (TKT), malate dehydrogenase (MDH2), succinyl CoA Synthetase (SUGLG1), ATP citrate lyase (ACLY), or isocitrate dehydrogenase (IDH1/2). The host cell can express a CAR that binds to its antigen at a target site in a subject. This binding of antigen at the target site activates the cell causing the cell to increase glycolysis which induces expression of the nucleic acid encoding the polypeptide under the control of the RDE (bound by glycolytic or other energy metabolism enzymes).

The multicistronic constructs can have three or more cistrons with each having control regions (optionally inducible) and RDEs operably linked to some or all of the transgenes. These cassettes may be organized into two groups that are transcribed in opposite directions on the construct. Two or more transgenes can be transcribed from the same control region and the two or more transgenes may have IRES (internal ribosome entry site) sequences operably linked to the downstream transgenes. Alternatively, the two or more transgenes are operably linked together by 2A elements as described in Plasmids 101: Multicistronic Vectors found at blog.addgene.org/plasmids-101-multicistrnic-vectors. Commonly used 2A sequences include, for example, EGRGSLLTCGDVEENPGP (T2A) (SEQ ID NO: 9), ATNFSLLKQAGDVEENPGP (P2A) (SEQ ID NO: 10); QCTNYALLKLAGDVESNPGP (E2A) (SEQ ID NO: 11); and VKQTLNFDLLKLAGDVESNPGP (F2A) (SEQ ID NO: 12) all of which can optionally include the sequence GSG at the amino terminal end. This allows multiple transgenes to be transcribed onto a single transcript that is regulated by a 3′-UTR with an RDE (or multiple RDEs).

The bicistronic/multicistronic vector can increase the overall expression of the two or more cistrons (versus introducing the cistrons on separate constructs). The bicistronic/multicistronic construct can be derived from a lenti-virus vector. The bicistronic/multicistronic construct can encode a CAR and a polypeptide(s) that is encoded on a transgene(s) (e.g., a payload), and the bicistronic construct may increase expression of the polypeptide encoded by the transgene(s) when the cell is activated by the CAR.

### RNA Destabilizing Elements

RNA destabilizing elements (RDE) are nucleic acids that affect or maintain the stability of an RNA molecule or the translation kinetics of an RNA molecule. Some RDEs are bound by polypeptides which destabilize (e.g., cleave) the RNA, or prevent translation, leading to loss of function for the RNA. Some RDE binding polypeptide stabilizes the RNA increasing the half-life of the RNA. RDEs can be used to control the expression of a transgene, e.g., a transgene encoding a chimeric antigen receptors. RDEs can be used with RNA control devices, DEs, and/or Side CARs to regulate the expression of a transgene. The RDEs can also be used to control expression of transgenes encoding polypeptides other than a CAR. Other transgenes may encode, for example, a cytokine, an antibody, a checkpoint inhibitor, a granzyme, an apoptosis inducer, complement, a cytotoxic small molecule, other cytotoxic compounds, a polypeptide for imaging, or other polypeptide that can have a desired effect. The RDE can control the delivery of a transgene payload. Examples of RDEs include, for example, AU rich elements, U rich elements, GU rich elements, and certain stem-loop elements. Exemplary RDEs are described in Kovarik et al., Cytokine 89:21-26 (2017); Ray et al., Nature 499:172-177 (2013); Castello et al., Cell 149:1393-1406 (2012); Vlasova et al., Mole. Cell. 29:263-270 (2008); Barreau et al., Nucl. Acids Res. vol 33, doi:10.1093/nar/gkil012 (2006); Meisner et al., ChemBioChem 5:1432-1447 (2004); Guhaniyogi et al., Gene 265:11-23 (2001), all of which are incorporated by reference in their entirety for all purposes.

The RDE can be a Class I AU rich element (dispersed AUUUA (SEQ ID NO:13) in U rich context), a Class II AU rich element (overlapping (AUUUA)ₙ), a Class III AU rich element (U-rich stretch), a stem-loop destabilizing element (SLDE), a cytokine 3′ UTR (e.g., INF-γ, IL-2, T-cell receptor a chain, TNFα, IL-6, IL-8, GM-CSF, G-CSF etc.), and a sequence of AUUUAUUUAUUUA (SEQ ID NO: 14). Khabar, WIREs RNA 2016, doi: 10.1002/wrna.1368 (2016); Palanisamy et al, J. Dent. Res. 91:651-658 (2012), both of which are incorporated by reference in their entirety for all purposes. The RDE can also be a GU rich element comprised of one or more of, for example, UUGUU (SEQ ID NO: 15), UGGGGAU (SEQ ID NO: 16), or GUUUG (SEQ ID NO: 17). The RDE can be a U-rich element comprised of one or more of, for example, UUUGUUU (SEQ ID NO: 18), NNUUNNUUU (SEQ ID NO: 19), UUUAUUU (SEQ ID NO: 20), UUUUUUU (SEQ ID NO: 21), UUAGA (SEQ ID NO: 22), or AGUUU (SEQ ID NO: 23). In some aspects, multiple RDEs can be combined to make a regulatory unit, for example, multiple RDEs that have the same sequence can be arranged in a concatemer or can be arranged with intervening sequence in between some or all of the RDEs. The RDE sequence can be modified to increase or decrease the affinity of an RNA binding protein(s) for the RDE. For example, an AU rich RDE can be changed to alter the affinity of glyceraldehyde phosphate dehydrogenase (GAPDH) to the RDE. This change in affinity can alter the GAPDH-activation threshold for expression of a transgene regulated by the RDE to which GAPDH binds.

The disclosure assigns AU #designations to some RDEs and these RDEs can be referred to by the AU # or the gene name from which the RDE is derived. Some AU #s and the corresponding gene from which the RDE is derived include, for example, AU 1 (CD40LG), AU 2 (CSF2), AU 3 (CD247), AU 4 (CTLA4), AU 5 (EDN1), AU 6 (IL2RA), AU 7 (SLC2A 1), AU 8 (TRAC), AU 9 (CD274), AU 10 (Myc), AU 11 (CD19), AU 12 (IL4), AU 13 (IL5), AU 14 (IL6), AU 15 (IL9), AU 16 (IL10), AU 17 (IL13), AU 18 (FOXP3), AU 19 (TMEM-219), AU 20 (TMEM-219snp), AU 21 (CCR7), AU 22 (SEM-A4D), AU 23 (CDC42-SE2), AU 24 (CD8), AU 27 (bGH), and AU 101 (IFNg).

The RDE can be from the 3′ UTR of a gene encoding, for example, IL-1, IL-2, IL-3, IL-4, IL-6, IL-8, IL-10, GM-CSF, G-CSF, VEG F, PGE₂, COX-2, MMP (matrix metalloproteinases), bFGF, c-myc, c-fos, beta1-AR, PTH, interferon-gamma, MyoD, p21, Cyclin A, Cyclin B1, Cyclin D1, PAI-2, NOS HANOS, TNF-alpha, interferon-alpha, bcl-2, interferon-beta, c-jun, GLUT1, p53, Myogenin, NF-M, or GAP-43, lymphocyte antigen 96, SUPV3L1, SFtPA2, BLOC1S2, OR10A6, OR8D1, TRPT1,CIP29, EP400, PLE2, H3ST3A1, ZNF571, PPP1R14A, SPAG4L, OR10A6 and KIR3DL. Other RDEs are found in, for example, the 3′-UTRs from GLMN, AMY2B, AMY2A, AMY2A, AMYlA, TRIM33, TRIM33, TRIM33, CSRP1, PPP1R12B, KCNH1, Reticulon_4, MRPL30, Nav1.2, Tissue_factor_pathway_inhibitor, EEF1B2, CRYGB, ARMC9, RPL15, EAF2, MRPS22, MRPS22, COPB2, PDCD10, REl-silencing transcription_factor, Amphiregulin, AP1AR, TLR3, SKP2, Peptidylglycine_alpha-amidating_monooxygenase, TNFAIP8, Interleukin_9, PCDHA2, PCDHA12, Aldehyde_dehydrogenase_5_family, _member_A1, KCNQ5, COX7A2, Monocarboxylate transporter 10, MLLT4, PHF10, PTPN12, MRNA_(guanine-N7-)-methyltransferase, WHSC1L1, Tricho-rhino-phalangeal_syndrome_Type_1, Interferon alpha-1, ZCCHC6, Retinitis_pigmentosa_GTPase_regulator, MED14, CLCN5, DNA2L, OR52D1, NELL1, SLC22A25, SLC22A10, TRPC6, CACNA2D4, EPS8, CT2_(gene), Mitochondrial_ribosomal_protein_L42, TAOK3, NUPL1, Endothelin_receptor_type_B, Survival_of_motor_neuron_protein-interacting_protein_1, POLE2, Hepatic_lipase, TPSG1, TRAP1, RPS15A, HS3ST3A1, CROP_(gene), Apolipoprotein_H, GRB2, CEP76, VPS4B, Interleukin_28B, IZUMO1, FGF21, PPP1R15A, LIN7B, hnRNPLL, Tox, and CDC45-related_protein.

Still other RDEs can be found in, for example, the 3′UTRs of SCFD1, MAL2, KHSRP, IQCB1, CAMP_responsive_element_modulator, MFAP5, SBF2, FKBP2, PDCD10, UBE2V2, NDUFAB1, Coiled-Coil_Domain_Containing_Protein, ALG13, TPTE, Enaptin, Thymopoietin, Delta-like_i, C11orf30, Actinin_alpha_4, TMEM59, SP 110, Dicer, TARDBP, IFNA17, IFNA16, IFNA14, ZMYM3, Interleukin_9, type_I, OPNISW, THSD1, ERGIC2, CAMK2B, WDR8, FXR1, Thymine-DNA_glycosylase, Parathyroid_hormone-related_protein, OSBPL3, Ran, GYPE, AKAP4, LOC642658, L2HGDH, AKAP1, Zinc_finger_protein_334, TC2N, FKBPL, GRB14, CXorf67, CXorf66, CEP76, Gastricsin, CEP70, CYP26A1, NAA35, Aryl_hydrocarbon_receptor_nuclear_translocator, KLC4, GPR112, LARP4, NOVA1, UBE2D3, ITGA6, GPR18, MGST type_A, REi-silencing transcription_factor, ASPM, ZNF452, KIR2DS4, AHSA1, TMTC4, VSX1, P16, MRPL19, CCL20, TRPT1, Hepatic lipase, PDLIM5, CCDC53, ′CCDC55, GAPVD1, HOXB2, KCNQ5, BRCC3, GTF2IRD1, CDK5RAP3, Transcription_factor_II_B, ZEB1, IRGM, SLC39A6, RHEB, PSIP1, RPS6KA5, Urokinase_receptor, GFM1, DNAJC7, Phosphoinositide-dependent_kinase-1, LMOD3, TTC35, RRP12, ATXN2, ACSM3, SOAT1, FGF8, HNRPH3, CTAGE5, POLG2, DYRK3, POLK, Cyclin-dependent_kinase_inhibitor_1C, CD137, Calmodulin_i, ZNF571, CNOT2, CRYZLI, SMC3, SMC4, SLC36A1, Decorin, HKRI, ERC1, S100A6, RIMS1, TMEM67, Mitochondrial_ribosomal_protein_L42, MECP2, RNF111, SULTIA1, MYLK3, TINAG, PRKAR1A, RGPD5, UBE2V1, SAR1B, SLC27A6, ZNF638, RAB33A, TRIOBP, MUCL1, CADPS2, MCF2L, TBCA, SLC17A3, LEO1, IFNA21, RUNX1T1, PRKD2, ATP11B, MORC2, RBM6, KLRD1, MED31, PPHLNI, HMGB2, DNA_repair_and_recombination_protein_RAD54-like, RBM9′, ARL11, HuD, SPEF2, CBLL1, SLC38A1, ‘Caspase_1’, S100G, CAi_, CELAi, PTS, ITM2B, Natriuretic_peptide_precursor_C, TRPP3, IMPDH2, DPYS, CDCA3, EFCAB6, SLIT2, SIPAILI, FIP1L1, ATP6V1B2, HSD17B4, HSD17B7, NDUFCI, CROP, CD48, APPBPI, CD44, CD46, Histone_deacetylase_2_type_XI, Interleukin_4, Tricho-rhino-phalangeal_syndrome_Type_1, SEC61G, TRIP12, PLEKHO1, SEC61B, ST6GALNACI, CPVL, E2F7, UTP20, E2F5, PARD3, EXOC7, HEXB, Caspase_recruitment_domain-containing_protein_8, MBD4, PPP4C, Helicase, Phosducin, SPG11, CGGBP1, PSKH1, Cathepsin_S, orexin, IMMP2L, C2orf28, Laminin, EIF3S6, LRRC41 type XII, Cathepsin_C, HPS6, ARAF, Zinc_finger_and_BTB_domain-containing_protein_16, Sex_hormone-bindingglobulin, FBLN2, Suppressor_of_cytokine_signaling_1, TMEM126A, DOM3Z, TSFM_POLQ-like, DYNLT3, CDH9, EAF2, MIPEP, NDUFA12, HDAC8, MKKS, FGG, IL36G, CDCA7, CRISPLD2, Olfactomedin-like_2b, MRPL32, MRPL33, AHIl, SMARCAL1, UTP14A, SSH2, Dystonin, Contactin_6, PPFIBP1, THOC1, CNOT1, RHCE, SLC41A3, SLC2A9, SNAP23, RFX3, GNG4, MRPL40, LSR, Angiogenin, TRIP4, VRK1, COUP-TFII, FOXP2, SNX2, Nucleoporin_85, RPL37A, RPL27A, SEC62, Calcium-activated_potassium_channel_subunit_alpha-1, SMARCE1, RPL17, CEP104, CEP290, VPS29, ANXA4, Zinc_finger_protein_737, DDX59, SAP30, NEK3, Exosome_component_9, Receptor_for_activated_C_kinase_1, Peptidylprolyl_isomerase_A, TINP1, CEACAMI, DISCI, LRRTM1, POP1_Lamin_B1, SREBP_cleavage-activating_protein, COX6C, TLR_1, ARID2, LACTB, MMS22L, UBE2E3, DAP3, ZNF23, SKP2, GPR113, IRF9 Ghrelin_O-acyltransferase, NEIL3, EEF1E1, COX17, ESD_, Dentin_sialophosphoprotein, HDAC9, RFC4, CYLD, RPLPO, EIF2B3, UGT2A1, FABP7, TRIP11, PLA2G4A, AKR1C3, INTS12, MYH1, ZBTB17, MYH4, NLRP2, MECOM, MYH8, Thermogenin_receptor_2, IFI16, THYN1, RAB17, ETFA, Cystic_fibrosis_transmembrane_conductance_regulator, F13B, RAB6A, ST8SIA1, SATB2, SATB1, HMG20B, UHRF1, CNOT3, Prostaglandin_EP2_receptor, FAM65B, Peroxisome_proliferator-activated_receptor_gamma, KvLQT2, GRIK5, SHOC2, Cortactin, FANCI, KIAA1199, Kynureninase, Decoy_receptor_1, NEU3, PHF10, Methyl-CpG-binding_domain_protein_2, RABGAP1, CEP55, SF3B1, MSH5, MSH6, CREB-binding_protein, LIMS1, SLC5A4, CCNB1IP1, RNF34, SORBS2, UIMC1, SOX5, YWHAZ, ICOSLG, NOP58, Zinc finger_protein_679, PHKB, MED13, ABCB7, COQ9, C14orf104, Zinc_finger_protein_530, KLRC2, LSM8, NBR1, PRKCD, Long-chain-aldehyde_dehydrogenase, MTSS1, Somatostatin, Ubiquitin_carboxyl-terminal hydrolase_L5, WDR72, FERMT3, Nuclear receptor_related-lprotein, Citrate_synthase, VPS11, KIZ, ZFYVE27, BCKDHB, Hypocretin, CACNG2, PTCH1, Carbonic_anhydrase_4, Nucleoporin_107, LDL_receptor, LEKTI, FBXO11, NDUFB3, FCHO2, CEP78, RAPGEF6, PPIL3, NIN, RAPGEF2, Growth_hormone 1, Growth_hormone_2, MNAT1, Nav1, MAP3K8, SUGT1, LAIR1, Hyaluronan-mediated motility receptor, MAP3K2, MPP2, TFB2M, CRB3, MPP5, CACNAlG, DLGAP2, INHBA, MAGI2, CIP29, SETDB1, Cytochrome_b5, TRPV2, Interleukin_1_receptor, HOXD8, TIMM10, ATXN2L, CLCN2, CREB1, TNIP1, CBLB, Factor_V, USP33, SON, RBBP8, SLC22A18, PTPN12, ADCY8, MYLK, KIF23, REXO2, BST1, TOP3B, COPB1, AXIN2, COPB2, TNRC6B, Guanidinoacetate_N-methyltransferase, Acyl-CoA_thioesterase_9, C4orf21, TSHB, FRS3, EPB41, Cyclin_T2, LAIR2, Nucleoporin_43, APLP2, TNFRSF19, Death-associated_protein_6, Epithelial_cell_adhesion_molecule, CLEC7A, Gephyrin, CLDND1, VPS37A, PCDHAC2, Bone_morphogenetic_protein_4, NVL, RBM33, RNF139, Sperm_associated_antigen_5, PLCB1, Glial_cell_line-derived_neurotrophic_factor, PARP4, PARP1, MAN2A1, Bone_morphogenetic_protein_1, PAX4, BCCIP, MMP7, Decoy_receptor_3, RAMP2, NCAPD3, LRRC37A, RWDD3, UBE2A, UBE2C, SLC3A1, MRPS22, CDCl4A, ITSN1, POLE2, MYC-induced_nuclear_antigen, TMLHE, Glutamate_carboxypeptidase_II, GPR177, PPP2R5C, KIAA1333, RPP38, MYO1F, Farnesoid X_receptor, Caldesmon, FBXO4, FBX05, OPN1MW, PIGN, ARNTL2, BCAS3, C6orf58, PHTF2, SEC23A, NUFIP2, OAZ1, Osteoprotegerin, ANAPC4, ATP6VOA2, SPAM1, PSMA6, TAS2R30, RABEPI, DPM3, SLC6A15, RPS26, RPS27, RPS24, RPS20, RPS21, ARHGAP24, Catechol-O-methyl transferase, ERCC5, Transcription_initiation_protein_SPT3_homolog, OR1E1, ZNRF1, GMEB1, CCT2_GNAQ, Mucin_6, Mucin_4, LRP5, PDE9A, C2orf3, EZH2, Epidermal_growth_factor_receptor, TMTC2, PDE4A, EPH_receptor_A4, PPIB, DENND4A, ANTXR1, ANTXR2, Nucleoporin_88, SLCO1B3, COG8, RBMS1, MAP7, HIST2H2BE, AEBP2, DCLRElA, RPL24, HNRPA2B1, RPL21, RPL23, MAPKAP1, NIPBL, ATG7, SERPINI2, GYLTL1B, ATP5G2, DIP2A, AMY2A, CEP63, TDRD7, PIEZO1, CLDN20, GRXCR1, PMEL, NIF3L1, MCC_, PCNX, TMBIM4, DUSP12, ZMYND8, GOSRI, Interferon_gamma_receptor_1, LDB3, PON3, CID, ABCC8, COQ7, COQ6, AMELY, HAVCR1, PICALM, Sjogren_syndrome_antigen_B, PLK4, HBB, AKT1, PCDHGB7, C6orf10, UBR1, Retinoblastoma-like_protein_1, GRK6, WWC2, GRK4, INPP4B, SLC34A1, GOLGA2, MYCBP2, PTP4A2, NUCB2, MAGOH, RPP40, Alpha-2A_adrenergic_receptor, SPAG11B, Nucleoporin_205, COG1, Motile_sperm_domain_containing_3, KCNMB3, Motile_sperm_domain containing_1, KLHL7, KCNN2, TSPAN8, GPR21, Translocator_protein, HNRNPLL, ABHD5, CAB39L, Amphiregulin, GPR1, Interleukin_18, EIF4G3, Interleukin_15, CCDC80, CD2AP, NFS1, GRB2, ULBP2, Vascular_endothelial_growth_factor_C, RPS3, TLR8, BCL2-related_protein_A1, RHOT1, Collagen, Centromere_protein_E, STMN2, HESX1, RPL7, Kalirin, PCMT1, HLA-F, SUMO2, NOX3, EP400, DNM3, EED, NGLY1, NPRL2, PLAC1, Baculoviral IAP_repeat-containing_protein_3, C7orf31, TUBAIC, HAUS3, IFNA10, MYST4, DCHS1, SIRT4, EFEMP1, ARPC2, MED30, IFT74, PAKIIPI, DYNC1LI2, POLR2B, POLR2H, KIF3A, PRDM16, PLSCR5, PEX5, Parathyroid_hormone_1_receptor, CDC23, RBPMS, MAST1, NRD1, BAT5, BAT2, Dock 11, GCSH, POF1B, USP15, POT1, MUTYH, CYP2E1, FAM122C, Alpolypeptide, Flavin_containing_monooxygenase_3, HPGD, LGALS13, MTHFD2L, Survival_motor_neuron_domain_containing_1, PSMA3, MRPS35, MHC_class_I_polypeptide-related_sequence_A, SGCE, REPS1, PPP1R12A, PPP1R12B, PABPC1, MAPK8, PDCD5, Phosphoglucomutase_3, Ubiquitin_C, GABPB2, Mitochondrial_translational_release_factor_1, PFDN4, NUB1, SLC13A3, ZFP36L1, Galectin-3, CC2D2A, GCA, Tissue_factor_pathway_inhibitor, UCKL1, ITFG3, SOS1, WWTR1, GPR84, HSPA14, GJC3, TCF7L1, Matrix_metallopeptidase_12, ISG20, LILRA3, Serum_albumin, Phosducin-like, RPS13, UTP6, HP1BP3, IL12A, HtrA_serine_peptidase_2, LATS1, BMF_, Thymosin_beta-4, B-cell_linker, BCL2L11, Coagulation_factor XIII, BCL2L12, PRPF19, SFRS5, Interleukin_23_subunit_alpha, NRAP, 60S_ribosomal_protein_L14, C9orf64, Testin, VPS13A, DGKD, PTPRB, ATP5C1, KCNJ16, KARS, GTF2H2, AMBN, USP13, ADAMTSL1, TRO_, RTF1, ATP6V1C2, SSBP1, SNRPN_upstream_reading_frame_protein, RPS29, SNRPG, ABCC10, PTPRU, APPL1, TINF2, TMEM22, UNC45A, RPL30, PCDH7, Galactosamine-6_sulfatase, UPF3A, ACTL6A, ACTL6B, IL3RA, SDHB, Cathepsin_L2, TAS2R7, Cathepsin_L1, Pituitary_adenylate_cyclase-activating_peptide, RPN2, DYNLL1, KLK13, NDUFB3, PRPF8, SPINT2, AHSA1, Glutamate_carboxypeptidase_II, DRAP1, RNASE1, Olfactomedin-like_2b, VRK1, IKK2, ERGIC2, TAS2R16, CAMK2G, CAMK2B, Estrogen_receptor_beta, NADH_dehydrogenase, RPL19, NUCB2, KCTD13, ubiquinone, H2AFY, CEP290, PABPC1, HLA-F, DHX38, KIAA0922, MPHOSPH8, DDX59, MIB2_, ZBP1, C16orf84, UACA, C6orf142, MRPL39, Cyclin-dependent_kinase_7, Far upstream_element-binding_protein_1, SGOL1, GTF2IRD1, ATG10, Dermcidin, EPS8L2, Decorin, Nicotinamide_phosphoribosyltransferase, CDC20, MYB, WNT5A, RBPJ, DEFB103A, RPS15A, ATP5H, RPS3, FABP1, SLC4A8, Serum_amyloid_P_component, ALAS1, MAPK1, PDCD5, SULTIA1, CHRNA3, ATXN10, MNAT1, ALG13, Ataxin 3, LRRC39, ADH7, Delta-sarcoglycan, TACC1, IFNA4, Thymic_stromal_lymphopoietin, LGTN, KIAA1333, MSH6, MYOT, RIPK5, BCL2L11, RPL27, Rndl, Platelet_factor_4, HSD17B7, LSM8, CEP63, INTS8, CTNS, ASAHL, CELA3A, SMARCAL1, HEXB, SLC16A5, MAP3K12, FRMD6.

Additional RDEs are found in the 3′-UTRs of long noncoding RNAs, or primary transcripts encoding miRNAs. For example, RDEs from the 3′-UTR of THRIL (line 1992), NIKILA lncRNA, SeT lncRNA, lncRNAs uc.197, RP11-172E9.2, LINC00598, lncRNAs LOC100128098, RP11-150012.3, and the primary transcripts encoding miR-146a, miR-let7e, miR-181c, miR-155, miR-125b, and miR-16.

A class of RDEs includes those which are bound by glycolytic enzymes such as glyceraldehyde phosphate dehydrogenase (GAPDH). This group of RDEs includes, for example, AU 19 (TMEM-219), AU 20 (TMEM-219snp), AU 21 (CCR7), AU 22 (SEM-A4D), and AU 23 (CDC42-SE2).

The RDE can be a Class I AU rich element that arises from the 3′ UTR of a gene encoding, for example, c-myc, c-fos, beta1-AR, PTH, interferon-gamma, MyoD, p21, Cyclin A, Cyclin B1, Cyclin D1, PAI-2, or NOS HANOS. The RDE can also be a Class II AU rich element and arises from the 3′ UTR of a gene encoding, for example, GM-CSF, TNF-alpha, interferon-alpha, COX-2, IL-2, IL-3, bcl-2, interferon-beta, or VEG-F. The RDE can be a Class III AU rich element that arises from the 3′ UTR of a gene encoding, for example, c-jun, GLUT1, p53, hsp 70, Myogenin, NF-M, or GAP-43. Other RDEs may be obtained from the 3′-UTRs of a T-cell receptor subunit (α, β, γ, or δ chains), cytotoxic T-lymphocyte-associated antigen 4 (CTLA4), programmed cell death protein (PD-1), Killer-cell Immunoglobulin-like Receptors (KIR), and Lymphocyte Activation Gene-3 (LAG3), and other checkpoint inhibitors. Still other RDEs may be obtained from the 3′-LUTRs ofsenescence-associated secretory phenotypc genes disclosed in Coppe et al, Ann. Rev, Pathol. 5:99-118 (2010), which is incorporated by reference in its entirety for all purposes (e.g, see Table 1).

The RDE can be bound by certain polypeptides including, for example, ARE poly(U) binding/degradation factor (AUF-1), tristetraprolin (TTP), human antigen-related protein (HuR), butyrate response factor 1 (BRF-1), butyrate response factor 2 (BRF-2), T-cell restricted intracellular antigen-1 (TIA-1), TIA-1 related protein (TIAR), CUG triplet repeat, RNA binding protein 1 (CUGBP-1), CUG triplet repeat, RNA binding protein 2 (CUGBP-2), human neuron specific RNA binding protein (Hel-N1, Hel-N2), RNA binding proteins HuA, HuB and HuC, KH-type splicing regulatory protein (KSRP), 3-methylglutaconyl-CoA hydratase (AUH), glyceraldehyde 3-phosphate dehydrogenase (GAPDH), heat shock protein 70 (Hsp70), heat shock protein 10 (Hsp10), heterogeneous nuclear ribonucleoprotein A1 (hnRNP A1), heterogeneous nuclear ribonucleoprotein A2 (hnRNP A2), heterogeneous nuclear ribonucleoprotein A3 (hnRNP A3), heterogeneous nuclear ribonucleoprotein C (hnRNP C), heterogeneous nuclear ribonucleoprotein L (hnRNP L), Bcl-2 AU-rich element RNA binding protein (TINO), Poly(A) Binding Protein Interacting Protein 2 (PAIP2), IRP1, pyruvate kinase, lactate dehydrogenase, enolase, and aldolase. The RDE binding protein also can be an enzyme involved in glycolysis or carbohydrate metabolism, such as, for example, Glyceraldehyde Phosphate Dehydrogenase (GAPDH), enolase (ENO1 or ENO3), Phosphoglycerate Kinase (PGK1), Triosephosphate Isomerase (TPI1), Aldolase A (ALDOA), Phosphoglycerate Mutase (PGAM1), Hexokinase (HK-2), or Lactate Dehydrogenase (LDH). The RDE binding protein can be an enzyme involved in the Pentose Phosphate Shunt, including for example, Transketolase (TKT) or Triosephosphate Isomerase (TPI1). Additional exemplary RNA binding proteins are those described in Castello et al., Molc. Cell 63:696-710 (2016); Kovarik et al., Cytokine 89:21-26 (2017); Ray et al., Nature 499:172-177 (2013); Castello et al., Cell 149:1393-1406 (2012); Vlasova et al., Molc. Cell. 29:263-270 (2008); Barreau et al., Nucl. Acids Res. vol 33, doi:10.1093/nar/gkil012 (2006); Meisner et al., ChemBioChem 5:1432-1447 (2004); Guhaniyogi et al., Gene 265:11-23 (2001), all of which are incorporated by reference in their entirety for all purposes.

The RDE binding protein can be TTP which can bind to RDEs including for example, one or more of UUAUUUAUU (SEQ ID NO: 24) and AUUUA (SEQ ID NO: 13), or KSRP which binds AU-rich RDEs, or Aufl which binds RDEs including for example, one or more of UUGA (SEQ ID NO: 13), AGUUU (SEQ ID NO: 23), or GUUUG (SEQ ID NO: 17), or CELF-1 which binds RDEs including for example, one or more of UUGUU (SEQ ID NO: 15), or HuR which binds RDEs including for example, one or more of UUUAUUU (SEQ ID NO: 20), UUUUUUU (SEQ ID NO: 21), or UUUGUUU (SEQ ID NO: 18), or ESRP1 or ESRP2 which binds RDEs including for example, one or more of UGGGGAU (SEQ ID NO: 25), or ELAV which binds RDEs including for example, one or more of UUUGUUU (SEQ ID NO: 18). The RDE binding protein can be an enzyme involved in glycolysis, including for example, GAPDH which binds AU rich elements including for example, one or more of AUUUA (SEQ ID NO: 13) elements, or ENO3/ENO1 which binds RDEs including for example, one or more of CUGCUGCUG (SEQ ID NO: 26), or ALDOA which binds RDEs including for example, one or more of AUUGA (SEQ ID NO: 27).

In an aspect, the RDE can be combined with an RNA control device to make the regulation by the RDE ligand inducible. For example, an RDE can be operably linked to an RNA control device where ligand binding by the RNA control device activates the regulatory element (e.g., a ribozyme or riboswitch) which inhibits the RDE (e.g., a ribozyme cleaves the RDE so RDE binding proteins no longer bind, or the riboswitch alters secondary structure). This places transcripts with the RDE and RNA control device under two types of control from the RDE, first the RDE can regulate the transcript subject to binding of RDE binding proteins as governed by conditions in the cell, and second, the RDE control can be removed by inducing the RNA control device with ligand. When ligand is added, the RNA control device renders the RDE unavailable for binding and RDE regulation is removed. When ligand is removed, new transcripts that are transcribed can be under the control of the RDE (as the RNA control device will not be activated). Alternatively, an RDE can be operable linked to an RNA control device where ligand binding turns off the regulatory element (e.g., a ribozyme). In this example, the presence of ligand inhibits the RNA control device and transcripts can be regulated by the RDE. When ligand is removed, the RNA control device renders the RDE unavailable for binding to RDE binding proteins and RDE regulation of the transcript is removed. The RNA control device could also cleave a polynucleotide that binds to the RDE to form a structure (e.g., a helix) that inhibits RDE proteins from binding to the RDE. In this example, the RNA control device can cleave the inhibitory polynucleotide which then does not bind or is inhibited for binding to the RDE. This cleavage by the RNA control device can be stimulated by ligand binding or inhibited by ligand binding.

Different RDEs have different kinetic parameters such as, for example, different steady expression levels, different Tₘₐₓ (time to maximal expression level), different Cₘₐₓ (maximum expression level), different dynamic range (expression/basal expression), different AUC, different kinetics of induction (acceleration of expression rate and velocity of expression rate), amount of expression, baseline expression, maximal dynamic range (DRₘₐₓ), time to DRₘₐₓ, area under the curve (AUC), etc. In addition, these kinetic properties of the RDEs can be altered by making concatemers of the same RDE, or combining different RDEs into regulatory units. Placing RDEs under the control of an operably linked RNA control device can also alter the kinetic properties of the RDE, RDE concatemer, or RDE combinations. Also, small molecules and other molecules that affect the availability of RDE binding proteins for binding RDEs can be used to alter the kinetic response of an RDE, RDE concatemer, and/or RDE combinations. The kinetic response of RDEs, RDE concatemers, and/or RDE combinations can be changed using constructs that express competitive RDEs in a transcript. Such transcripts with one or more competing RDEs can compete for RDE binding proteins and so alter the regulation of the desired gene by an RDE, RDE concatemer, and/or RDE combination. These competitive RDE transcripts can bind to RDE binding proteins reducing the amount of RDE binding protein available for binding to the RDE, RDE concatemer, and/or RDE combination. Thus, RDEs, RDE concatemers, and/or RDE combinations can be selected and/or combined with other conditions (discussed above) to provide a desired kinetic response to the expression of a transgene.

Table 2 in Example 20 shows that different RDEs (e.g., AU elements) provided different kinetics of expression. For example, different RDEs (e.g., AU elements) reached maximal induction (maximal dynamic range also known as fold induction) at different time points. The RDEs AU 2 and AU 101 reached maximal dynamic range (DRₘₐₓ) at day 1 and then the dynamic range (DR) decreased showing reduced expression compared to basal expression. The RDEs AU 5 and AU 21 had a DRₘₐₓ at day 3/4 and this expression was maintained out to day 8. The RDEs AU 3, AU 7, AU 10, AU 20 and AU 23 had a DRₘₐₓ on day 6 and expression decreased on day 8. The RDEs AU 19 and AU 22 had DRₘₐₓ on day 8. The RDEs (e.g., AU elements) also had differences in the amount of expression covering a range of about 5500 fold comparing the expression of AU 7 to AU 10 (see Table 1). Thus, RDEs (AU elements) can be selected to provide maximal rates of expression at a desired time point and to provide a desired amount of polypeptide at that time point.

Some RNA binding proteins increase the rate of RNA degradation after binding to the RDE. Some RNA binding proteins decrease the rate of degradation of the RNA after binding to the RDE. More than one RNA binding protein binds can bind to an RDE. In some RDE regulatory units, more than one RNA binding protein binds to more than one RDE. Binding of one or more of the RNA binding proteins to the one or more RDEs can increase the degradation rate of the RNA. Binding of one or more of the RNA binding proteins can decrease the degradation rate of the RNA. RNA binding proteins that increase degradation may compete for binding to an RDE with RNA binding proteins that decrease degradation, so that the stability of the RNA is dependent of the relative binding of the two RNA binding proteins. Other proteins can bind to the RDE binding proteins and modulate the effect of the RNA binding protein on the RNA with the RDE. Binding of a protein to the RNA binding protein can increases RNA stability or decrease RNA stability. An RNA can have multiple RDEs that are bound by the proteins HuR and TTP. The HuR protein can stabilize the RNA and the TTP protein can destabilize the RNA. An RNA can have at least one RDE that interacts with the proteins KSRP, TTP and/or HuR. KSRP can destabilize the RNA and compete for binding with the HuR protein that can stabilize the RNA. The KSRP protein can bind to the RDE and destabilizes the RNA and the TTP protein can bind to KSRP and prevent degradation of the RNA. Different proteins may be bound to the same transcript and may have competing effects on degradation and stabilization rates. Different proteins may be bound to the same transcript and may have cooperative effects on degradation and stabilization rates. Different proteins may be bound to the same transcript at different times, conferring different effects on degradation and stabilization.

The RDE can be a Class II AU rich element, and the RNA binding protein can be GAPDH. The Class II AU rich element bound by GAPDH can be AUUUAUUUAUUUA (SEQ ID NO. 14). The Class II AU rich element and GADPH can be used to control the expression of a transgene, e.g., a CAR. The Class II AU rich element and GADPH also can be used to effect the expression of a transgene and/or a CAR in a T-lymphocyte. The Class II AU rich element and GADPH can be used to effect the expression of a transgene and/or a CAR in a CD8+T-lymphocyte. The Class II AU rich element and GADPH can be used to effect the expression of a transgene and/or a CAR in a CD4+T-lymphocyte. The Class II AU rich element and GADPH can be used to effect the expression of a transgene and/or a CAR in a natural killer cell.

The RDE may have microRNA binding sites. The RDE can be engineered to remove one or more of these microRNA binding sites. The removal of the microRNA binding sites can increase the on expression from a construct with an RDE by at least 5, 10, 15, 20, 50 or 100 fold. The RDE with the microRNA sites can be an RDE that is bound by GAPDH. The removal of microRNA sites from the RDE bound by GAPDH can increase the on expression of a construct with the GAPDH sensitive RDE by at least 5-10 fold. This GAPDH control through the RDE can be used to deliver a payload at a target site. The GAPDH control can be tied to activation of the eukaryotic cell by a CAR that recognizes an antigen found preferentially at the target site.

The RDE can be the 3′-UTR of IL-2 or IFN-γ, and removal of micro-RNA sites can increase the rate of expression and/or the dynamic range of expression from a transgene RNA with the RDE. The RDE can be the 3′-UTR of IL-2 and the removed micro-RNA sites can be the MIR-186 sites which deletion increases the kinetics of expression and increases the dynamic range of expression by about 50-fold. The RDE also can be the 3′-UTR of IFN-γ and the micro-RNA sites removed can be the MIR-125 sites.

The dynamic range of expression (control) with an RDE can be increased by optimizing the codons of the transgene controlled by the RDE. By increasing the GC content of the wobble position of the codons in a transgene the efficiency of translation can be increased by 1-2 logs (10-100 fold). The increased efficiency of translation means the amount of expression in the “on” state with the RDE is increased. If the “off” state expression rate is not changed or changed less, the overall dynamic range of control with the RDE is increased.

New RDEs can be obtained from synthetic libraries made by combinatorially mixing and matching parts of known RDEs by applying techniques such as molecular breeding and/or directed evolution to the 3′-UTRs of genes known to have an RDE. For example, multiple 3′-UTRs with different RDEs are fragmented and assembled into synthetic 3′-UTRs that are then screened or selected for RDE activity. RDEs with desired properties can be discovered from such libraries using positive and/or negative selections.

### Chimeric Antigen Receptors

Chimeric antigen receptors (CARs) can be fused proteins comprising an extracellular antigen-binding/recognition element, a transmembrane element that anchors the receptor to the cell membrane and at least one intracellular element. These CAR elements are known in the art, for example as described in patent application US20140242701, which is incorporated by reference in its entirety for all purposes herein. The CAR can be a recombinant polypeptide expressed from a construct comprising at least an extracellular antigen binding element, a transmembrane element and an intracellular signaling element comprising a functional signaling element derived from a stimulatory molecule. The stimulatory molecule can be the zeta chain associated with the T cell receptor complex. The cytoplasmic signaling element may further comprise one or more functional signaling elements derived from at least one costimulatory molecule. The costimulatory molecule can be chosen from 4-1BB (i.e., CD137), CD27 and/or CD28. The CAR may be a chimeric fusion protein comprising an extracellular antigen recognition element, a transmembrane element and an intracellular signaling element comprising a functional signaling element derived from a stimulatory molecule. The CAR may comprise a chimeric fusion protein comprising an extracellular antigen recognition element, a transmembrane element and an intracellular signaling element comprising a functional signaling element derived from a co-stimulatory molecule and a functional signaling element derived from a stimulatory molecule. The CAR may be a chimeric fusion protein comprising an extracellular antigen recognition element, a transmembrane element and an intracellular signaling element comprising two functional signaling elements derived from one or more co-stimulatory molecule(s) and a functional signaling element derived from a stimulatory molecule. The CAR may comprise a chimeric fusion protein comprising an extracellular antigen recognition element, a transmembrane element and an intracellular signaling element comprising at least two functional signaling elements derived from one or more co-stimulatory molecule(s) and a functional signaling element derived from a stimulatory molecule. The CAR may comprise an optional leader sequence at the amino-terminus (N-term) of the CAR fusion protein. The CAR may further comprise a leader sequence at the N-terminus of the extracellular antigen recognition element, wherein the leader sequence is optionally cleaved from the antigen recognition element (e.g., a scFv) during cellular processing and localization of the CAR to the cellular membrane.

### Chimeric Antigen Receptor—Extracellular Element

Exemplary extracellular elements useful in making CARs are described, for example, in U.S. patent application Ser. No. 15/070,352 filed on Mar. 15, 2016, and U.S. patent application Ser. No. 15/369,132 filed Dec. 5, 2016, both of which are incorporated by reference in their entirety for all purposes.

The extracellular element(s) can be obtained from the repertoire of antibodies obtained from the immune cells of a subject that has become immune to a disease, such as for example, as described in U.S. patent application Ser. No. 15/070,352 filed on Mar. 15, 2016, and U.S. patent application Ser. No. 15/369,132 filed Dec. 5, 2016, both of which are incorporated by reference in their entirety for all purposes.

The extracellular element may be obtained from any of the wide variety of extracellular elements or secreted proteins associated with ligand binding and/or signal transduction as described in U.S. patent application Ser. No. 15/070,352 filed on Mar. 15, 2016, U.S. patent application Ser. No. 15/369,132 filed Dec. 5, 2016, U.S. Pat. Nos. 5,359,046, 5,686,281 and 6,103,521, all of which are incorporated by reference in their entirety for all purposes.

The extracellular element can also be obtained from a variety of scaffold protein families which share the common feature of a protein scaffold core with protein loops that can confer binding specificity and which loops can be altered to provide different binding specificities. Knottins are one such scaffold protein that has peptide loops which can be engineered to produce different binding specificities. For example, knottins can be engineered to have high affinity for specific integrin peptides. See for example, Silverman et al., J. Mol. Biol. 385:1064-75 (2009) and Kimura et al, Proteins 77:359-69 (2009), which are incorporated by reference in their entirety for all purposes. Some cancers overexpress certain integrin peptides and such cancers can be targeted by CARs that have an extracellular element that is a knottin specific for the overexpressed integrin. One such integrin is the integrin αvβ6 which is upregulated in multiple solid tumors such as those derived from colon, lung, breast, cervix, ovary/fallopian tubes, pancreas, and head and neck. See for example, Whilding et al., Biochem. Soc. Trans. 44:349-355 (2016), which is incorporated by reference in its entirety for all purposes.

The extracellular element can also be derived from knottins, which are a family of peptides containing a disulfide bonded core that confers outstanding proteolytic resistance and thermal stability. Knottins, which naturally function as protease inhibitors, antimicrobials, and toxins, are composed of several loops that possess diverse sequences amongst family members. Knottins can be engineered to include additional diversity of sequence in the loops to increase and create new binding specificities. Engineered knottins can be made to bind desired targets (e.g., desired antigens) with a desired specificity. Some Knottins bind with nM specificity to integrins and can be used to target a CAR to a certain integrins (e.g., αv3β3/αvβ5, αvβ3/αvβ5/α5β1, or αvβ6 integrins). Integrins such as αvβ6 can be upregulated on solid tumors and so can be suitable targets for a CAR. Such αvβ6 integrin specific CARs can be made using a knottin specific for the αvβ36 integrin as the extracellular element of the CAR. Activation of an engineered cell (e.g., a T-cell) through the αvβ36 knottin-CAR can be used to deliver a pay load to a solid tumor under the control of an RDE that causes expression of the payload upon CAR cell activation.

In an aspect, the extracellular domain can be an antibody or other binding molecule that binds specifically to an onco-sialylated CD43 that is widely found on AML and MDS blasts. See Hazenberg et al., European Hematology Associate abstracts, Abst S511 (2016), which is incorporated by reference in its entirety for all purposes. The antibody AT14-013 binds a specific sialylated epitope on the onco-sialylated CD43 which epitope is not found on CD43 associated with normal cells and tissue. See WO 2016/209079 and WO 2015/093949, both of which are incorporated by reference in their entirety for all purposes. This antibody or antibodies or other binding molecules which compete for onco-sialylated CD43 binding with AT14-013 are used to make anti-onco sialylated CD43 CARs. For example, the variable regions of the heavy and light chain of AT14-013 can be taken and reformatted as a single chain antibody for use as the extracellular domain of a CAR. Such an extracellular domain on a CAR directs the CAR cell (e.g., anti-onco sialylated CD43 CAR T-lymphocyte) to the AML and/or MDS cells targeting them for cell killing or modification by the CAR cell.

In an aspect, the extracellular domain can be an antibody or other binding molecule that binds specifically to complement factor H (CFH), for example, the SCR19 epitope of CFH. Antibodies and CDRs that can be used to make extracellular domains specific for CFH are found, for example, in U.S. Patent Application 20190315842, which is incorporated by reference in its entirety for all purposes. CFH can be aberrantly expressed on many types of solid tumors including, for example, breast cancer, lung cancer, small cell lung cancer, and nonsmall cell lung cancer.

Other tumor associated antigens that can be the target of the CAR include, for example, complement factor H (e.g., lung cancer, breast cancer, other solid tumors), delta opioid receptor (e.g., small cell lung cancer), c-Met (e.g., NSCLC), gpNMB (e.g., melanoma, breast cancer, other solid tumors), TRAP-2 (e.g., epithelial tumors and other solid tumors), CEACAM5 (e.g., colorectal cancer), CD56 (e.g., SCLC), CD25 (e.g., hematological cancers), guanyl cyclase C (e.g., pancreatic cancer), CAG (e.g., solid tumors), LIV-1 (e.g., breast cancer), PTK7 (e.g., lung cancer, colorectal cancer, breast cancer, and ovarian cancer), LAMP-1 (e.g., colorectal cancer, melanoma, laryngeal cancer), P-cadherin 3 (e.g., epithelial tumors), HER-3 (e.g., breast cancer), CD133 (e.g., hepatocellular carcinoma, pancreatic cancer, colorectal cancer, cholangiocarcinoma), GPRC5D (e.g., multiple myeloma), BCMA (e.g., multiple myeloma), CD138 (e.g., multiple myeloma), Ig kappa light chain (e.g., leukemia, lymphoma, NHL, and multiple myeloma), CD30 (e.g., NHL, HD), IL13Ra2 (e.g., glioblastoma), and ligands for NKG2D (e.g., using the NKG2D receptor as the binding domain for, e.g., AML, MDS, and MM).

Other tumor associated antigens that can be the target of the CAR include, for example, mesothelin, disialoganglioside (GD2), Her-2, MUC1, GPC3, EGFRVIII, CEA, CD19, EGFR, PSMA, GPC2, folate receptor p3, IgG Fc receptor, PSCA, PD-L1, EPCAM, Lewis Y Antigen, LiCAM, FOLR, CD30, CD20, EPHA2, PD-1, C-MET, ROR1, CLDN18.2, NKG2D, CD133, TSHR, CD70, ERBB, AXL, Death Receptor 5, VEGFR-2, CD123, CD80, CD86, TSHR, ROR2, CD147, kappa IGG, IL-13, MUC16, IL-13R, NY-ESO-1, IL13RA2, DLL3, FAP, LMP1, TSHR, BCMA, NECTIN-4, MG7, AFP (alpha-fetoprotein), GP100, B7-H3, Nectin-4, MAGE-A1, MAGE-A4, MART-1, HBV, MAGE-A3, TAA, GP100, Thyroglobulin, EBV, HPV E6, PRAME, HERV-E, WTi, GRAS G12V, p53, TRAIL, MAGE-A10, HPV-E7, KRAS G12D, MAGE-A6, CD19, BCMA, CD22, CD123, CD20, CD30, CD33, CD138, CD38, CD7, SLAMF7, IGG FC, MUC1, Lewis Y Antigen, CD133, ROR1, FLT3, NKG2D, Kappa light chain, CD34, CLL-1, TSLP, CD10, PD-L1, CD44V6, EBV, CD5, GPC3, CD56, integrin B7, CD70, MUCL, CKIT, CLDN18.2, TRBC1, TACi, CD56, and CD4.

Still other tumor associated antigens that can be the target of the CAR include, for example, CD2, CD18, CD27, CD37, CD72, CD79A, CD79B, CD83, CD117, CD172, ERBB3, ERBB4, DR5, HER2, CSi, IL-1RAP, ITGB7, SLC2A14, SLC4A1, SLC6A11, SLC7A3, SLC13A5, SLC19A1, SLC22A12, SLC34A1, slc45A3, SLC46A2, Fra, IL-13Ra2, ULBP3, ULBP1, CLD18, NANOG, CEACAM8, TSPAN16, GLRB, DYRK4, SV2C, SIGLEC8, RBMXL3, HISTIHIT, CCR8, CCNB3, ALPPL2, ZP2, OTUB2, LILRA4, GRM2, PGG1, NBIF3, GYPA, ALPP, SPATAi9, FCRLI, FCRLA, CACNG3, UPK3B, 12UMO4, MUC12, HEPACAM, BPI, ATP6VOA4, HMMR, UPK1A, ADGRVI, HERC5, C3AR1, FASLG, NGB, CELSR3, CD3G, CEACAM3, TNFRSFBC, MS4AB, S1PR5, EDNRB, SCN3A, ABCC8, ABCB1, ANO1, KCND2, HTR4, CACNB4, HTR4, CNR2, 26LRB, EXOC1, ENTPPI, ICAM3, ABCGB, SCN4B, SPN, CD68, ITGAL, ITGAM, SCTR, CYYRI, CLCN2, SLARA3, and JAG3.

### Intracellular Element

The intracellular element can be a molecule that can transmit a signal into a cell when the extracellular element of the CAR and/or RDE-CAR (collectively “CARS”) binds to (interacts with) an antigen. The intracellular signaling element can be generally responsible for activation of at least one of the normal effector functions of the immune cell in which the CAR(s) has been introduced. The term “effector function” refers to a specialized function of a cell. Effector function of a T cell, for example, may be cytolytic activity or helper activity including the secretion of cytokines. Thus the term “intracellular signaling element” refers to the portion of a protein which transduces the effector function signal and directs the cell to perform a specialized function. While the entire intracellular signaling domain can be employed, in many cases the intracellular element or intracellular signaling element need not consist of the entire domain. To the extent that a truncated portion of the intracellular signaling domain is used, such truncated portion may be used as long as it transduces the effector function signal. The term intracellular signaling element is thus also meant to include any truncated portion of the intracellular signaling domain sufficient to transduce the effector function signal. Examples of intracellular signaling elements for use in the CARS can include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

Intracellular elements and combinations of polypeptides useful with or as intracellular elements are described, for example, in U.S. patent application Ser. No. 15/070,352 filed on Mar. 15, 2016, and U.S. patent application Ser. No. 15/369,132 filed Dec. 5, 2016, both of which are incorporated by reference in their entirety for all purposes.

### Transmembrane Element and Spacer Element

The CAR, and/or RDE-CAR may comprise a transmembrane element. The transmembrane element can be attached to the extracellular element of CAR, and/or RDE-CAR. The transmembrane element can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). The transmembrane element can be associated with one of the other elements used in the CAR, and/or RDE-CAR. The transmembrane element can be selected or modified by amino acid substitution to avoid binding of such elements to the transmembrane elements of the same or different surface membrane proteins, e.g., to minimize interactions with other members of the receptor complex. The transmembrane element can be modified to remove cryptic splice sites (e.g., CARS made with a CD8 transmembrane domain can be engineered to remove a cryptic splice site) and/or a transmembrane element can be used in the CAR construct that does not have cryptic splice sites. The transmembrane element can be capable of homodimerization with another CAR, and/or RDE-CAR on the cell surface. The amino acid sequence of the transmembrane element may be modified or substituted so as to minimize interactions with the binding elements of the native binding partner present in the same cell.

Transmembrane elements useful in the present invention are described, for example, in U.S. patent application Ser. No. 15/070,352 filed on Mar. 15, 2016, and U.S. patent application Ser. No. 15/369,132 filed Dec. 5, 2016, both of which are incorporated by reference in their entirety for all purposes.

### Receptors

CARS may be used as the receptor with the cell and the RDE-transgene. CARS are described above. In addition to CARS, other receptors may be used to activate or otherwise change conditions in a cell so that a transgene under the control of an RDE is expressed. Receptors that recognize and respond to a chemical signal can be coupled to expression of the transgene through the RDE. For example, ion channel-linked (ionotropic) receptors, G protein-linked (metabotropic) receptors, and enzyme-linked receptors can be coupled to the expression of the transgene.

One class of receptor that can be coupled to transgene expression are immune receptors such as, for example, T-cell receptors, B-cell receptors (aka antigen receptor or immunoglobulin receptor), and innate immunity receptors.

T-cell receptors are heterodimers of two different polypeptide chains. In humans, most T cells have a T-cell receptor made of an alpha (α) chain and a beta (β) chain have a T-cell receptor made of gamma and delta (γ/δ) chains (encoded by TRG and TRD, respectively). Techniques and primers for amplifying nucleic acids encoding the T-cell receptor chains from lymphocytes are well known in the art and are described in, for example, SMARTer Human TCR a/b Profiling Kits sold commercially by Clontech, Boria et al., BMC Immunol. 9:50-58 (2008); Moonka et al., J. Immunol. Methods 169:41-51 (1994); Kim et al., PLoS ONE 7:e37338 (2012); Seitz et al., Proc. Natl Acad. Sci. 103:12057-62 (2006), all of which are incorporated by reference in their entirety for all purposes. The TCR repertoires can be used as separate chains to form an antigen binding domain. The TCR repertoires can be converted to single chain antigen binding domains. Single chain TCRs can be made from nucleic acids encoding human alpha and beta chains using techniques well-known in the art including, for example, those described in U.S. Patent Application Publication No. US2012/0252742, Schodin et al., Mol. Immunol. 33:819-829 (1996); Aggen et al., “Engineering Human Single-Chain T Cell Receptors,” Ph.D. Thesis with the University of Illinois at Urbana-Champaign (2010) a copy of which is found at ideals.illinois.edu/bitsream/handle/2142/18585/Aggen_David.pdf?sequence=1, all of which are incorporated by reference in their entirety for all purposes.

B-cell receptors include an immunoglobulin that is membrane bound, a signal transduction moiety, CD79, and an ITAM. Techniques and primers for amplifying nucleic acids encoding human antibody light and heavy chains are well-known in the art, and described in, for example, ProGen's Human IgG and IgM Library Primer Set, Catalog No. F2000; Andris-Widhopf et al., “Generation of Human Fab Antibody Libraries: PCR Amplification and Assembly of Light and Heavy Chain Coding Sequences,” Cold Spring Harb. Protoc. 2011; Lim et al., Nat. Biotechnol. 31:108-117 (2010); Sun et al., World J. Microbiol. Biotechnol. 28:381-386 (2012); Coronella et al., Nucl. Acids. Res. 28:e85 (2000), all of which are incorporated by reference in their entirety for all purposes. Techniques and primers for amplifying nucleic acids encoding mouse antibody light and heavy chains are well-known in the art, and described in, for example, U.S. Pat. No. 8,143,007; Wang et al., BMC Bioinform. 7(Suppl):S9 (2006), both of which are incorporated by reference in their entirety for all purposes. The antibody repertoires can be used as separate chains in antigen binding domains, or converted to single chain antigen binding domains. Single chain antibodies can be made from nucleic acids encoding human light and heavy chains using techniques well-known in the art including, for example, those described in Pansri et al., BMC Biotechnol. 9:6 (2009); Peraldi-Roux, Methods Mole. Biol. 907:73-83 (2012), both of which are incorporated by reference in their entirety for all purposes. Single chain antibodies can be made from nucleic acids encoding mouse light and heavy chains using techniques well-known in the art including, for example, those described in Imai et al., Biol. Pharm. Bull. 29:1325-1330 (2006); Cheng et al., PLoS ONE 6:e27406 (2011), both of which are incorporated by reference in their entirety for all purposes.

Innate immunity receptors include, for example, the CD94/NKG2 receptor family (e.g., NKG2A, NKG2B, NKG2C, NKG2D, NKG2E, NKG2F, NKG2H), the 2B4 receptor, the NKp30, NKp44, NKp46, and NKp80 receptors, the Toll-like receptors (e.g., TLR1, TLR2, TLR3, TLR4, TLR5, TLR6, TLR7, TLR8, TLR9, TLR10, RP105).

G-protein linked receptors also known as seven-transmembrane domain receptors are a large family of receptors that couple receptor binding of ligand to cellular responses through G proteins. These G-proteins are trimers of α, β, and γ subunits (known as Gα, Gβ, and Gγ, respectively) which are active when bound to GTP and inactive when bound to GDP. When the receptor binds ligand it undergoes a conformational change and allosterically activates the G-protein to exchange GTP for bound GDP. After GTP binding the G-protein dissociates from the receptor to yield a Ga-GTP monomer and a Gβγ dimer. G-protein linked receptors have been grouped together into classes which include, for example, Rhodopsin-like receptors, secretin receptors, metabotropic glutamate/pheromone receptors, fungal mating pheromone receptors, cyclic AMP receptors, and frizzled/smoothened receptors. G-protein receptors are used in a wide variety of physiological processes including detection of electromagnetic radiation, gustatory sense (taste), sense of smell, neurotransmission, immune system regulation, growth, cell density sensing, etc.

Enzyme linked receptors also known as a catalytic receptor, is a transmembrane receptor, where the binding of an extracellular ligand causes enzymatic activity on the intracellular side. Enzyme linked receptors have two domains joined together by a transmembrane portion (or domain) of the polypeptide. The two terminal domains are an extracellular ligand binding domain and an intracellular domain that has a catalytic function. There are multiple families of enzyme linked receptors including, for example, the Erb receptor family, the glial cell-derived neurotrophic factor receptor family, the natriuretic peptide receptor family, the trk neurotrophin receptor family, and the toll-like receptor family.

Ion channel linked receptors also known as ligand-gated ion channels are receptors that allow ions such as, for example, Na⁺, K⁺, Ca²⁺ and Cl⁻ to pass through the membrane in response to the binding of a ligand to the receptor. There are multiple families of ligand-gated ion channels including, for example, cationic cys-loop receptors, anionic cys-loop receptors, ionotropic glutamate receptors (AMPA receptors, NMDA receptors), GABA receptors, 5-HT receptors, ATP-gated channels, and PIP₂-gated channels.

### Eukaryotic Cells

Various eukaryotic cells can be used as the eukaryotic cell. The eukaryotic cells can be animal cells. The eukaryotic cells can be mammalian cells, such as mouse, rat, rabbit, hamster, porcine, bovine, feline, or canine. The mammalian cells can be cells of primates, including but not limited to, monkeys, chimpanzees, gorillas, and humans. The mammalians cells can be mouse cells, as mice routinely function as a model for other mammals, most particularly for humans (see, e.g., Hanna, J. et al., Science 318:1920-23, 2007; Holtzman, D. M. et al., J Clin Invest. 103(6):R15-R21, 1999; Warren, R. S. et al., J Clin Invest. 95: 1789-1797, 1995; each publication is incorporated by reference in its entirety for all purposes). Animal cells include, for example, fibroblasts, epithelial cells (e.g., renal, mammary, prostate, lung), keratinocytes, hepatocytes, adipocytes, endothelial cells, and hematopoietic cells. The animal cells can be adult cells (e.g., terminally differentiated, dividing or non-dividing) or embryonic cells (e.g., blastocyst cells, etc.) or stem cells. The eukaryotic cell also can be a cell line derived from an animal or other source.

The eukaryotic cells can be stem cells. A variety of stem cells types are known in the art and can be used as the eukaryotic cell, including for example, embryonic stem cells, inducible pluripotent stem cells, hematopoietic stem cells, neural stem cells, epidermal neural crest stem cells, mammary stem cells, intestinal stem cells, mesenchymal stem cells, olfactory adult stem cells, testicular cells, and progenitor cells (e.g., neural, angioblast, osteoblast, chondroblast, pancreatic, epidermal, etc.). The stem cells can be stem cell lines derived from cells taken from a subject.

The eukaryotic cell can be a cell found in the circulatory system of a mammal, including humans. Exemplary circulatory system cells include, among others, red blood cells, platelets, plasma cells, T-cells, natural killer cells, B-cells, macrophages, neutrophils, or the like, and precursor cells of the same. As a group, these cells are defined to be circulating eukaryotic cells of the invention. The eukaryotic cell can be derived from any of these circulating eukaryotic cells. Transgenes may be used with any of these circulating cells or eukaryotic cells derived from the circulating cells. The eukaryotic cell can be a T-cell or T-cell precursor or progenitor cell. The eukaryotic cell can be a helper T-cell, a cytotoxic T-cell, a memory T-cell, a regulatory T-cell, a natural killer T-cell, a mucosal associated invariant T-cell, a gamma delta T cell, or a precursor or progenitor cell to the aforementioned. The eukaryotic cell can be a natural killer cell, or a precursor or progenitor cell to the natural killer cell. The eukaryotic cell can be a B-cell, or a B-cell precursor or progenitor cell. The eukaryotic cell can be a neutrophil or a neutrophil precursor or progenitor cell. The eukaryotic cell can be a megakaryocyte or a precursor or progenitor cell to the megakaryocyte. The eukaryotic cell can be a macrophage or a precursor or progenitor cell to a macrophage.

The eukaryotic cells can be obtained from a subject. The subject may be any living organisms. The cells can be derived from cells obtained from a subject. Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. Any number of T cell lines available in the art also may be used. T-cells can be obtained from a unit of blood collected from a subject using any number of techniques known to the skilled artisan, such as Ficoll separation. Cells from the circulating blood of an individual can be obtained by apheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. The cells can be washed with phosphate buffered saline (PBS). In an alternative aspect, the wash solution lacks calcium and may lack magnesium or may lack many if not all divalent cations. Initial activation steps in the absence of calcium can lead to magnified activation.

Enrichment of a T cell population by negative selection can be accomplished with a combination of antibodies directed to surface markers unique to the negatively selected cells. Cells can be enriched by cell sorting and/or selection via negative magnetic immunoadherence or flow cytometry using a cocktail of monoclonal antibodies directed to cell surface markers present on the cells. For example, to enrich for CD4+ cells, a monoclonal antibody cocktail typically includes antibodies to CD14, CD20, CD11b, CD16, HLA-DR, and CD8. It may be desirable to enrich for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005, each of which is incorporated by reference in its entirety for all purposes.

NK cells may be expanded in the presence of a myeloid cell line that has been genetically modified to express membrane bound IL-15 and 4-1BB ligand (CD137L). A cell line modified in this way which does not have MHC class I and II molecules is highly susceptible to NK cell lysis and activates NK cells. For example, K562 myeloid cells can be transduced with a chimeric protein construct consisting of human IL-15 mature peptide fused to the signal peptide and transmembrane domain of human CD8a and GFP. Transduced cells can then be single-cell cloned by limiting dilution and a clone with the highest GFP expression and surface IL-15 selected. This clone can then be transduced with human CD137L, creating a K562-mb15-137L cell line. To preferentially expand NK cells, peripheral blood mononuclear cell cultures containing NK cells are cultured with a K562-mb15-137L cell line in the presence of 10 IU/mL of IL-2 for a period of time sufficient to activate and enrich for a population of NK cells. This period can range from 2 to 20 days, preferably about 5 days. Expanded NK cells may then be transduced with the anti-CD19-BB-ζ (chimeric receptor.

### Pharmaceutical Compositions

Pharmaceutical compositions of the present invention may comprise a CARS and/or transgene-RDE expressing cell, e.g., a plurality of CARS and/or transgene-RDE expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

Suitable pharmaceutically acceptable excipients are well known to a person skilled in the art. Examples of the pharmaceutically acceptable excipients include phosphate buffered saline (e.g. 0.01 M phosphate, 0.138 M NaCl, 0.0027 M KCl, pH 7.4), an aqueous solution containing a mineral acid salt such as a hydrochloride, a hydrobromide, a phosphate, or a sulfate, saline, a solution of glycol or ethanol, and a salt of an organic acid such as an acetate, a propionate, a malonate or a benzoate. An adjuvant such as a wetting agent or an emulsifier, and a pH buffering agent can also be used. The pharmaceutically acceptable excipients described in Remington's Pharmaceutical Sciences (Mack Pub. Co., N.J. 1991) (which is incorporated herein by reference in its entirety for all purposes) can be appropriately used. The composition can be formulated into a known form suitable for parenteral administration, for example, injection or infusion. The composition may comprise formulation additives such as a suspending agent, a preservative, a stabilizer and/or a dispersant, and a preservation agent for extending a validity term during storage.

A composition comprising the eukaryotic cells described herein as an active ingredient can be administered for treatment of, for example, a cancer (blood cancer (leukemia), solid tumor (ovarian cancer) etc.), an inflammatory disease/autoimmune disease (pemphigus vulgaris, lupus erythematosus, rheumatoid arthritis, asthma, eczema), hepatitis, and an infectious disease the cause of which is a virus such as influenza and HIV, a bacterium, or a fungus, for example, a disease such as tuberculosis, MRSA, VRE, or deep mycosis, depending on an antigen to which a CAR, DE-CAR, and/or Side-CAR polypeptide binds.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, intranasally, intraarterially, intratumorally, into an afferent lymph vessel, by intravenous (i.v.) injection, or intraperitoneally. In one aspect, the T cell compositions of the present invention are administered to a patient by intradermal or subcutaneous injection. In one aspect, the T-cell compositions of the present invention are administered by i.v. injection. The compositions of T-cells may be injected directly into a tumor, lymph node, or site of infection. The administration can be done by adoptive transfer.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). A pharmaceutical composition comprising the eukaryotic cells described herein may be administered at a dosage of 10⁴ to 10⁹ cells/kg body weight, in some instances 10⁵ to 10⁶ cells/kg body weight, including all integer values within those ranges. A eukaryotic cell composition may also be administered multiple times at these dosages. Eukaryotic cells can also be administered by using infusion techniques that are commonly known in immunotherapy (see, e.g., Rosenberg et al., New Eng. J. of Med. 319:1676, 1988, which is incorporated by reference in its entirety for all purposes).

### Uses of Eukaryotic Cells

Nucleic acids encoding CARS and/or transgene-RDE(s) can be used to express CAR and/or transgene polypeptides in eukaryotic cells. The eukaryotic cell can be a mammalian cell, including for example human cells or murine cells. The eukaryotic cells may also be, for example, hematopoietic cells including, e.g., T-cells, natural killer cells, B-cells, or macrophages.

T-cells (e.g., CD4+ or CD8+) or natural killer cells can be engineered with a polynucleotide encoding a CAR. The desired amount of effector function can be an optimized amount of effector function with a known amount (and/or density) of target antigen on target cells. Effector function can be target cell killing, activation of host immune cells, cytokine secretion, production of granzymes, production of apoptosis inducing ligands, production of other ligands that modulate the immune system, etc. The effector function can be secretion of cytokines such as, for example, IL-2, IFN-γ, TNF-α, TGF-β, and/or IL-10. Effector function can be the killing of target cells. Target cells can be killed with granzymes. Target cells can be induced to undergo apoptosis. Eukaryotic cells with CARs can kill target cells through apoptosis and granzymes.

The RDE regulatory element can be used to control expression of a transgene. This transgene expression can deliver a payload at a target site. These transgenes can also be carried by viral constructs, or viruses when the payload is a virus. Expression of the transgene can cause a desired change in the eukaryotic cell. An RDE regulated by GAPDH can be used for payload delivery, and the eukaryotic cell (e.g., T-cell, natural killer cell, B-cell, macrophage, dendritic cell, or other antigen presenting cell) can be activated (e.g., by a CAR) when it reaches the target site. Upon activation of the eukaryotic cell at the target site through the CAR, the cell induces glycolysis and the GAPDH releases from the RDE allowed payload expression and delivery. The target site can be a tumor or infection and the transgene could encode a cytokine, a chemokine, an antibody, a checkpoint inhibitor, a granzyme, an apoptosis inducer, complement, an enzyme for making a cytotoxic small molecule, an enzyme that cleaves peptides or saccharides (e.g., for digesting a biofilm), other cytotoxic compounds, or other polypeptides that can have a desired effect at the target site. Checkpoint inhibitors include agents that act at immune checkpoints including, for example, cytotoxic T-lymphocyte-associated antigen 4 (CTLA4), programmed cell death protein (PD-1), Killer-cell Immunoglobulin-like Receptors (KIR), and Lymphocyte Activation Gene-3 (LAG3). Examples of checkpoint inhibitors that may be used as payloads include, for example, Nivolumab (Opdivo®), Pembrolizumab (Keytruda®), Cemiplimab (Libtayo®), Atezolizumab (Tecentriq®), Avelumab (Bavencio®), Durvalumab (Imfinzi®), Ipilimumab (Yervoy@), Lirilumab, and BMS-986016. Nivolumab, Atezolizumab and Pembrolizumab act at the checkpoint protein PD-1 and inhibit apoptosis of anti-tumor immune cells. Some checkpoint inhibitors prevent the interaction between PD-1 and its ligand PD-L1. Ipilimumab acts at CTLA4 and prevents CTLA4 from downregulating activated T-cells in the tumor. Lirilumab acts at KIR and facilitates activation of Natural Killer cells. BMS-986016 acts at LAG3 and activates antigen-specific T-lymphocytes and enhances cytotoxic T cell-mediated lysis of tumor cells.

The payload can be one or more of an anti-IL33 antibody, anti-LAG3 antibody, anti-TIM3 antibody, anti-TIGIT antibody, anti-MARCO antibody, anti-VISTA antibody, anti-CD39 antibody, anti-41BB antibody, IL-15, IL-21, IL-12, CD40L, and/or Leptin. The IL-33 receptor is upregulated in T_{regs} (regulatory T-cells) and anti-IL33 antibody reduces proliferation and activation of T_{regs}. Anti-LAG3 antibody can also decrease activity of T_{regs}. Anti-Il33 antibody and anti-LAG3 antibody can be used alone or together to reduce the activity of T_{regs} which can reduce the suppression of CAR T-cells and other anti-cancer T-cells. Anti-TIM-3 antibody allows co-localization of CD8+ T-cells and DC-1 cells (which improves anti-tumor response). MARCO is expressed on macrophages and in the tumor microenvironment this can be suppressive to T-cells. Anti-MARCO antibody prevents this tumor suppression by macrophages. Anti-VISTA antibody reduces the amount of neutrophils in the tumor microenvironment. A high neutrophil to T-cell ratio in the tumor microenvironment correlates with poor patient outcomes. Decreasing the neutrophils in the tumor can improve patient outcomes and tumor cell killing. IL-15 and 11-21 increase the expansion of natural killer cells and 11-15 can rescue CD8+ T-cells and may prevent T-cell exhaustion. CD40L plays a central role in priming, co-stimulation and activation of T-cells in an immune response. Anti-CD39 antibody can reduce adenosine levels in the tumor microenvironment. High levels of adenosine in the tumor microenvironment can induce immunosuppression. Anti-CD39 antibody can reduce this immunosuppression. Anti-41BB antibody can prevent T-cells from undergoing apoptosis and can also cause tumor cells to upregulate expression of PD1 (so can be combined with anti-PD1 therapies).

Cytokines can include, for example, IL-2, IL-12, IL-15, IL-18, IL-21, IFN-γ, TNF-α, TGF-β, and/or IL-10. IL-18 can include IL-18 variants such as those disclosed herein. Cytotoxic agents can include, for example, granzymes, apoptosis inducers, complement, or a cytotoxic small molecule. The payload can be a gene regulatory RNA, such as, for example, siRNA, microRNAs (e.g., miR155), shRNA, antisense RNA, ribozymes, and the like, or guide RNAs for use with CRISPR systems. The payload can be an anti-4-1BB antibody, anti-CD11b antibody, anti-CTLA4 antibody, anti-IL1b antibody, anti-IL33 antibody, anti-LAG3 antibody, anti-TIM3 antibody, anti-TIGIT antibody, anti-MARCO antibody, anti-VISTA antibody, anti-CD39 antibody, PGC-alpha, Leptin, a BiTE, CCL2, anti-CXCR4 antibody, anti-CXCL12 antibody, HAC, heparinase, hyaluronidase, Hsp60, Hsp70, IL-2, IL-15, IL-18, INFy, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, Ox40-41BB, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), anti-CD28 antibody (including full length and fragments such as single chain antibodies), IL-21, Leptin, GOT2, NAMPT, CD56, IL-2 superkine, anti-REGNASE-1 payloads (e.g., miRNA), C-jun, cysteinase enzyme, cystinase enzyme, PCBP1 (poly(RC) binding protein 1), complement (one or more of B, C1-C9, D, C5b, C3b, C4b, C2a), BMP-1, anti-TGFb agents (e.g., anti-TGFb antibody, soluble TGFbR, anti-avB6 integrin antibody, natural TGFb binding proteins, small molecules such as GW788388, Tranilast, Losartan, HMG CoA reductase inhibitors, Imatinib mesylate, PPAR-g agonists, rosiglitazone, Pirfenidone, Halofuginone), IL7 combined with CCL19 (e.g., IL7-t2A-CCL19), dnTNFR2, dnTGFBR2, DCN, DKK1, OKT3, NOS2, CCL5, anti-4-1BB agonist Antibody, anti-CD11b, anti-CD28 agonist Ab, anti-CD29/anti-VEGF, anti-CTLA4 Ab, anti-IL1b Ab, BiTE, CCR2, CCR4/CXCL12 disruption, HAC, heparinase, HSP60, HSP70, hyaluronidase, IL-12, IL15, IL18 (including IL-18 variants), IL2, anti-CSF1R and anti-IGF1, anti-IL4, IL4 receptor antagonists, IL4 binders (e.g., soluble IL4R), soluble CD40 ligand (e.g., secreted ecto-CD40L), membrane CD40 ligand, a TGFBR antagonist, and/or 4-1BB ligand. An anti-TGFb payloads may be combined with CAR T-cells directed at multiple myeloma (e.g., an anti-GPRC5D CAR). The payloads can also include those found in US20190183932, which is incorporated by reference in its entirety for all purposes. The payload delivered at a target site (e.g., non-tumor target site) can be a factor that protects the target site such as, for example, an anti-inflammatory, a factor that attracts T-regulatory cells to the site, or cytokines or other factors that cause suppression and reduction in immune activity. The payload can be an enzyme that cleaves peptides or saccharides, for example hyaluronidase, heparanase, metalloproteinases and other proteinases which can be used, for example, to digest an undesired biofilm. Myeloid modifying payloads (“MM payloads”) which reduce immune suppression or inhibition caused by myeloid cells may be delivered including, for example, ApoE3, ApoE4, Hsp60, Hsp70, TNFα, antagonists of CSF1 receptor, CD40L (CD154) and/or IL-12. Two or more MM payloads can also be delivered by the CAR, DE-CAR, side-CAR and/or other receptor cell (e.g., T-cell) using RDEs that produce different pharmacokinetics for delivery. For example, the different MM payloads could be controlled by different RDEs so that the Cmax of delivery for the different MM payloads occurs at different times. For example, Myeloid modifying payloads can promote activated M1 macrophages that are proinflammatory and tumoricidal. A MM payload that promotes M1 phenotypes are antagonists of CSF1R (antagonists that block and do not activate the CSF1 receptor and agents that bind CSF1 and prevent it from interacting with the CSF1R). Such antagonists of CSF1R include, for example, small-molecule inhibitors, PLX3397 (Pexidartinib, Plexxikon), PLX7486 (Plexxikon), ARRY-382 (Array Biopharma), JNJ-40346527 (Johnson & Johnson), and BLZ945 (Novartis). Exemplary anubodies which are antagonists of CSF1R include, for example. Emactuzumab (Roche), AMG820 (Angen), IMC-CS4 (LY3022855. Eli Lilly), and MCS110 (Novartis). Cannarile et al, J. Immunotherp. Cancer 5:53 (2017) which is incorporated by reference in its entirety for all purposes. The payload can be localized to the target cell (e.g., tumor site) by fusing or associating the payload with a Small Leucine Rich Proteoglycans (SLRPs) such as Decorin, Biglycan, or fibromodulaon/Lumican. The Decorin, Biglycan, or Lumican can bind to the collagen near the target cell and this binding will localize the payload at or near the target site. This strategy is particularly useful for keeping cytotoxic payloads localized to the target cells (e.g., a tumor). Decorin and Biglycan can also bind to TGF-beta at or near the target site and reduce suppression of the engineered T-cell, and so these can be used as a payload themselves to reduce TGFb. A Decorin, Biglycan, and/or lumican payload can also be constitutively expressed, or expressed under the control of an RDE with a moderate level of baseline expression (mimicking low level constitutive expression coupled with increased expression upon cell activation). The payload can be one or more of any of the above. The payload can be an imaging agent that allows the target site to be imaged. The payload may be a polypeptide that can be imaged directly, or it can be a polypeptide that interacts with a substrate to make a product that can be imaged, imaging polypeptides include, for example, thymidine kinase (PET), dopamine D2 (D2R) receptor, sodium iodide transporter (NIS), dexoycytidine kinase, somatostatin receptor subtype 2, norepinephrine transporter (NET), cannabinoid receptor, glucose transporter (Glutl), tyrosinase, sodium iodide transporter, dopamine D2 (D2R) receptor, modified haloalkane dehalogenase, tyrosinase, β-galactosidase, and somatostatin receptor 2. These reporter payloads can be imaged using, for example, optical imaging, ultrasound imaging, computed tomography imaging, optical coherence tomography imaging, radiography imaging, nuclear medical imaging, positron emission tomography imaging, tomography imaging, photo acoustic tomography imaging, x-ray imaging, thermal imaging, fluoroscopy imaging, bioluminescent imaging, and fluorescent imaging. These imaging methods include Positron Emission Tomography (PET) or Single Photon Emission Computed Tomography (SPECT).

Multiple systems are envisioned for use that can kill target cells directly. These include, for example, the introduction of a viral or a bacterial gene into target cells. This approach turns a non-toxic pro-drug to a toxic one. There are systems that have been extensively investigated: the cytosine deaminase gene (“CD”) of Escherichia coli, which converts the pro-drug 5-Fluorocytosine (“5-FC”) to 5-Fluorouracil (“5-FU”); and the herpes simplex virus thymidine kinase gene (“HSV-tk”), which converts ganciclovir (“GCV”) to ganciclovir monophosphate, converted by the cancer cells' enzymes to ganciclovir triphosphate. The HSV-tk/GCV system useful in killing tumor cells directly, involves adenoviral transfer of HSV-tk to tumor cells, with the subsequent administration of ganciclovir. Specifically, recombinant replication-defective adenovirus is employed to transfer the thymidine, HSV-tk, into hepatocellular carcinoma (“HCC”) cells to confer sensitivity to ganciclovir. Three useful HCC cell lines include, for example, Hep3B, PLC/PRF/5 and HepG2, which can efficiently infect, in vitro, by a recombinant adenovirus carrying lacZ reporter gene (“Ad-CMVlacZ”). Expression of HSV-tk in HCC cells infected with a recombinant adenovirus carrying HSV-tk gene (“AdCMVtk”) induces sensitivity to ganciclovir in a dose-dependent manner (Qian et al., Induction of sensitivity to ganciclovir in human hepatocellular carcinoma cells by adenovirus-mediated gene transfer of herpes simplex virus thymidine kinase, Hepatology, 22:118-123 (1995)) doi.org/10.1002/hep.1840220119.

When the payload is a gene regulatory RNA, such as, for example, siRNAs, shRNAs, and/or microRNAs (e.g., miR155), the regulatory RNA (e.g., mir155) can be the transgene or can be included in an intron of a transgene encoding a polypeptide. For example, a mir155 cassette as described in Du et al., FEBs J. 273:5421-27 (2006) and Chung et al., Nucl Acids Res. 34:e53 (2006) can be used as the payload or be engineered into an intron of a transgene that is used as the payload. The mir155 cassette (or cassette for other regulatory RNA) can be engineered into a transgene as an intron or the transgene can be the mir155 cassette, optionally with additional nucleotides. The regulatory RNA transgene (or transgene with regulatory RNA as an intron) can be placed under the control of an RDE. RDEs can impact RNA processing and stability in the nucleus. After the transgene encoding the regulatory RNA (e.g., mir155) or encoding a transgene with a regulatory RNA (e.g., mir155) intron is transcribed, the transcript can be processed in the nucleus by the nuclear microprocessor complex or other nuclear components to make the nucleotide stem-loop precursor regulatory RNA (e.g., pre-mir155). The pre-regulatory RNA (e.g., pre-mir155) stem-loop is exported out of the nucleus where it is processed by Dicer to form a short RNA duplex. The short RNA duplex(es) are bound by Argonaute (Ago) to form the core of the multi-subunit complex called the RNA-induced silencing complex (RISC). By operably linking a RDE to the transgene encoding the regulatory RNA (e.g., mir155) or the transgene with the regulatory RNA (e.g., mir155) intron, the expression of regulatory RNA (e.g., mir155) can be regulated by the RDE. Different RDEs can be operably linked to the regulatory RNA (e.g., mir155) transgene or transgene with regulatory RNA (e.g., mir155) intron to provide different timing and kinetics of expression following activation of a eukaryotic cell (e.g., activation of a T-cell by the TCR or a CAR). RDEs can be used that produce expression quickly after activation of the cell (e.g., AU2 or AU101), produce high expression 72-96 hours after activation (e.g., AU5 or AU21), or produce increasing expression through 192 hours after expression (e.g., AU19 or AU22). RDEs can also be selected that will produce continuous expression of regulatory RNA (e.g., mir155) or that will produce expression for a period of time after activation of the cell followed by reduced expression. Multiple regulatory RNA (e.g., mir155) constructs (e.g., with mir155 as the transgene or a transgene with a mir155 intron) with different RDEs can be used to provide continuous expression of regulatory RNA (e.g., mir155) following activation of a cell (e.g., T-cell) by using RDEs that provide different pharmacokinetic profiles of expression which together produce continuous expression (e.g., see Example 11). Alternatively, select RDEs or combinations of RDEs or combinations of regulatory RNA (e.g., mir155) with different RDEs can be used to provide a desired expression profile of the regulatory RNA (e.g., mir155).

Upregulation of mir155 has been associated with activated CD8+ T-cells and the formation of memory T-cells after an immunological challenge. Upregulation of mir155 expression during activation of T-cells (e.g., CAR T-cells activated by target antigens) will potentiate the CAR T-cell response against target cells. Placing mir155 under control of a heterologous RDE (e.g., an RDE that responds to GAPDH) ties upregulation of mir155 to activation of the T-cell so that mir155 is upregulated in activated T-cells (e.g., CD8+, CAR T-cells). This upregulation can increase proliferation of activated T-cells. The upregulation can also decrease T-cell exhaustion and senescence. The upregulation can also potentiate T-cell effector functions resulting in increased target cell killing.

Effector function of T-cells can also be enhanced by downregulating TCF7 and/or Tox expression and/or by upregulating IL-15 expression. TCF7 is a member of the T-cell factor/lymphoid enhancer-binding factor family of high mobility group (HMG) box transcriptional activators. This gene is expressed predominantly in T-cells and plays a critical role in natural killer cell and innate lymphoid cell development. HMG box protein TCF7 can be a regulator in the switch between self-renewal and differentiation. TCF7 can have a dual role in promoting the expression of genes characteristic of self-renewing CD34+ cells while repressing genes activated in partially differentiated CD34− state. TCF7 can regulate a network of transcription factors that switch cells from a naïve, undifferentiated state to a differentiated, effector cell state. When TCF7 is expressed cells adopt a self-renewal state that is more naïve and less differentiated. TCF7 can be downregulated using miRNAs such as, for example, mIR-192, mIR-34a, miR-133a, miR-138-5p, miR-342-5p, miR-491-5p, and/or miR-541-3p. In an example, one of more of these miRNAs can be encoded in one or more introns of a payload that are spliced out when the transcript is bound by hnRNPLL (see above), and when the payload is expressed in an activated cell making hnRNPLL these miRNAs will downregulate TCF7. Alternatively, a transgene encoding TCF7 can be used as an off switch for activated CAR T-cells. If TCF7 is expressed after the effector, CAR T-cell has killed the target cancer cells, this should push the CAR T-cell into a naïve, undifferentiated state (an off state for the CAR T-cell). The transgene encoding TCF7 could be placed under the control of an inducible promoter (e.g., an inducible promoter that is ligand inducible) or it could be placed under control of an RDE that results in expression after eight days or more of cell activation (e.g., see Example 11). Expression of TCF7 can be turned off by removal of ligand (or other inducing factors for the inducible promoter), and/or the RDE control will turn off expression. This can return the CAR T-cell to state where it can be reactivated by binding to target ligand at other cancer cells.

Thymocyte selection-associated high mobility group box (TOX) protein is a member of a small subfamily of proteins (TOX2, TOX3, and TOX4) that share almost identical HMG-box sequences. TOX can be induced by high antigen stimulation of the T cell receptor and TOX can be a central regulator of T_{EX} (exhausted T-cells). Robust TOX expression can result in commitment to development of the T_{EX} cell type. TOX exhaustion may counteract and balance T-cell overstimulation and activation-induced cell death in settings of chronic antigen stimulation. Effector T-cells (e.g., activated CD8+ T-cells) can have low Tox, whereas higher levels of Tox pushes the effector cells to become T_{EX} cells. T_{EX} cells have reduced effector function but are still effective against low level infections or small numbers of cancer cells.

Effector function of T-cells can be enhanced by including a payload encoding an miRNA for Tox (e.g., hsa-mir-26b-5p (MIRT030248) hsa-mir-223-3p (MIRT054680)) under regulation of an RDE. Following activation of the T-cell, the RDE control will result in expression of the miRNA for Tox. This miRNA will lower levels of Tox in the T-cell inhibiting T_{EX} formation by the activated T-cells resulting in more active, effector T-cells against a target. In addition, a payload can be Tox itself, used as on off-switch that pushes the activated T-cells into a T_{EX} phenotype at a desired time. When used as an off-switch, Tox expression can be under control of an inducible promoter that can be induced to express Tox at a desired time (e.g., by adding an appropriate ligand), Tox can be controlled by an RNA control device or a DE (ligand can induce expression), or Tox can be placed under control of an RDE that produces expression at late time intervals after activation of the cell (e.g., see Example 11). Functional state and type of T-cell can tailored by treating T-cells with electromagnetic radiation. Electromagnetic radiation in the UV range can condition T-cells to become Treg cells. For example, a dose of UVA/UVB can induce formation of Tregs. Electromagnetic radiation in the blue light range can activate T-cells.

An exemplary payload is a transgene encoding ApoE (e.g., ApoE2, ApoE3 and/or APoE4) which is secreted from the cell. ApoE can bind to receptors (e.g., LRP8) on Myeloid Derived Suppressor Cells (MDSC) and reduce the survival of MDSCs. MDSCs are a heterogeneous population of suppressive innate immune cells that can expand in certain disease states. In some cancers (e.g., melanoma, lung, breast and ovarian cancers) MDSC levels can substantially rise in the tumor(s) and in the plasma of patients. Such patients with high levels of circulating MDSCs can respond poorly to checkpoint blockade. MDSCs can mediate immunosuppression in these patients and induce angiogenesis. Payload expression of ApoE (e.g., ApoE4) can reduce the number of MDSCs in tumors and circulating in the serum, and result in suppression of tumor progression and metastatic colonization. The reduction in MDSCs in the tumor also enables other immune cells (e.g., CAR T-cells) to more efficiently kill tumor cells. The ApoE payload can also act directly on myeloid malignancies that express the LRP8 receptor. In such examples, the payload delivery of ApoE to a myeloid cancer cell can suppress and/or kill the cancer cell. Thus, ApoE can be a payload for delivery to myeloid malignancies that are LRP8+, including LRP8+ AML. Delivery of the ApoE payload by a eukaryotic cell (e.g., primary T-cell) can be combined with another therapeutic agent such as, for example, an anti-cancer agent (e.g., a CAR T-cell, a chemotherapeutic, radiation, a checkpoint inhibitor, or any of the anti-cancer therapeutics described herein). The ApoE effect on MDSCs can potentiate the action of the other anti-cancer agent.

Another exemplary payload is a transgene encoding NO-synthase (e.g., iNOS, nNOS and eNOS). NO synthase can bind to GAPDH and can sequester the GAPDH allowing RDE (which are bound by GAPDH) controlled transgenes (or native genes) to be expressed, or increasing expression from RDE (which are bound by GAPDH, e.g., AU 19 (TMEM-219), AU 20 (TMEM-219snp), AU 21 (CCR7), AU 22 (SEM-A4D), and AU 23 (CDC42-SE2)) controlled transgenes (or native genes) once the cell is activated and glycolysis is induced. Expression of NO synthase can induce RDE (which are bound by GAPDH) controlled expression (through binding to GAPDH) and/or can potentiate RDE (which are bound by GAPDH) controlled expression by decreasing the amount of GAPDH that can bind RDEs and/or increasing the time over which GAPDH cannot bind to RDEs. When NO synthase is used to increase the RDE (which are bound by GAPDH) response to cell activation, a transgene encoding NO synthase can be placed under control of an RDE so that when the cell is activated, expression from the transgene encoding the NO synthase is induced. When NO synthase is used to induce expression from RDE (which are bound by GAPDH) controlled genes, the NO synthase can be placed under inducible control (e.g., inducible promoters, RNA control devices, or destabilizing elements as disclosed in U.S. Pat. No. 9,777,064, which is hereby incorporated by reference in its entirety for all purposes) and induction of NO synthase expression induces expression from the RDE (which are bound by GAPDH) controlled genes.

An exemplary payload is a transgene encoding HSV-Thymidine Kinase (HSV-TK). HSV-TK can be used as an adjuvant, and/or as a super antigen that induces an inflammatory response in the patient. When used in this manner, a cell secretes the HSV-TK payload at the target site inducing an inflammatory response. The transgene encoding the HSV-TK can also be used as a kill switch to eliminate the engineered cells (e.g., CAR T-cells with or without a RDE controlled payload). When used as a kill switch, the HSV-TK can be controlled by a late expressing RDE so the HSV-TK is expressed after the CAR T-cell has acted at the target site, or the transgene expressing the HSV-TK can be controlled by a ligand inducible control means so that the HSV-TK protein is expressed in response to the ligand which is introduced at a desired time. In the kill-switch application, ganciclovir can be provided to the cells and the HSV-TK converts the ganciclovir to GCV-triphosphate which kills the cell by a cytotoxic effect. A transgene expressing HSV-TK can also be included in a viral payload so that when the virus infects target cells the target cells express HSV-TK. Ganciclovir is provided to the target cells which use the HSV-TK to convert the ganciclovir to GCV-triphosphate which is toxic to the target cells.

A eukaryotic cell can bind to a specific antigen via the CAR, -cell receptor, or other receptor to transmit a signal into the eukaryotic cell, and as a result, the eukaryotic cell can be activated and so express an appropriate RDE-transgene. The activation of the eukaryotic cell expressing the CARS is varied depending on the kind of a eukaryotic cell and the intracellular element of the CARS. The eukaryotic cell can express a RDE transcript that poises the cell for effector function upon stimulation of the eukaryotic cell through a CARS.

A eukaryotic cell expressing the RDE-transgene or RDE transcript, and optionally, a CARS, T-cell receptor, B-cell receptor, innate immunity receptor and/or other receptor or targeting polypeptide can be used as a therapeutic agent to treat a disease. The therapeutic agent can comprise the eukaryotic cell expressing the RDE-transgene or RDE transcript, and optionally, a CARS, T-cell receptor, B-cell receptor, innate immunity receptor and/or other receptor or targeting polypeptide as an active ingredient, and may further comprise a suitable excipient. Examples of the excipient include pharmaceutically acceptable excipients for the composition. The disease against which the eukaryotic cell expressing the RDE-transgene or RDE transcript, and optionally, a CARS, T-cell receptor, B-cell receptor, innate immunity receptor and/or other receptor or targeting polypeptide is administered is not particularly limited as long as the disease shows sensitivity to the eukaryotic cell and/or the product of the RDE-transgene.

Examples of diseases that can be treated include a cancer (blood cancer (leukemia), solid tumor (ovarian cancer) etc.), an inflammatory disease/autoimmune disease (asthma, eczema), hepatitis, and an infectious disease, the cause of which is a virus such as influenza and HIV, a bacterium, or a fungus, for example, tuberculosis, MRSA, VRE, and deep mycosis, other immune mediated diseases such as neurodegenerative diseases like Alzheimer's or Parkinson's, and metabolic diseases like diabetes. A receptor (e.g., a CAR) can target the eukaryotic cell to the diseased cell(s) and when the receptor binds to its target at the diseased cell(s) the receptor can send a signal into the eukaryotic cell leading to expression of the RDE-transgene. The RDE-transgene encodes a polypeptide that is useful in treating or killing the diseased cell(s). A cancer and/or solid tumor can be treated with a eukaryotic cell expressing receptor that binds to a tumor associated (or cancer associated) antigen, such as those described above. When the receptor binds to the tumor associated antigen the receptor sends a signal into the cell that causes the RDE-transgene to be expressed (e.g., the signal effects an RDE binding protein leading to expression of the RDE-transcript). The RDE-transcript can encode a polypeptide that activates the eukaryotic cell so that the eukaryotic cell treats the cancer and/or the RDE-transcript encodes a polypeptide that itself treats the cancer (e.g., a cytotoxic polypeptide).

An autoimmune disease (e.g., pemphigus vulgaris, lupus erythematosus, rheumatoid arthritis, multiple sclerosis, Crohn's disease) can be treated with a eukaryotic cell expressing a RDE-transgene or RDE transcript, and optionally, a CARS, T-cell receptor, B-cell receptor, innate immunity receptor and/or other receptor or targeting polypeptide that binds to the immune proteins associated with the autoimmune disease. The receptor or targeting polypeptide can trigger expression of the RDE-transgene that encodes a polypeptide useful in treating the autoimmune disease (e.g., the polypeptide can regulate the cells causing the autoimmune disease or kill these cells). The eukaryotic cell expressing the RDE-transgene or RDE transcript, and receptor or targeting polypeptide can target cells that make an antibody involved with the autoimmune disease (e.g., the RDE-transgene can encode a polypeptide that kills the antibody producing cells or that inhibits the production of antibody by these cells). The eukaryotic cell expressing the RDE-transgene or RDE transcript, and receptor or targeting polypeptide can target T-lymphocytes involved with the autoimmune disease (e.g., the RDE-transgene can encode a polypeptide that kills the target T-lymphocytes or that regulates the activity of the T-lymphocytes).

The therapeutic agent comprising the eukaryotic cell expressing the CARS, T-cell receptor, B-cell receptor, innate immunity receptor and/or other receptor or targeting polypeptide as an active ingredient can be administered intradermally, intramuscularly, subcutaneously, intraperitoneally, intranasally, intraarterially, intravenously, intratumorally, or into an afferent lymph vessel, by parenteral administration, for example, by injection or infusion, although the administration route is not limited.

The RDE-transgene or RDE transcript, and optionally, CARS, T-cell receptor, B-cell receptor, innate immunity receptor and/or other receptor or targeting polypeptide can be used with a T-lymphocyte that has aggressive anti-tumor properties, such as those described in Pegram et al, CD28z CARs and armored CARs, 2014, Cancer J. 20(2):127-133, which is incorporated by reference in its entirety for all purposes. The RDE transcript can encode a polypeptide that causes aggressive anti-tumor properties in the T-lymphocyte.

A transgene, a CAR polypeptide or other transgene can be controlled by an RDE from the 3′-UTR of the gene encoding IL-2 or the 3′-UTR of IFN-γ. These RDEs can be modified to inactivate microRNA sites found in the RDE. Using these control elements makes expression of the CAR, DE-CAR, Side-CAR, and/or transgene sensitive to changes in the glycolytic state of the host cell through the interaction of the RDE with glyceraldehyde-3-phosphate dehydrogenase (GAPDH). When the host cell is in a quiescent state a large proportion of the GAPDH is not involved in glycolysis and is able to bind to the RDE resulting in reduced translation of the transcript encoding the CAR, DE-CAR, Side-CAR, and/or transgene polypeptides. When the host cell is induced to increase glycolysis, e.g., by providing the host cells with glucose, or other small molecules that will increase glycolytic activity, GAPDH becomes enzymatically active and is not able to bind to the RDE. The reduction in GAPDH binding to the RDE increases translation of the transcripts (e.g., by increasing half-life of the transcript and/or by increasing the translation rate) encoding the CAR, DE-CAR, Side-CAR, or other transgene. The glycolytic activity of GAPDH can be increased by increasing the amount and/or activity of triose isomerase. The host cell can be induced to over-express a recombinant triose isomerase, and this over-expression increases the glycolytic activity of GAPDH. A glycolysis inhibitor can be added to decrease expression of the transcript with the RDE. Such glycolysis inhibitors include for example, dimethylfumarate (DMF), rapamycin, 2-deoxygicose, 3-bromophyruvic acid, iodoacetate, fluoride, oxamate, ploglitazone, dichloroacetic acid, quinones (e.g., chloroquine, hydroxychloroquine, etc.), or other metabolism inhibitors such as, for example, dehydroepiandrosterone. Expression from the RDE controlled transcript can be increased by the addition of GAPDH (or other RDE binding protein) inhibitor that inhibits binding of the RDE by GAPDH (or other RDE binding protein). Such GAPDH inhibitors include, for example, CGP 3466B maleate or Heptelidic acid (both sold by Santa Cruz Biotechnology, Inc.), pentalenolactone, or 3-bromopyruvic acid. Quinones such as, for example, chloroquine and hydroxychloroquine, can de-acidify the endosome impairing antigen processing by APCs, decrease signaling from toll-like receptors, reduces T-cell proliferation, T-cell metabolic activity, T-cell cytokine secretion, interferes with IL-2 production, and interferes with T-cell response to IL-2.

RDEs can be used to reduce CAR expression in immune cells until those immune cells are activated by target or at a desired time. This can result in expression of the CAR at desired times for therapeutic effect while reducing the systemic exposure of a subject to the CAR. The reduced systemic exposure can reduce and/or inhibit the development of an immune response against the CAR as the subject's immune system will see less CAR over time.

Control of receptor (e.g., CAR and/or TCR) expression can be used to modulate the PK-PD axis of an immunotherapy. The amount of receptor expressed on the surface of cell can be modulated with the strength of a promoter, the inducibility of the promoter, the use of bicistronic constructs with different promoter strengths expressing the two cistrons, RDEs (selection of RDE impacts dynamic range and timing of expression), GC3 content of the transcript, RNA control devices, degrons and/or Side-CARs. These control elements used singly or in combination change the amount of receptor on the surface of the cell which changes the input signal (e.g., amount of ligand for the receptor) needed to activate the cell so that it produces an output (e.g., payload delivery or target cell killing). Using this control, the input signal needed for the receptor cells can be optimized for a given target, compartment of the body, reduction of side effects, etc. as desired. RDEs can also be used to change the timing of the output from the cell after activation at the receptor (e.g., CAR and/or TCR).

Some neural degenerative diseases and syndromes are associated with inflammation, as are a number of other non-neural diseases and syndromes. Such inflammation associated diseases can be treated, at least in part, by providing a subject with small molecules (or other molecules) that increase the availability of inhibitory RDE binding proteins within immune cells. Such small molecules include, for example, glycolysis inhibitors (e.g., dimethylfumarate (DMF), rapamycin, 2-deoxygducose, 3-bromophyruvic acid, iodoacetate, fluoride, oxamate, ploglitazone, dichloroacetic acid), other metabolic inhibitors (e.g., dehydroepiandrosterone), etc. For example, glycolytic inhibitors reduce glycolysis in the cell and can increase the amount of free GAPDH (not involved in glycolysis) for binding to RDEs reducing the expression of these transcripts. A number of inflammatory gene products in immune cells (e.g., gene products that activate the immune system) are regulated by RDEs that can bind GAPDH. Decreasing glycolysis increases the amount of free GAPDH for RDE binding, increases the amount of GAPDH bound to the RDEs of these inflammatory genes and reduces the expression of these inflammatory genes. Inflammatory genes include proinflammatory cytokines such as, for example, IL-1, TNF-α, INF-g, and GM-CSF. These cytokines have 3′-UTRs with RDEs that can bind RDE binding proteins, including GAPDH, to regulate their expression. The increased GAPDH can bind to these RDEs and decrease the expression of these proinflammatory cytokines. Reduced expression of proinflammatory cytokines could reduce activity of the immune system in these subjects reducing inflammation. The reduction in inflammation can have positive therapeutic effects alleviating symptoms and/or treating the underlying disease state in these inflammation related neural diseases, as well as in other inflammation associated diseases and syndromes.

RDEs (e.g., AU elements) can be selected to provide maximal expression at a desired time point and to provide a desired amount of polypeptide at that time point. RDEs can also be selected to provide a desired area under the curve for a polypeptide. As shown in Table 2 of Example 20, different RDEs (e.g., AU elements) reached maximal rates of expression at different times. Also as shown in Table 1, different RDEs provided different amounts of expression with different profiles over time providing different AUC. Using these RDEs in combination with different transgenes allows temporal programming of when the different transgenes reach maximal rates of expression in relation to one another following activation of a cell. In addition, using different RDEs one can program the transgenes to express a desired amount of transgene encoded polypeptide and/or a desired amount of AUC or exposure to the polypeptide encoded by the transgene. Thus, RDEs can be used to provide control that produces desired amounts of different transgene polypeptides at a different (or the same) desired times.

This temporal control can be used to provide desired timing for the production of different transgene polypeptides within a cell. Using this temporal control, a cell can be programmed to express a first transgene that alters the state of the cell so that is prepared to be affected by the polypeptide of a second transgene that is expressed at a later time. For example, the first expressed polypeptide could induce the cell to make and store cytotoxic polypeptides (e.g., granzymes and/or perforins) and the second expressed polypeptide could be involved in the release of the cytotoxic polypeptides. Another example of temporal expression involves it use to program a cell to undergo changes (e.g., differentiation or changing a state of the cell) that requires temporal expression of two or more gene products. RDEs can be used to mimic this temporal expression allowing one to control when the cell changes its state or differentiates (e.g., programmed differentiation of stem cells). In a stem cell example, the temporal and induction control can be used to program a stem cell to differentiate when (and where) it is desired to have the stem cell differentiate into a desired cell type.

The temporal control can also be used to provide desired timing of the production of different transgene polypeptides outside of the cell. Using this temporal control, a cell can be activated and secrete a first transgene polypeptide that conditions and/or alters a target cell so that the target cell is prepared to be acted upon by a polypeptide expressed at later time from a second transgene. For example, the first polypeptide could induce a target cell to express a receptor on the target cell surface (e.g., FasR, Her2, CD20, CTLA-4, PD-L1, etc.) or a polypeptide in the cell. The first transgene could also induce the cell to secrete a factor that induces the target cell to change its state (e.g., the first transgene could induce the cell to secrete CpG which causes the target cell to express OX40 on the target cell surface). The second transgene that reaches maximal rate of expression at a later time can encode a polypeptide that acts on the induced surface receptor (e.g., FasL, Herceptin, Rituximab, Ipilimumab, Nivolumab, anti-OX40 antibody, etc.). The temporal and induction control can also be used to change the state or differentiation of a target cell by providing to the target cell polypeptides in a timed manner where the first polypeptide induces the target cell to alter its state (e.g., differentiation) so that it can be acted upon by the second polypeptide (etc. for additional transgene polypeptides which reach maximal rate of expression at later times).

Autoimmune diseases and other disease states involving an overactive immune system (e.g., SARS-CoV-2 infection) can be treated with a ΔITAM CAR T-cell targeted against autoimmune disease antigen(s). The ΔITAM CAR T-cell can include a payload of IL-4, IL-10 or other immunosuppressive. The ΔITAM CAR T-cell with or without a payload can induce the formation of Tregs that can inhibit the autoimmune disease and/or reduce the toxicity caused by over-stimulation or chronic stimulation of the immune system.

Some examples of diseases and payloads that can be treated using RDEs (Gold elements) with different kinetic parameters (e.g., an RDE that gives rapid expression early after activation of the cell followed by a rapid decline in expression or an RDE that delays expression after cell activation for 2-3 days) include the following: DLL3 positive cancers (such as IDH1mut gliomas, melanoma, and SCLC) using an anti-DLL3 CAR and a payload of one or more of anti-4-1BB antibody, anti-CD11b antibody, anti-CTLA4 antibody, anti-ILlb antibody, a BiTE, CCL2, anti-CXCR4 antibody, anti-CXCL12 antibody, HAC, heparinase, hyaluronidase, Hsp60, Hsp70, IL-2, IL-12, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-DLL3 CAR with an RDE controlled payload is combined or administered in succession with another therapy as described above. The combined or sequenced therapy can be an ADC where the antibody binds to a tumor associate antigen, e.g., DLL3. The combination therapy can be provided to a subject prior to, at the same time, or after the administration of the anti-DLL3 CAR with an RDE controlled payload. CD19 positive lymphomas (e.g., NHL) using an anti-CD19 CAR and a payload of IL-12, or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-CD19 CAR with an RDE controlled payload is combined or administered in succession with another therapy as described above. The combined or sequenced therapy can be an ADC where the antibody binds to a tumor associate antigen, e.g., CD19. The combination therapy can be provided to a subject prior to, at the same time, or after the administration of the anti-CD19 CAR with an RDE controlled payload. AML with onco-CD43 (sialylation mutant) using an anti-onco-CD43 CAR that recognizes the mutated sialylation and a payload of one or more of anti-CXCL12 antibody, anti-anti-CXCR4 antibody, or IL-12, and/or one or more of anti-4-1BB antibody, anti-CD11b antibody, anti-CTLA4 antibody, anti-IL1b antibody, a BiTE, CCL2, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-onco-CD43 CAR with an RDE controlled payload is combined or administered in succession with another therapy as described above. The combined or sequenced therapy can be an ADC where the antibody binds to a tumor associated antigen, e.g., onco-CD43. The combination therapy can be provided to a subject prior to, at the same time, or after the administration of the anti-onco-CD43 CAR with an RDE controlled payload. PSCA positive prostate cancer, bladder cancer or pancreatic cancer using an anti-PSCA CAR and a payload of heparinase or IL-12, and/or one or more of anti-4-1BB antibody, anti-CD11b antibody, anti-CTLA4 antibody, anti-ILlb antibody, a BiTE, CCL2, anti-CXCR4 antibody, anti-CXCL12 antibody, HAC, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-PSCA CAR with an RDE controlled payload is combined or administered in succession with another therapy as described above. The combined or sequenced therapy can be an ADC where the antibody binds to a tumor associated antigen, e.g., PSCA. The combination therapy can be provided to a subject prior to, at the same time, or after the administration of the anti-PSCA CAR with an RDE controlled payload. Triple negative breast cancer with a CAR that recognizes cancer testis antigen, misfolded or mutant EGFR (associated with triple negative breast cancer), and/or folate receptor alpha peptide and a payload of IL-12 and/or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-cancer testis antigen CAR, anti-misfolded or mutant EGFR CAR, or anti-folate receptor alpha CAR with an RDE controlled payload is combined or administered in succession with another therapy as described above. The combined or sequenced therapy can be an ADC where the antibody binds to a tumor associated antigen, e.g., cancer testis antigen, misfolded or mutant EGFR (associated with triple negative breast cancer), and/or folate receptor alpha peptide. The combination therapy can be provided to a subject prior to, at the same time, or after the administration of the anti-cancer testis antigen CAR, anti-misfolded or mutant EGFR CAR, or anti-folate receptor alpha CAR with an RDE controlled payload. SEZ6 positive small cell lung cancer (SCLC), neuroendocrine cancers (e.g., medullary thyroid cancer), large cell lung cancer (LCLC), and malignant pheochromocytoma with a CAR that recognizes SEZ6 and a payload of IL-12 and/or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-SEZ6 CAR with an RDE controlled payload is combined or administered in succession with another therapy as described above. The combined or sequenced therapy can be an ADC where the antibody binds to a tumor associated antigen, e.g., SEZ6. The combination therapy can be provided to a subject prior to, at the same time, or after the administration of the anti-SEZ6 CAR with an RDE controlled payload. RNF43 positive colorectal cancer, colon cancer, and endometrial cancers with a CAR that recognizes RNF43 and a payload of IL-12 and/or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-RNF43 CAR with an RDE controlled payload is combined or administered in succession with another therapy as described above. The combined or sequenced therapy can be an ADC where the antibody binds to a tumor associated antigen, e.g., RNF43. The combination therapy can be provided to a subject prior to, at the same time, or after the administration of the anti-RNF43 CAR with an RDE controlled payload. TnMUC1 positive breast cancer or pancreatic cancer with a CAR that recognizes TnMUC1 and a payload of IL-12 and/or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-TnMUC1 CAR with an RDE controlled payload is combined or administered in succession with another therapy as described above. The combined or sequenced therapy can be an ADC where the antibody binds to a tumor associated antigen, e.g., TnMUC1. The combination therapy can be provided to a subject prior to, at the same time, or after the administration of the anti-TnMUC1 CAR with an RDE controlled payload. Nectin4 positive urothelial cancer, NSCLC, breast cancer, ovarian cancer, bladder cancer, pancreatic cancer, and other solid tumors with a CAR that recognizes Nectin4 and a payload of IL-12 and/or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-Nectin4 CAR with an RDE controlled payload is combined or administered in succession with another therapy as described above. The combined or sequenced therapy can be an ADC where the antibody binds to a tumor associated antigen, e.g., Nectin4. The combination therapy can be provided to a subject prior to, at the same time, or after the administration of the anti-Nectin4 CAR with an RDE controlled payload. EFNA4 positive triple negative breast cancer, ovarian cancer, colorectal cancer, liver cancer, lung cancer, and other solid tumors with a CAR that recognizes EFNA4 and a payload of IL-12 and/or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-EFNA4 CAR with an RDE controlled payload is combined or administered in succession with another therapy as described above. The combined or sequenced therapy can be an ADC where the antibody binds to a tumor associated antigen, e.g., EFNA4. The combination therapy can be provided to a subject prior to, at the same time, or after the administration of the anti-EFNA4 CAR with an RDE controlled payload. GPC3 positive hepatocellular carcinoma, lung cancer and other solid tumors with a CAR that recognizes GPC3 and a payload of IL-12 and/or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies). Optionally, the anti-GPC3 CAR with an RDE controlled payload is combined or administered in succession with another therapy as described above. The combined or sequenced therapy can be an ADC where the antibody binds to a tumor associated antigen, e.g., GPC3. The combination therapy can be provided to a subject prior to, at the same time, or after the administration of the anti-GPC3 CAR with an RDE controlled payload. Complement factor H (CFH) positive breast cancer, lung cancer, nonsmall cell lung cancer (NSCLC), small cell lung cancer (SCLC), and other solid tumors with a CAR that recognizes CFH and a payload of IL-12 and/or one or more of anti-4-1BB antibody, anti-CD11b antibody, anti-CTLA4 antibody, anti-IL1b antibody, a BiTE, CCL2, anti-CXCR4 antibody, anti-CXCL12 antibody, HAC, heparinase, hyaluronidase, Hsp60, Hsp70, IL-2, IL-15, IL-18, IL-18 variants (e.g., SEQ ID NO: 3-4), INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), anti-CD28 antibody (including full length and fragments such as single chain antibodies) and/or anti-TGFb agents (e.g., anti-TGFb antibody, soluble TGFbR, anti-avB6 integrin antibody, natural TGFb binding proteins, dnTGFBR2, biglycan, decorin). Optionally, the anti-CFH CAR with an RDE controlled payload is combined or administered in succession with another therapy as described above. The combined or sequenced therapy can be an ADC where the antibody binds to a tumor associated antigen, e.g., CFH. The combination therapy can be provided to a subject prior to, at the same time, or after the administration of the anti-CFH CAR with an RDE controlled payload.

In general, any of the above CAR cells with or without an RDE controlled transgene(s) can be used in combination or administered in succession with another molecule (e.g., another therapy). For example, the other molecule can be a polypeptide, lipid, carbohydrate, nucleic acid, small molecule drug, antibody, antibody-drug-conjugate, biological drug, or any combination of the foregoing. The antibody drug conjugate (ADC) includes those described herein. The ADC can bind to the same antigen as the CAR or it can bind to a different antigen. When the ADC and CAR bind to the same antigen, they may bind to the same or different epitopes on the same antigen. The ADC and CAR therapy (with or without a RDE controlled payload) can be provided at the same time, or one can be administered to a subject before the other. For example, the ADC and CAR can target a tumor associate antigen and the ADC can be administered the subject first to reduce the tumor burden, and then the CAR therapy is administered to clear the remaining cancer cells.

### Combination Therapies

This disclosure provides compositions and methods for providing a CAR T-lymphocyte expressing a transgene under the control of an RDE in combination or in an order of succession with another therapy. The other therapy can include, for example, a chemotherapeutic, an antibody, and antibody-drug conjugate, a radiotherapy, an alkylating agent, a plant alkaloid, an antitumor antibiotic, an antimetabolite, a topoisomerase inhibitor, and/or an anti-neoplastic. For example, the other therapy can be an antibody drug conjugate that has the same or different specificity as the CAR T-lymphocyte.

Antibodies and antibody-drug conjugates (ADC) can bind to a tumor associated antigen, including, for example, any of the tumor associate antigens described herein as targets for a CAR. The drug component of the ADC can be, for example, a chemotherapeutic, a radionucleotide, an alkylating agent, a plant alkaloid, an antitumor antibiotic, an antimetabolite, a topoisomerase inhibitor, and/or an anti-neoplastic. The drug component of the ADC can be attached to the antibody through a linker which can be cleavable or non-cleavable in nature.

Alkylating agents can include, for example, mustard gas derivatives (e.g., mechlorethamine, cyclophosphamide, chlorambucil, melphalan, or ifosfamide), ethylenimines (e.g., thiotepa or hexamethylmelamine), alkylsulfonates (e.g., busulfan), hydrazines and triazines (e.g., altretamine, procarbazine, dacarbazine, or temozolomide), nitrosoureas (e.g., carmustine, lomustine or streptozocin), and metal salts (e.g., carboplatin, cisplatin, or oxaliplatin). Plant alkaloids can include, for example, Vinca alkaloids (e.g., vincristine, vinblastine, or vinorelbine), taxanes (e.g., paclitaxel or docetaxel), podophyllotoxins (e.g., etoposide or tenisopide), and camptothecan analogs (e.g., irinotecan or topotecan). Antitumor antibiotics can include, for example, anthracyclines (e.g., doxorubicin, daunorubicin, epirubicin, mixoantrone, or idarubicin), and chromomycins (e.g., dactinomycin or plicamycin). Antimetabolites can include, for example, folic acid antagonists (e.g., methotrexate), pyrimidine antagonists (e.g., 5-flurouracil, foxuridine, cytarabine, capecitabine, or gemcitabine), purine antagonists (e.g., 6-mercaptopurine or 6-thioguanine), and adenosine deaminase inhibitors (e.g., cladribine, fludarabine, nelarabine, or pentostatin). Topoisomerase inhibitors can include, for example, topoisomerase I inhibitors (e.g., irinotecan or topotecan) and topoisomerase II inhibitors (e.g., amsacrine, etoposide, etoposide phosphate, or teniposide). Anti-neoplastics can include, for example, ribonucleotide reductase inhibitors (e.g., hydroxyurea), adrenocortical steroid inhibitors (e.g., mitotane), enzymes (e.g., asparaginase or pegaspargase), antimicrotubule agents (e.g., estramustine), and retinoids (e.g., bexarotene, isotretinoin, or tretinoin).

The drug component can also be an anthracycline, a camptothecin, a tubulin inhibitor, a maytansinoid, a calicheamycin, a pyrrolobenzodiazepine dimer (PBD), an auristatin, a nitrogen mustard, an ethylenimine derivative, an alkyl sulfonate, a nitrosourea, a triazene, a folic acid analog, a taxane, a COX-2 inhibitor, a pyrimidine analog, a purine analog, an antibiotic, an enzyme inhibitor, an epipodophyllotoxin, a platinum coordination complex, a vnca alkaloid, a substituted urea, a methyl hydrazine derivative, an adrenocortical suppressant, a hormone antagonist, an antimetabolite, an alkylating agent, an antimitotic, an anti-angiogenic agent, a tyrosine kinase inhibitor, an mTOR inhibitor, a heat shock protein (HSP90) inhibitor, a proteosome inhibitor, an HDAC inhibitor, a pro-apoptotic agent, and a combination thereof.

Specific drugs of use may be selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A 13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839. Preferably, the drug is SN-38.

In an aspect the combination therapy is a protein conjugate. The protein conjugate can carry a payload that can be a therapeutic, diagnostic, or a reporter. A single molecule of the therapeutic, diagnostic or reporter may be present or two or more molecules may be present. The therapeutic can be a chemotherapeutic including, for example, any of those described herein such as a radionucleotide, an alkylating agent, a plant alkaloid, an antitumor antibiotic, an antimetabolite, a topoisomerase inhibitor, and/or an anti-neoplastic. The payload of the conjugate can be any one or more of these therapeutics, diagnostics and/or reporters. The protein can be a fragment, a monomer, a dimer, or a multimeric protein. The protein can be an antibody, an antibody fragment or derivative, a single chain antibody, an enzyme, cytokine, chemokine, receptor, blood factor, peptide hormone, toxin, and/or transcription factor.

Many conjugating reagents can be used to conjugate a payload to a protein. Such reagents may contain at least one functional group capable of reacting with a protein or peptide. For example, the conjugating reagent may comprise a functional group capable of reacting with at least one electrophile or, especially, nucleophile, present in the protein, the functional group being attached to the payload via the linker. Any type of known conjugation reaction may be used to form the conjugate. For example, the reaction can be carried out using the known methods of thiol bonding, amine conjugation, or click chemistry. The reagent may contain a maleimide group, an N-hydroxysuccinimide group, a click-chemistry group, for example an azide or alkyne group, an amine group, a carboxyl group, a carbonyl group, or an active ester group. Other possible approaches include the use of proteins that have been recombinantly engineered with an amino acid specifically for conjugation such as engineered cysteines or non-natural amino acids, and enzymatic conjugation through a specific enzymatic reaction such as with transglutaminase. The reaction site on the protein may be either nucleophilic or electrophilic in nature. Common protein conjugation sites are at lysine or cysteine amino acid residues or carbohydrate moieties. Alternatively, conjugation may occur at a polyhistidine tag which has been attached to a binding protein.

A conjugating reagent can be advantageously capable of reacting with a nucleophile in a protein and hence becoming chemically bonded thereto. In these examples, the conjugating reagent typically includes at least one leaving group which is lost on reaction with a nucleophile. The conjugating reagent may, for example, include two or more leaving groups. The conjugating reagent can be capable of reacting with two nucleophiles. The conjugating reagent can comprise at least two leaving groups. When two or more leaving groups are present, these may be the same or different. Alternatively, a conjugating reagent may contain a single group which is chemically equivalent to two leaving groups and which single group is capable of reacting with two nucleophiles. Nucleophilic groups include, for example, sulfur atoms and amine groups, and nucleophilic groups in proteins are for example provided by cysteine, lysine or histidine residues. Nucleophilic groups can be a sulfur atom present in a cysteine residue of a protein. Such structures may be obtained by reduction of a disulfide bond in the protein. The nucleophilic group may be an imidazole group in a histidine residue of the protein, e.g., as present in a polyhistidine tag.

The conjugates can contain a linker which connects the therapeutic, diagnostic or labelling agent to the protein or peptide in the conjugate. The backbone of the linker can be a continuous chain of atoms which runs from the therapeutic, diagnostic or labelling agent at one end to the protein or peptide at the other end. The linker may contain a degradable group, i.e. it may contain a group which breaks under physiological conditions, separating the payload from the protein to which it is, or will be, bonded. Alternatively, the linker is not cleavable under physiological conditions. Where a linker breaks under physiological conditions, it is preferably cleavable under intracellular conditions. Where the target is intracellular, preferably the linker is substantially insensitive to extracellular conditions (i.e. so that delivery to the intracellular target of a sufficient dose of the therapeutic agent is not prohibited).

Where the linker contains a degradable group, this is generally sensitive to hydrolytic conditions, for example it may be a group which degrades at certain pH values (e.g. acidic conditions). Hydrolytic/acidic conditions may for example be found in endosomes or lysosomes. Examples of groups susceptible to hydrolysis under acidic conditions include hydrazones, semicarbazones, thiosemicarbazones, cis-aconitic amides, orthoesters and ketals. The degradable linker can also be an acid-cleavable linker or a reducible linker. The reducible linker may comprise a disulfide group. The linker may also contain a group which is susceptible to enzymatic degradation, for example it may be susceptible to cleavage by a protease (e.g. a lysosomal or endosomal protease) or peptidase. For example, it may contain a peptidyl group comprising at least one, for example at least two, or at least three amino acid residues (e.g. Phe-Leu, Gly-Phe-Leu-Gly, Val-Ala, Val-Cit, Phe-Lys, Glu-Glu-Glu). For example, it may include an amino acid chain having from 1 to 5, for example 2 to 4, amino acids. The enzyme cleavable linker can also comprise a chemical group which can be cleaved or degraded by one or more lysosomal enzymes. Suitable groups include, for example, a valine-citrulline dipeptide group, a phenylalanine-lysine dipeptide group, and a β-glucuronide group.

When the protein in the protein conjugate is an antibody (e.g., full length, fragment, and/or single chain) one end of the first linker can be covalently attached to the antibody. The antibody-reactive end of the linker can be a site that is capable of conjugation to the antibody through a cysteine thiol or lysine amine group on the antibody, and so can be a thiol-reactive group such as a double bond (as in maleimide) or a leaving group such as a chloro, bromo, or iodo, or an R-sulfanyl group, or an amine-reactive group such as a carboxyl group.

The CAR therapy (e.g., with a GOLD-controlled transgene) and the other therapy can be provided to a subject at the same time, or one can be provided to the subject before the other, or the CAR therapy and the other therapy can be provided in alternating cycles, or the CAR therapy together with the other therapy can be provided in cycles, or other combinations of administration can be used. The CAR therapy can be combined with an antibody conjugate (ADC) therapy where the CAR and the ADC bind to the same antigen or bind to different antigens. When the CAR and ADC bind to the same antigen, the CAR and the ADC can bind to the same or different epitopes on the antigen. One of the ADC or the CAR therapy can be provided to the subject first, and followed by the other after a period of treatment with the first. The ADC, either alone or in combination with another approved therapy (e.g. chemotherapy and/or immune checkpoint inhibitors) can be provided to the subject first to reduce the tumor burden in the subject prior to the administration of the CAR therapy. Alternatively, the ADC and CAR therapy can be provided to the subject at the same time. Or the CAR therapy can be provided first followed by the ADC therapy.

### Notch Inhibitors and Modulators

Notch can be an immunological checkpoint in T-cells. The methods and compositions for modifying Notch related suppression of T-cells can be combined with other checkpoint inhibitors and/or other immune-therapies to treat many solid tumors. In a context dependent manner, Notch signals can promote or suppress cell proliferation, cell death, acquisition of specific cell fates, or activation of differentiation programs.

T-cells express Notch receptors, and naïve CD4+ and CD8+ T-cells can express the Notch 1 and Notch 2 receptors. DLL1, DLL4, Jagged1 and Jagged2 can be ligands for inducing Notch signaling, when these ligands are immobilized (e.g., membrane bound in a cancer cell). When an immobilized ligand binds to a Notch receptor, this can induce a conformational change that allows an ADAM protease (e.g., ADAM17) to cleave the Notch receptor. Gamma secretase can then cleave the truncated receptor releasing a fragment (NICD) in the cytoplasm that can translocate to the nucleus to modulate transcription.

Binding of ligand to Notch receptors on T-cells has been shown to inhibit activation and proliferation of the T-cells. Notch receptor can be phosphorylated at multiple sites, and phosphorylation can play a role in signal transduction. Notch receptor signaling in T-cells can suppress activation and proliferation of naïve T-cells. The phosphorylation of GSK-3 and AKT are inhibited with rapid kinetics after Notch receptors engage ligand. GSK-3 is a component of Notch signaling and its phosphorylation is dependent on AKT. AKT is also essential to T-cell activation, proliferation and cytokine production.

Notch receptor activity can be inhibited by a number of different classes of agents including, for example, receptor antagonists, dominant negative receptor mutants, ADAM17 protease inhibitors, and gamma secretase inhibitors. Notch antagonists can include, for example, soluble Notch ligands such as soluble, high affinity DLL4 (Luca et al, 2015 Science 347:847-53, which is incorporated by reference in its entirety for all purposes), soluble, high affinity Jagged1, antibodies for Notch receptor (Wu et al, 2010, Nature 464:1052-57; Tran et al., 2013, J. Clin. Invest. 123:1590-1604, both of which are incorporated by reference in their entirety for all purposes), and antibodies for Notch receptor made by Merck or Oncomed.

Dominant negative mutants include, for example, PSEN1 (a component of the gamma secretase complex) mutants disclosed in Zhou et al., 2017, Proc. Natl Acad Sci 114:12731-36, which is incorporated by reference in its entirety for all purposes. Other dominant negative mutants include, for example, the PSEN1 mutant D257A/D385A and other mutants disclosed in Zhou et al, 2017, ADAM17ΔMP (e.g., Peng et al, 2010, Immunology 130:83-91, which is incorporated by reference in its entirety for all purposes), ADAM10ΔMP (Bozkulak et al, 2009, Molecular and Cellular Biology 29:5679-95, which is incorporated by reference in its entirety for all purposes).

ADAM 17 protease inhibitors include, for example, TAPI-2 (Santa Cruz Biotechnology Catalog #sc-205851), 1-Propyl-1H-imidazole (Santa Cruz Biotechnology Catalog #sc-471932), Secalciferol (Santa Cruz Biotechnology Catalog #sc-473270), Secophenol (Santa Cruz Biotechnology Catalog #sc-473288), (7R,8S,9R,10S)-rel-7,8,9,10-Tetrahydrobenzo[a]pyrene-7,8,9,10-tetrol (Santa Cruz Biotechnology Catalog #sc-474274), Boc-L-glutamic acid gamma-benzyl ester 4-oxymethylphenylacetamidomethyl resin (Santa Cruz Biotechnology Catalog #sc-476580), 3-[(4-Methyl-1-piperazinyllimino)methyl] rifamycin 0 (Santa Cruz Biotechnology Catalog #sc-487922), GI 254023X (Santa Cruz Biotechnology Catalog #sc-490114), (2Z)-6-Chloro-2-[(2,4-dimethoxyphenyl)imino]-N-(tetrahydrofuran-2-ylmethyl)-2H-chromene-3-carboxamide (Santa Cruz Biotechnology Catalog #sc-491865), {[4-(2-Oxo-2H-chromen-3-yl)-1,3-thiazol-2-yl]thio}acetic acid (Santa Cruz Biotechnology Catalog #sc-493593), 4-(Dimethylamino)-N-{1-[3-(2-thienyl)-1H-pyrazol-5-yl]piperidin-4-yl}benzamide (Santa Cruz Biotechnology Catalog #sc-495081), 2-{[(2,5-Diethoxyphenyl)amino]methyl}-6-ethoxyphenol (Santa Cruz Biotechnology Catalog #sc-495315), Etozolin-d3 Hydrochloride (Santa Cruz Biotechnology Catalog #sc-497422), Erythrolosamine (Santa Cruz Biotechnology Catalog #sc-498341), Desmethyl doxylamine-d5 (Santa Cruz Biotechnology Catalog #sc-500285), N-Demethyl N-acetyl alogliptin-2,2,2-trifluoroacetate (Santa Cruz Biotechnology Catalog #sc-500411), GI 254023X (Tocris Catalog #3995), TAPI 0 (Tocris Catalog #5523), TAPI-1 (Tocris Catalog #6162), TAPI-2 (Tocris Catalog #6013), and TMI-1 (Tocris Catalog #5960).

Gamma secretase inhibitors fall into a number of classes and subclasses: peptide isosteres (e.g., aspartyl proteinase transition-state analogs) and small molecules (e.g., azepines, sulfonamides). Many gamma secretase inhibitors are commercially available including, for example, DAPT (GSI-IX) (Selleckchem Catalog #S2215), R04929097 (Selleckchem Catalog #S1575), Semagacestat (LY450139) (Selleckchem Catalog #S1594), MK-0752 (Selleckchem Catalog #S2660), Avagacestat (BMS-708163) (Selleckchem Catalog #S1262), MDL-28170 (Selleckchem Catalog #S7394), Debenzazepine (Selleckchem Catalog #S2711), LY411575 (Selleckchem Catalog #S2714), Nirogacestat (Selleckchem Catalog #S8018), L-685,458 (Selleckchem Catalog #S7673), FPS-ZM1 (Selleckchem Catalog #S8185), Crenigacestat (Selleckchem Catalog #S7169), CHF-5074 (Selleckchem Catalog #S7323), NGP-555 (Selleckchem Catalog #S8603).

Recombinant Notch antagonists (e.g., antibodies or soluble ligands) and/or dominant negative mutants can be delivered as a payload(s) by the eukaryotic cell (e.g., T-cell, NK cell, CAR NK cell, or CAR T-cell). The Notch antagonists (e.g., antibodies or soluble ligands) and/or dominant negative mutants can be constitutively expressed or can be inducibly expressed. Inducible expression can involve an inducible promoter and/or inducible post-transcriptional control such as, for example, an RDE, an RNA control device, or a degron.

The payload can be placed under the control of an RDE so that it is expressed upon activation of the eukaryotic cell (e.g., T-cell or NK cell). Some dominant negative mutants can be placed under the control of an RDE and when the T-cell is expanded with CD3/CD28 the T-cell expresses the dominant negative mutant prior administration to a subject.

An antibody or soluble ligand payload can be constitutively expressed or when under inducible expression, expression can be induced at a desired time. For example, expression can be induced prior to when the eukaryotic cell (e.g., T-cell of NK cell) reaches the target site (e.g., a tumor). Expression can also be induced when or after the eukaryotic cell (e.g., T-cell of NK cell) reaches the target site (e.g., a tumor). If expression has been placed under ligand inducible control, ligand can be added to the eukaryotic cell at the desired time.

Small molecule Notch inhibitors can be administered to a subject systemically (e.g., orally or via injection), or locally (e.g., intratumor). Many of the protease inhibitors have been formulated for oral administration and subjects can be dosed orally at an appropriate time to produce a desired amount of small molecule at the target site based on the known PK properties of the small molecule.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1. Control of T-Cell Effector Activity with an RDE-CAR

A RDE Car is made using the third generation anti-CD19 CAR cassette described in WO 2012/079000, which is hereby incorporated-by-reference in its entirety for all purposes), and the 3′-UTR of the gene encoding IL-2 (NCBI Reference Sequence Number: NM_000586.3), which is hereby incorporated by reference in its entirety for all purposes). A nucleic acid encoding the IL-2 3′-UTR is engineered into the anti-CD19 CAR cassette in an appropriate expression vector. The IL-2, 3′-UTR sequence used was:

(SEQ ID NO: 28)
taattaagtgcttcccacttaaaacatatcaggccttctATTTATTTAaa
tATTTAaattttatATTTAttgttgaatgtatggtttgctacctattgta
actattattcttaatcttaaaactataaatatggatcttttatgattctt
tttgtaagccctaggggctctaaaatggtttcacttATTTAtcccaaaat
ATTTAttattatgttgaatgttaaatatagtatctatgtagattggttag
taaaactATTTAataaatttgataaatataaa

The anti-CD19 RDE CAR and anti-CD19 CAR constructs are transfected by routine methods into different populations of T-cells (primary human T-cells), and stable populations of T-cells are selected using appropriate antibiotics (or other selection schemes). T-cell populations with anti-CD19 RDE CARs (CD19⁻/CD22⁻/CD3⁺) and T-cell populations with anti-CD19 CARs (CD19⁻/CD22⁻/CD3⁺) are activated by co-incubation with anti-CD3/CD28 beads and allowed to return to quiescent state after debeading.

Quiescent anti-CD19 RDE CAR T-cells are co-cultured with CD19⁺/CD22⁺/CD3⁻ Raji target cells at RDE CAR T-cell:Raji target ratios of 2:1, 5:1, and 10:1. The glycolysis activator glucose is added to the culture medium at concentrations in the range of 1.0 mM to 10 mM (1 mM, 2 mM, 3 mM, 4 mM, 5 mM, 7.5 mM and 10 mM). The RDE-CAR T-cells and the Raji cells are grown together for 24 hours. Cultures are washed, and then stained with anti-CD22 and anti-CD3 reagents, followed by counting of CD22⁺ (Raji target cells) and CD3⁺ cells (CAR T-cells). These measurements will identify the target cell killing rate (e.g., half-life) and the proliferation rate of the RDE-CAR T-cells at different levels of RDE-CAR expression.

Activated anti-CD19 RDE CAR T-cells are co-cultured with CD19⁺/CD22⁺/CD3⁻ Raji target cells at RDE CAR T-cell:Raji target ratios of 2:1, 5:1, and 10:1. The glycolysis activator glucose is added to the culture medium at concentrations in the range of 1.0 mM to 10 mM (1 mM, 2, mM, 3 mM, 4 mM, 5 mM, 7.5 mM and 10 mM). The RDE-CAR T-cells and the Raji cells are grown together for 24 hours. Samples from culture media are taken and tested for IL-2 by ELISA.

As a control activated anti-CD19 CAR T-cells are co-cultured with CD19⁺/CD22⁺/CD3⁻ Raji target cells at CAR T-cell:Raji target ratios of 2:1, 5:1, and 10:1. The glycolysis activator glucose is added to the culture medium at concentrations in the range of 1.0 mM to 10 mM (1 mM, 2 mM, 3 mM, 4 mM, 5 mM, 7.5 mM and 10 mM). The CAR T-cells and the Raji cells are grown together for 24 hours. Cultures are washed, and then stained with anti-CD22 and anti-CD3 reagents, followed by counting of CD22⁺ (Raji target cells) and CD3⁺ cells (CAR T-cells).

As a control, activated anti-CD19 CAR T-cells are co-cultured with CD19⁺/CD22⁺/CD3⁻ Raji target cells at CAR T-cell:Raji target ratios of 2:1, 5:1, and 10:1. The glycolysis activator glucose is added to the culture medium at concentrations in the range of 1.0 mM to 10 mM (1 mM, 2 mM, 3 mM, 4 mM, 5 mM, 7.5 mM and 10 mM). The CAR T-cells and the Raji cells are grown together for 48 hours. Samples from culture media are taken and tested for IL-2 by ELISA.

### Example 2: Removal of MicroRNA Binding Sites from an RDE

The AU-rich element from the 3′-UTR of IL-2 has mir-181 and mir 186 microRNA binding sites. Different combinations of the microRNA sites were removed from the 3′-UTR of IL-2. When the MIR186 micro-RNA sites were removed from the 3′-UTR of IL-2 the dynamic range of expression from constructs with this UTR increased 50 fold. The modified IL-2, 3′-UTR replaces CTT in the sequence with GAA and is shown below (the new GAA is underlined in the sequence):

(SEQ ID NO: 29)
taattaagtgcttcccacttaaaacatatcaggccttctATTTATTTAaa
tATTTAaattttatATTTAttgttgaatgtatggtttgctacctattgta
actattattcttaatcttaaaactataaatatggatcttttatgattGAA
tttgtaagccctaggggctctaaaatggtttcacttATTTAtcccaaaat
ATTTAttattatgttgaatgttaaatatagtatctatgtagattggttag
taaaactATTTAataaatttgataaatataaa

The AU-rich element from the 3′UTR of IFNg also has micro-RNA binding sites characterized as mir-125. The sequence of the IFNg RDE is:

(SEQ ID NO: 30)
tggttgtcctgcctgcaatatttgaattttaaatctaaatctATTTAtta
atATTTAacattATTTAtatggggaatatatttttagactcatcaatcaa
ataagtATTTAtaatagcaacttttgtgtaatgaaaatgaatatctatta
atatatgtattATTTAtaattcctatatcctgtgactgtctcacttaatc
ctttgttttctgactaattaggcaaggctatgtgattacaaggctttatc
tcaggggccaactaggcagccaacctaagcaagatcccatgggttgtgtg
tttatttcacttgatgatacaatgaacacttataagtgaagtgatactat
ccagttactgccggtttgaaaatatgcctgcaatctgagccagtgcttta
atggcatgtcagacagaacttgaatgtgtcaggtgaccctgatgaaaaca
tagcatctcaggagatttcatgcctggtgcttccaaatattgttgacaac
tgtgactgtacccaaatggaaagtaactcatttgttaaaattatcaatat
ctaatatatatgaataaagtgtaagttcacaacta

Different combinations of the micro-RNA sites were removed from the 3′UTR of IFNg and tested for increased expression. When the mir125 micro-RNA sites were removed from the 3′-UTR of IFN-γ the expression rate from constructs with this UTR is increased.

Expression of GFP in T-cells, transfected with the RDE-GFP plus the microRNA sites, is compared to expression of GFP in T-cells with the RDE-GFP in which the microRNA sites have been removed, following activation with CD3/CD28 beads for 24 hours. The removal of the microRNA sites increased expression of the GFP by a factor of between 2-5 after 24 hours, relative to the cells with microRNA sites.

### Example 3: Payload Delivery to DLBCL Using an Anti-CD19 CAR T-Cell

Anti-CD19 CAR T-lymphocytes are used in this example. These CAR T-lymphocytes are further engineered to include a construct encoding a PD-1 inhibitor under the control of the 3′-UTR of IL2 that has been modified by removal of the MIR186 sites. PD-1 inhibitors expressed by the construct include, for example, Pembrolizumab (Keytruda®), Nivolumab (Opdivo®), Cemiplimab (Libtayo®), Atezolizumab (Tecentriq®), Avelumab (Bavencio®), Durvalumab (Imfinzi®), BMS-936558, Lambrolizumab, or polypeptides derived from these drugs. Other PD-1 inhibitors that may be expressed by the construct include those disclosed in Herbst et al., J Clin Oncol., 31:3000 (2013); Heery et al., J Clin Oncol., 32:5s, 3064 (2014); Powles et al., J Clin Oncol, 32:5s, 5011(2014); Segal et al., J Clin Oncol., 32:5s, 3002 (2014), or U.S. Pat. Nos. 8,735,553; 8,617,546; 8,008,449; 8,741,295; 8,552,154; 8,354,509; 8,779,105; 7,563,869; 8,287,856; 8,927,697; 8,088,905; 7,595,048; 8,168,179; 6,808,710; 7,943,743; 8,246,955; and 8,217,149.

T-cell populations with anti-CD19 CARs/PD-1 (CD19−/CD22−/CD3+) are activated by co-incubation with anti-CD3/CD28 beads. T-cells with anti-CD19 CARs/PD-1 inhibitor were incubated with theophylline at 0, 75 and 250 μM for 72 hours. Activated anti-CD19 CAR/PD-1 T-cells were co-cultured with CD19+/CD22+/CD3− Raji target cells at CAR/PD-1 T-cell:Raji target ratios of 2:1, 5:1, and 10:1. Ligand for the RNA control device, theophylline is maintained in the culture medium at concentrations of 0 μM, 75 μM, and 250 μM. The CAR/PD-1 T-cells and the Raji cells are grown together for 18 hours. Cultures are washed, and then stained with anti-CD22 and anti-CD3 reagents, followed by counting of CD22+ (Raji target cells) and CD3+ cells (CAR T-cells). Samples from culture media are also taken at 6, 12 and 18 hours, and tested for PD-1 inhibitor by ELISA.

### Example 4: Payload Delivery to AML Using an Anti-CD133 CAR T-Cell

A CAR is made using the anti-CD20 CAR cassette described in Budde 2013 (Budde et al. PLoS1, 2013 doi:10.1371/journal.pone.0082742, which is hereby incorporated-by-reference in its entirety for all purposes), with the anti-CD133 mAb 293C3-SDIE is used for the extracellular element (Rothfelder et al., 2015, ash.confex.conasb/2015/webprogramn/Pape81121.html, which is incorporated by reference in its entirety for all purposes) replacing the anti-CD20 extracellular domain. A nucleic acid encoding the anti-CD20 CAR cassette is engineered to replace the anti-CD20 extracellular domain with the anti-CD133 element. The anti-CD133 CAR is cloned into appropriate expression vectors.

These anti-CD133 CAR constructs are transfected by routine methods into T-lymphocytes (Jurkat cells and/or primary human T-lymphocytes), and stable populations of T-lymphocytes are selected using appropriate antibiotics (or other selection schemes).

These CAR T-lymphocytes are further engineered to include a construct encoding a PD-1 inhibitor under the control of the RDE from the 3′-UTR of IL2 that has been modified by removal of a MIR186 site. PD-1 inhibitors expressed by the construct include, for example, Pembrolizumab (Keytruda®), Nivolumab (Opdivo®), Cemiplimab (Libtayo®), Atezolizumab (Tecentriq®), Avelumab (Bavencio®), Durvalumab (Imfinzi®), BMS-936558, Lambrolizumab, or polypeptides derived from these drugs. Other PD-1 inhibitors that may be expressed by the construct include those disclosed in Herbst et al., J Clin Oncol., 31:3000 (2013); Heery et al., J Clin Oncol., 32:5s, 3064 (2014); Powles et al., J Clin Oncol, 32:5s, 5011(2014); Segal et al., J Clin Oncol., 32:5s, 3002 (2014), or U.S. Pat. Nos. 8,735,553; 8,617,546; 8,008,449; 8,741,295; 8,552,154; 8,354,509; 8,779,105; 7,563,869; 8,287,856; 8,927,697; 8,088,905; 7,595,048; 8,168,179; 6,808,710; 7,943,743; 8,246,955; and 8,217,149.

T-lymphocyte populations with anti-CD133 CAR/PD-1 inhibitor are activated by co-incubation with anti-CD3/CD28 beads.

Activated anti-CD133 CAR/PD-1 inhibitor T-lymphocytes are co-cultured with CD133⁺/CD3⁻ AML target cells (e.g., U937, MV4-11, MOLM-14, HL-60 and/or KGla) at anti-CD133 CAR:AML target ratios of 2:1, 5:1, and 10:1. The anti-CD133 CAR/PD-1 inhibitor T-lymphocytes and the AML cells are grown together for 48 hours. Cultures are washed, and then stained with anti-CD133 and anti-CD3 reagents, followed by counting of CD133Y (AML target cells) and CD3+ cells (anti-CD133 CAR). These measurements will identify the target cell killing rate (e.g., half-life) and the proliferation rate of the anti-CD133 CAR/PD-1 inhibitor T-lymphocytes at different levels of CAR expression. Samples from culture media are also taken at 12, 24, 26 and 48 hours, and tested for PD-1 inhibitor by ELISA.

### Example 5: An RDE Construct for Expressing a Second Transgene

Constructs were made using an anti-CD19 CAR cassette as described in WO 2012/079000, which is hereby incorporated-by-reference in its entirety for all purposes), and a GFP-RDE1 (3′-UTR from IFNg) insert. These two inserts/cassettes were placed in the same lenti virus construct. The anti-CD19 CAR cassette and the insert with the GFP-RDE are transcribed in opposite directions, and the control regions for each are located in between the two insert/cassettes. The control region for the GFP-RDE insert was MinP and the RDE was the endogenous 3′-UTR of IFNg. The control region of the anti-CD19 CAR cassette was MND (as described above). CD4+ T-cells were transduced with the bicistronic construct.

The transduced T cells were allowed to return to resting state, and then were tested after stimulation as follows. For the ‘no stimulation’ set, transduced T-cells were incubated for 24 h alone in medium. For the ‘Raji co-culture’ set and the “CD3/CD28 Beads” set, CD19+ Raji B cells or anti-CD3/anti-CD28 beads were incubated with the transduced T cells for 24 h. At 24 h, the T cells were stained for CD25 and CD69, which are activation markers, and subject to flow cytometry to measure these markers and GFP expression in the T cells.

The transduced T-cells showed an increase in fluorescence when cultured with Raji target cells (activate CAR) of 1.0% to 6.5% (about 6.5 fold), and increase in fluorescence when cultured with CD3/CD28 beads (activate TCR) of 1.0% to 4.4% (about 4.4 fold). The transformed T-cells showed a change in activated cells in the population when cultured with Raji cells of 0.9% to 84.8%, and when cultured with CD3/CD28 beads of 0.9% to 90.8%.

### Example 6: A Modified RDE2 Construct for Expressing a Second Transgene

Constructs were made using an anti-CD19 CAR cassette as described in Examples 11 and 12, and a GFP-RDE2.1 (IL-2 RDE) insert. The RDE2.1 was modified to remove the MIR186 microRNA sites, altering nucleotides from the 3′-UTR of IL-2 which was used as RDE2.

These two inserts/cassettes were placed in the same lenti virus construct. The anti-CD19 CAR cassette and the insert with the GFP-RDE are transcribed in opposite directions, and the control regions for each are located in between the two insert/cassettes. The control region for the GFP-RDE insert was a MinP. The control region of the anti-CD19 CAR cassette in was MND (as described above). CD4+ T-cells were transduced with the bicistronic construct.

The transduced T cells were allowed to return to resting state, and then were tested after stimulation as follows. For the ‘no stimulation’ set, transduced T-cells were incubated for 24 h alone in medium. For the ‘Raji co-culture’ set and the “CD3/CD28 Beads” set, CD19+ Raji B cells or anti-CD3/anti-CD28 beads were incubated with the transduced T cells for 24 h. At 24 h, the T cells were stained for CD25 and CD69, which are activation markers, and subject to flow cytometry to measure these markers and GFP expression in the T cells.

The transduced T-cells showed a change in activated cells in the population when cultured with Raji cells of 3.9% to 12.1%, and when cultured with CD3/CD28 beads of 3.9% to 11.1%.

### Example 7: An RDE Construct for Expressing a Luciferase Transgene

Constructs were made using an anti-CD19 CAR cassette as described in WO 2012/079000, which is hereby incorporated-by-reference in its entirety for all purposes), and a Luciferase-RDE1 (3′-UTR of IFNg, Gold1) insert or a Luciferase-3′-UTR (a 3′-UTR that does not confer differential transgene translation in response to metabolic state of the cell, 3′-UTR). The anti-CD19 CAR cassette and the insert with the luciferase-RDE1 are transcribed in opposite directions, and the control regions for each are located in between the two insert/cassettes. The control region for the Luciferase-RDE1 insert and Luciferase-3′-UTR were either a MinP promoter or an NFAT promoter having the sequences of:

(MinP)
SEQ ID NO: 31
TAGAGGGTATATAATGGAAGCTCGACTTCCAG
(NFAT)
SEQ ID NO: 32
GGAGGAAAAACTGTTTCATACAGAAGGCGTGGAGGAAAAACTGTTTCAT
ACAGAAGGCGTGGAGGAAAAACTGTTTCATACAGAAGGCGTAGATCTAG
ACTCTAGAGGGTATATAATGGAAGCTCGAATTC

The control region of the anti-CD19 CAR cassette was the MND promoter. CD4+ T-cells were transduced with the bicistronic construct.

The transduced T cells were allowed to return to resting state, and then were tested after stimulation as follows. For the ‘no stimulation’ set, transduced T-cells were incubated for 24 h alone in medium. For the ‘Raji co-culture’ set and the “CD3/CD28 Beads” set, CD19+ Raji B cells or anti-CD3/anti-CD28 beads were incubated with the transduced T cells for 24 h. At 24 h, the T cells were stained for CD25 and CD69, which are activation markers, and subject to flow cytometry to measure these markers and luciferase expression in the T cells.

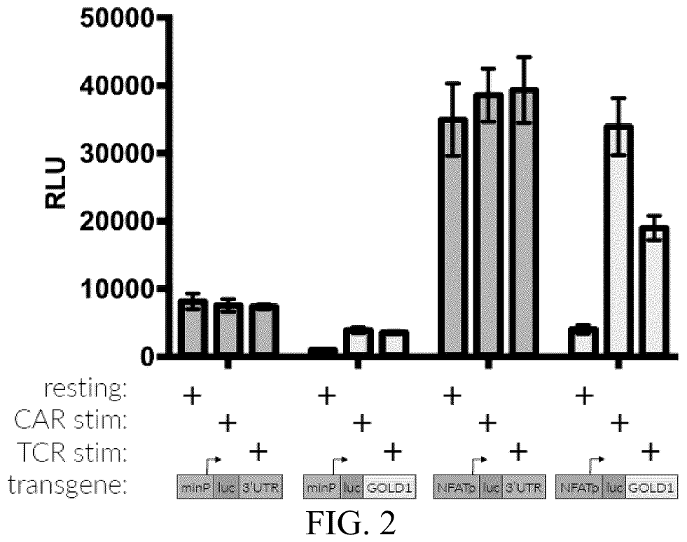

FIG. 2 shows that the transduced T-cells had an increase in bioluminescence when cultured with Raji target cells (activate CAR) or when cultured with CD3/CD28 beads (activate TCR) as compared to bioluminescence of T-cells at resting. The T-cells with a NFAT promoter and the 3′-UTR of IFNg (Gold1) showed a larger on-off response from CAR stimulation versus TCR stimulation. Under all conditions, T-cells with Gold1 had lower amounts of bioluminescence than T-cells under the same conditions (and same promoter) with Luciferase that was not controlled by the 3′UTR of IFNg (3′-UTR).

### Example 8: Comparison of RDEs Controlling Luciferase

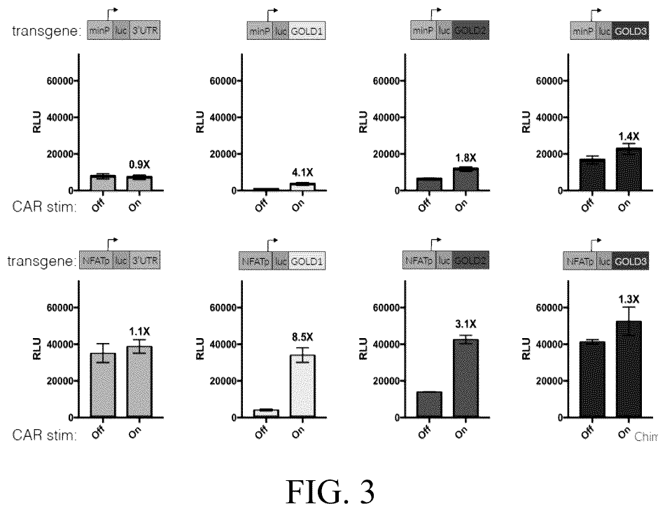

Constructs were made using an anti-CD19 CAR cassette as described in WO 2012/079000, which is hereby incorporated-by-reference in its entirety for all purposes), and a Luciferase-RDE1 (3′ UTR of IFNg, Gold1) insert, a Luciferase-RDE2 (3′-UTR of IL-2, Gold2) insert, a Luciferase-RDE3 (3′-UTR of IL-2 modified as described above to remove the mir186 sites, Gold3), or a Luciferase-3′-UTR (a 3′-UTR that does not confer differential transgene translation in response to metabolic state of the cell, 3′-UTR). Combinations of these inserts/cassettes shown in FIG. 3 were placed in the similar lenti virus constructs. The anti-CD19 CAR cassette and the insert with the luciferase-RDE are transcribed in opposite directions, and the control regions for each are located in between the two insert/cassettes. The control region for the Luciferase-RDE insert and Luciferase-3′-UTR were either a MinP promoter or an NFAT promoter. The control region of the anti-CD19 CAR cassette was the MND promoter, and CD4+ T-cells were transduced with the bicistronic construct.

The transduced T cells were allowed to return to resting state, and then were tested after stimulation as follows. For the ‘no stimulation’ set, transduced T-cells were incubated for 24 h alone in medium. For the ‘Raji co-culture’ set CD19+ Raji B cells were incubated with the transduced T cells for 24 h. At 24 h, the T cells were stained for CD25 and CD69, which are activation markers, and subject to flow cytometry to measure these markers and luciferase expression in the T cells.

FIG. 3 shows that the transduced T-cells had an increase in bioluminescence when cultured with Raji target cells (activate CAR) as compared to bioluminescence of T-cells at resting for constructs with RDE1 (Gold1), RDE2 (Gold2), or RDE3 (Gold3). The T-cells with a NFAT promoter and the RDE1 showed a larger on-off response than T-cells with a MinP promoter and the corresponding RDE. Under all conditions, T-cells with an RDE controlling luciferase had lower amounts of bioluminescence than T-cells with luciferase cassettes that were not controlled by an RDE. Combined with the MinP promoter, RDE1 gave a 4.1-fold increase in bioluminescence with CAR stimulation, RDE2 gave a 1.8-fold increase in bioluminescence, and RDE3 gave a 1.4-fold increase. Combined with the NFAT promoter, RDE1 gave a 8.5-fold increase in bioluminescence with CAR stimulation, RDE2 gave a 3.1-fold increase in bioluminescence, and RDE3 gave a 1.3-fold increase. With either promoter, the RDE3 construct gave the highest amount of bioluminescence, the RDE1 construct gave the lowest amount of bioluminescence, and the RDE2 construct gave an amount of bioluminescence between RDE3 and RDE1.

### Example 9: An RDE Construct for Expressing IL-12

Constructs were made using an anti-CD19 CAR cassette as described in WO 2012/079000, which is hereby incorporated-by-reference in its entirety for all purposes), and an IL-12-RDE1 (3′-UTR of IFNg) insert or an IL-12 3′-UTR (a 3′-UTR that does not confer differential transgene translation in response to metabolic state of the cell). The anti-CD19 CAR cassette and the insert with the IL-12-RDE1 are transcribed in opposite directions, and the control regions for each are located in between the two insert/cassettes. The control region for the IL-12-RDE1 insert and IL-12 3′-UTR were either a minP promoter or an NFAT promoter. The control region of the anti-CD19 CAR cassette was the MND promoter. CD4+ T-cells were transduced with the bicistronic construct.

The transduced T cells were allowed to return to resting state, and then were tested after stimulation as follows. For the ‘no stimulation’ set, transduced T-cells were incubated for 24 h alone in medium. For the ‘Raji co-culture’ set, CD19+ Raji B cells were incubated with the transduced T cells for 24 h. At 24 h, the T cells were stained for CD25 and CD69, which are activation markers, and subject to flow cytometry to measure these markers. IL-12 expression in the T cells was measured by ELISA.

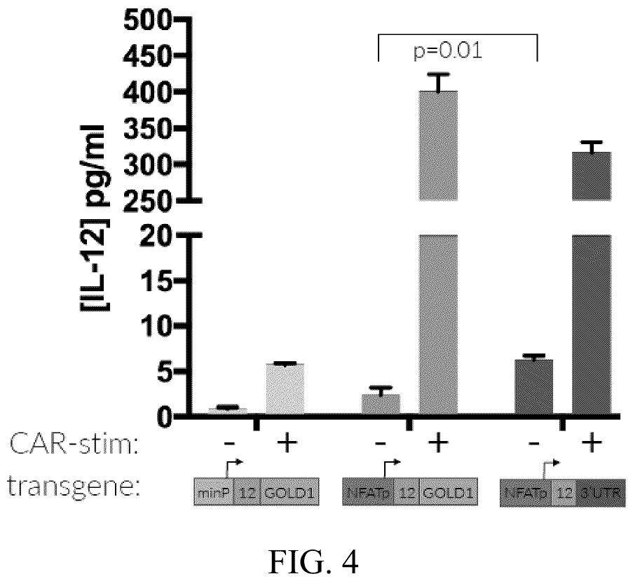

FIG. 4 shows that the transduced T-cells had an increase in IL-12 expression when cultured with Raji target cells (activate CAR) as compared to IL-12 expression of T-cells at resting using constructs controlled by the MinP promoter or NFAT promoter. T-cells with the NFAT promoter and RDE1 (Gold 1) showed a 168-fold change in IL-12 expression form resting to CAR stimulation. T-cells with the NFAT promoter and a 3′-UTR (not responsive to CAR stimulation, 3′-UTR) showed a 50-fold change in expression, and a minP promoter with RDE1 (Gold 1) showed a 6.3 fold change in expression.

### Example 10: AU Elements and Steady State Expression

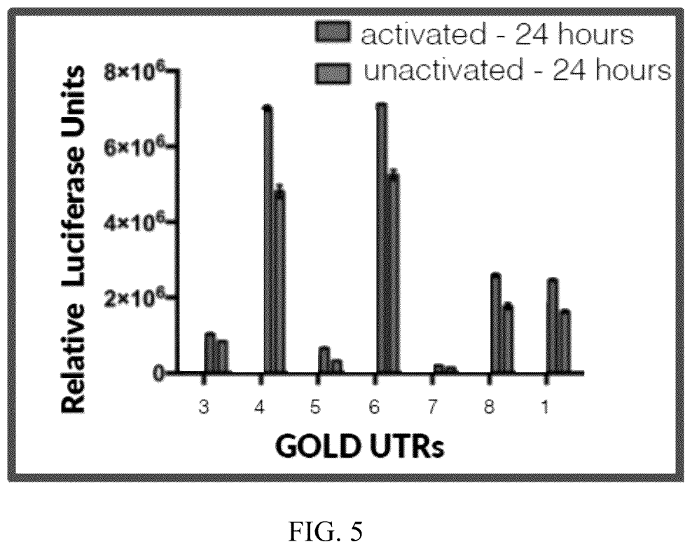

Constructs were made with different RDEs operably linked to a nucleic acid encoding luciferase. The different RDEs used were AU 4 (CTLA4), AU 13 (IL-5), AU 14 (IL-6), AU 15 (IL-9), AU 16 (IL-10), AU 17 (IL-13), and AU 101 (IFNg). These luciferase-AU constructs were transduced into primary T-cells. After the cells returned to the resting stage they were plated and sham induced (basal) or induced with anti-CD3 and anti-CD28 antibody (activated). At 24 hours post activation the amount of luciferase units in each was measured. These amounts are plotted in the bar graph of FIG. 5.

The AU elements in this example had different basal expression levels, different induced expression levels (at 24 hours), and different levels of fold induction. The AU constructs showed different amounts of basal expression, different amounts of induced expression and different amounts of fold induction (or dynamic range).

### Example 11: AU Elements and Expression Parameters

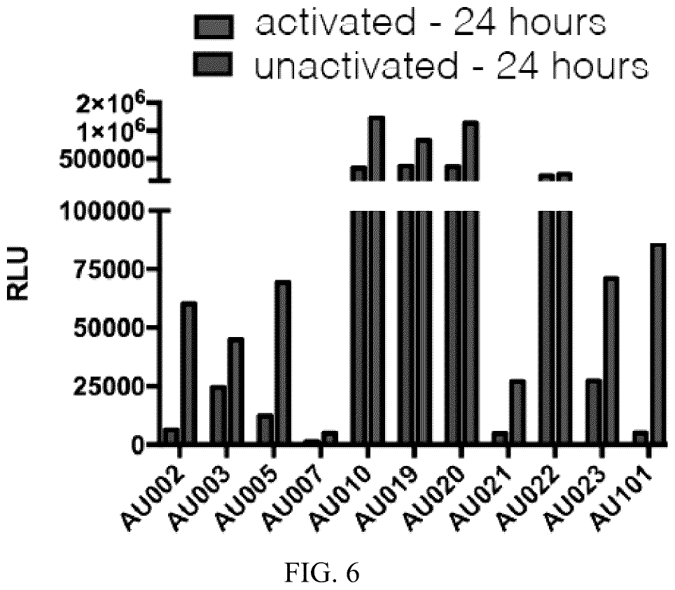

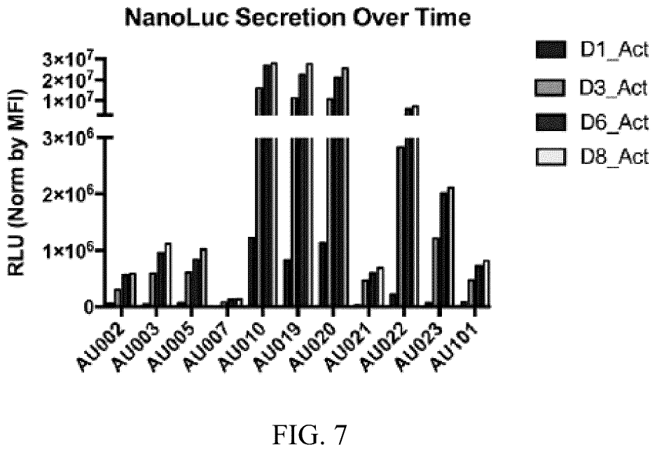

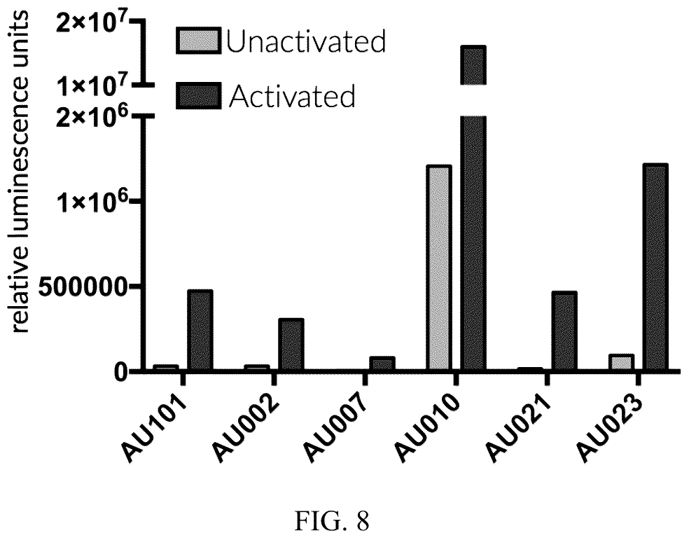

Constructs were made with different RDEs operably linked to a nucleic acid encoding luciferase. The different RDEs used were AU 2 (CSF2), AU 3 (CD247), AU 5 (EDN1), AU 7 (SLC2A1), AU 10 (Myc), AU 19 (TMEM-219), AU 20 (TMEM-219snp), AU 21 (CCR7), AU 22 (SEM-A4D), AU 23 (CDC42-SE2), and AU 101 (IFNg). These luciferase-AU constructs were transduced into primary T-cells. After the cells returned to the resting stage they were plated and either not treated (basal) or activated with anti-CD3 and anti-CD28 antibody (activated). At 24 hours post activation the amount of luciferase units in each was measured. These amounts are plotted in the bar graph of FIG. 6. Alternatively, after the cells returned to resting stage they were plated into 96-well plates in quadruplicate for measuring at each time point: 1 day, 3 days, 6 days and 8 days. The cells were either not treated or activated with anti-CD3 and anti-CD28 antibody, and luciferase activity was measured at 1 day, 3 days, 6 days and 8 days. These results are plotted in the bar graph of FIG. 7, and shown in Table 1 below. FIG. 8 shows selected data plotted in a bar graph. The numbers in parentheses in Table 1 below are the Luciferase Units on Days 3, 6, and 8 divided by the Luciferase Units of Day 1.

TABLE 1
Luciferase Units

| AU Construct | Day 1 | Day 3 | Day 6 | Day 8 |
|---|---|---|---|---|
| AU 2 (CSF2) | 60051 | 306035 (5) | 578305 (10) | 591953 (10) |
| AU 101 (IFNg) | 85816 | 473395 (6) | 724129 (8) | 817447 (10) |
| AU 5 (EDN1) | 69391 | 613921 (9) | 838040 (12) | 1023000 (15) |
| AU 3 (CD247) | 44939 | 595753 (13) | 961839 (21) | 1116000 (25) |
| AU 20 | 1135000 | 10750000 (9) | 21020000 (19) | 25480000 (22) |
| (TMEM- | | | | |
| 219snp) | | | | |
| AU 10 (Myc) | 1233000 | 16020000 (13) | 26780000 (22) | 27800000 (23) |
| AU 7 (SLC2A1) | 4914 | 80906 (16) | 132974 (27) | 136537 (28) |
| AU Construct | Day 1 | Day 3 | Day 6 | Day 8 |
| AU 21 (CCR7) | 27128 | 465140 (17) | 604016 (22) | 692715 (26) |
| AU 23 | 71105 | 1215000 (17) | 2012000 (28) | 2110000 (30) |
| (CDC42-SE2) | | | | |
| AU 22 | 226815 | 2829000 (12) | 6106000 (27) | 7396000 (33) |
| (SEM-A4D) | | | | |
| AU 19 | 833146 | 11260000 (14) | 22560000 (27) | 27500000 (33) |
| (TMEM-219) | | | | |

The AU elements in FIG. 6, FIG. 7 and Table 1 had different basal expression levels, different induced expression levels (at 24 hours), and different levels of fold induction. The basal expression levels differed over an about 2000 fold range for these AU elements (AU 7 to AU 20), and the induced expression levels differed over an about 5500 fold range (AU 7 to AU 10). Basal expression for the constructs ranged from 1390 for AU 7 (SLC2A1) to 2,927,000 for AU 20 (TMEM-219snp). Activated expression ranged from 4914 for AU 7 (day 1) to 27,800,0000 forAU 10 (day 8). FIG. 8 and Table 1 show that some AU elements had lower levels ofoutput, for example, AU 101 (IFNg), AU 2 (CSF2), AU 5 (EDN1), AU 7 (SLC2A1), AU 21 (CCR7), and AU 23 (CDC42-SE2). Some AU elements had intermediate amounts of output: AU 19 (TMEM-219) and AU 22 (SEM-A4D). And some AU element had high output: AU 20 (TMEM-219snp) and AU 10 (Myc).

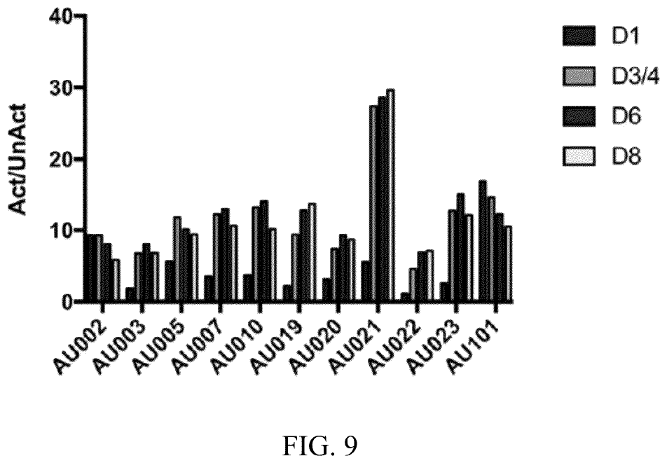

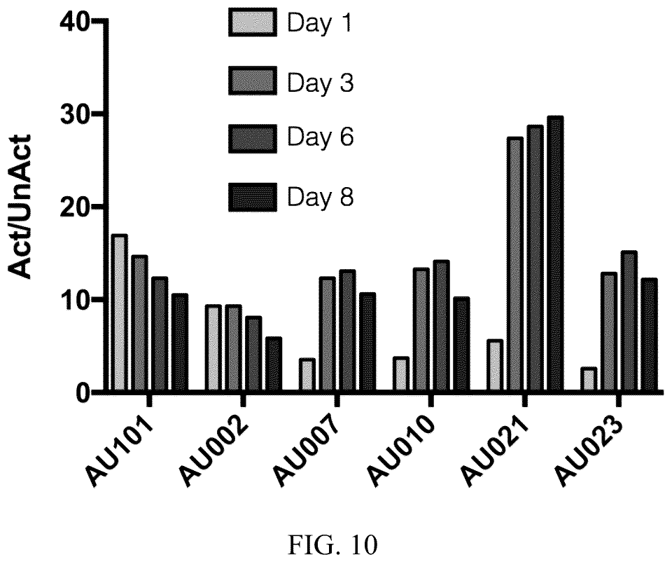

The Luciferase data was also analyzed for dynamic range (fold induction or luciferase activated/luciferase basal) of each luciferase-AU construct. The dynamic range (fold induction) for each AU construct at Days 1, 3/4 (activated expression was measured on Day 3 and basal expression was measured on Day 4), 6 and 8. This data is shown below in Table 2, and plotted in bar graphs in FIG. 9 and FIG. 10.

TABLE 2
Fold Induction

| AU Construct | Day 1 | Day 3/4* | Day 6 | Day 8 |
|---|---|---|---|---|
| AU 2 | 9.3 | 9.3 | 8.1 | 5.8 |
| AU 101 | 16.9 | 14.6 | 12.3 | 10.5 |
| AU 5 | 5.7 | 11.9 | 10.1 | 9.4 |
| AU 21 | 5.6 | 27.3 | 28.6 | 29.6 |
| AU 3 | 1.8 | 6.7 | 8.1 | 6.8 |
| AU 20 | 3.1 | 7.3 | 9.3 | 8.7 |
| AU 10 | 3.7 | 13.2 | 14.1 | 10.2 |
| AU 7 | 3.5 | 12.3 | 13.1 | 10.6 |
| AU 23 | 2.6 | 12.8 | 15.1 | 12.2 |
| AU 19 | 2.3 | 9.3 | 12.9 | 13.8 |
| AU 22 | 1.2 | 4.6 | 6.9 | 7.1 |

*Induction was measured on Day 3 and basal was measured on Day 4.

At Day 1 dynamic range (fold induction=activated/basal) ranged from about 1 (AU 22) to about 17 (AU 101). At Day 3/4, dynamic range varied from about 4.5 (AU 22) to about 27 (AU21). At Day 6, dynamic range varies from about 7 (AU 22) to about 29 (AU 21). On Day 8, dynamic range varied from about 7 (AU22) to about 30 (AU 21). The AU constructs showed a number of related patterns. AU 2 and AU 101 showed a rapid increase in dynamic range on Day 1, and then the dynamic range decreased on days 6 and 8. AU 5 and AU 21 show increasing dynamic range from day 0 to day 3/4, and then the dynamic range is maintained through days 6 and 8. AU 3, AU 20, AU 10, AU 7 and AU 23 showed rising dynamic range from day 0 to day 6, and then the dynamic range decreased on day 8. AU 19, and AU 22, showed rising dynamic ranges from day 0 to day 8.

AU 21 and AU 23 showed accelerating dynamic range and these AU constructs also had low basal expression (day 1=4865 and 27363, respectively). AU 2 and AU 101 showed decreasing dynamic range from 24 hours to 72 hours and these AU elements also had low basal expression. AU 5 and AU 20 also showed decreasing dynamic range from day 1 to day 3/4 (though more expression than AU 2 and AU 101) and AU 5 had low basal expression whereas AU 20 had high basal expression. AU 10, AU 19 and AU 22 showed consistent dynamic range from day 1 to day 3/4 and had high basal levels of expression. AU 3 and AU 7 also had consistent dynamic range from day 1 to day 3/4 and had low basal expression levels.

The above data shows that different AU elements have different temporal effects on expression from days 1-8. Some AU elements show accelerating dynamic range over different portions of the time range. The AU elements show different amounts of total expression (Cₘₐₓ) and different times to maximum expression (Tₘₐₓ). The AU elements also show different maximum dynamic ranges and time to reach these maximums. These differing kinetics of expression can be used to provide customized basal, Cₘₐₓ, Tₘₐₓ, dynamic range, and time to max dynamic range for a desired transgene. These differing kinetics can also be used to provide temporally distinct expression for two transgenes in a cell after activation of the cell.

### Example 12: AU Element Control with Glucose and Galactose

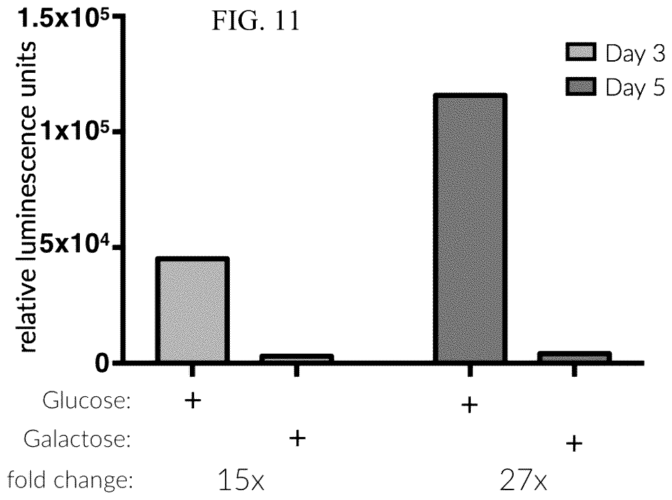

Constructs were made with different RDEs operably linked to a nucleic acid encoding luciferase. The RDE was an AU element responsive to glycolytic state of the cell. The AU element—luciferase constructs were transduced into T-cells. After the cells reached the resting state, they were split into wells and fed media including either glucose or galactose. Luciferase activity was measured on days 3 and 5. These results are shown in the bar graph of FIG. 11. The results show that glucose increased expression of luciferase compared to galactose and the amount of expression increased from days 3 to 5. On day 3 the glucose treated cells had 15× more expression of luciferase than the galactose treated cells and on day 5 this had grown to 27× more expression.

### Example 13: Payload Delivery Using Gold in a Mouse Lymphoma Model

Constructs were made using an anti-CD19 CAR cassette as described in WO 2012/079000, which is hereby incorporated-by-reference in its entirety for all purposes, and a Luciferase-AU (3′ UTR of IL-6) insert. These constructs were placed in a bicistronic lenti virus construct. The anti-CD19 CAR cassette and the insert with the luciferase-RDE are transcribed in opposite directions on the bicistronic vector, and the control regions for each are located in between the two insert/cassettes. The control region for the Luciferase-RDE insert was a MinP promoter. The control region of the anti-CD19 CAR cassette was the MND promoter. CD4+ T-cells were transduced with the bicistronic construct.

A second construct was made using the anti-CD19 CAR cassette described above and a Luciferase insert (without the RDE element so that expression was constitutive). Both constructs were separately transduced into different groups of T-cells.

The transduced T cells were allowed to return to resting state, and then were tested after stimulation as follows. For the ‘no stimulation’ set, transduced T-cells were incubated for 24 h alone in medium. For the ‘Raji co-culture’ set CD19+ Raji B cells were incubated with the transduced T cells for 24 h. At 24 h, the T cells were stained for CD25 and CD69, which are activation markers, and subject to flow cytometry to measure these markers and luciferase expression in the T cells. These in vitro results showed that the anti-CD19 CAR T-cells made luciferase after activation of the T-cells through the CAR.

These anti-CD19 CAR T-cells with the luciferase-RDE were also tested in a mouse model for lymphoma. CD19+ Raji cells were implanted in the flanks of NSG mice. After tumor formation, the anti-CD19 CAR T-cells were injected into the mice and the mice were scanned for luminescence. Imaging of the mice showed luminescence at the tumor sites from anti-CD19 CAR T-cells that have been activated by the CD19 positive tumor. The amount of luminescence increased overtime as more T-cells were activated. In contrast, the anti-CD19 CAR T-cells with constitutive expression of luciferase should luminescence throughout the mice as well as at the site of the tumors in the flanks of the mice.

### Example 15: Payload Delivery to αvβ6 Positive Solid Tumor

A nucleic acid encoding a knottin as described in Silverman et al., J. Mol. Biol. 385:1064-75 (2009) and Kimura et al, Proteins 77:359-69 (2009), which are incorporated by reference in their entirety for all purposes is operably linked to a nucleic acid encoding the CAR components aCD43z,CD8Hinge,CD8transmembrane,41BB(CD28 or other costim), and CD3z to make a nucleic acid encoding an anti-avj36 CAR.

The nucleic acid encoding the anti-avj36 CAR is transfected by routine methods into T-cells (Jurkat cells and/or primary human T-cells), and stable populations of T-cells are selected using appropriate antibiotics (or other selection schemes). T-cell populations with anti-av36 CARs are activated by co-incubation with anti-CD3/CD28 beads. These cells are also engineered with an expression cassette encoding IL-12 operably linked to the Gold element from INFg or AU 21 (CCR7) is placed under the control of the promoter Min P.

The anti-αvβ6 CAR T-cells are incubated in wells with αvβ6 tumor cells. After incubation, the wells are tested for secretion of IL-12 from the anti-αvβ6 CAR T-cells. anti-αvβ6 CAR T-cells secrete IL-12 when incubated with αvβ6 tumor cells, and the controls show low or no secretion when the CAR T-cell is not stimulated.

### Example 14: An Anti-Onco CD 43 CAR for AML

A single chain antibody for onco-sialylated CD 43 was made using an anti-onco-sialylated CD 43 antibody. The nucleic acid encoding this single-chain antibody was combined with a nucleic acid encoding the CAR components aCD43z,CD8Hinge,CD8transmembrane,41BB(CD28 or other costim), and CD3z to make a nucleic acid encoding an anti-onco-sialylated CD 43 CAR.

The nucleic acid encoding the anti-onco-sialylated CD 43 CAR is transfected by routine methods into T-cells (Jurkat cells and/or primary human T-cells), and stable populations of T-cells are selected using appropriate antibiotics (or other selection schemes). T-cell populations with anti-onco-sialylated CD 43 CARs are activated by co-incubation with anti-CD3/CD28 beads.

### Example 15: Payload Delivery to CD 43 Positive AML

An expression cassette encoding IL-12 operably linked to the Gold element from INFg or AU 21 (CCR7) is placed under the control of the promoter Min P, and engineered into the anti-onco-sialylated CD 43 CAR T-cell.

The anti-onco-sialylated CD 43 CAR T-cells are incubated in wells with AML cells. After incubation, the wells are tested for secretion of IL-12 from the anti-onco-sialylated CD 43 CAR T-cells. Anti-onco-sialylated CD 43 CAR T-cells secrete IL-12 when incubated with AML cells, and the controls show low or no secretion when the CAR T-cell is not stimulated.

### Example 16: miRNA as a Payload

A payload transgene encoding IL-12 is engineered to have an artificial intron encoding a mir155 cassette as disclosed in Du et al., FEBs Journal 273:5421-5427 (2006) or Chung et al., Nucl Acids Res 34:e53 (2006). The mir155 cassette is engineered to include an AU element such as, for example, AU101 (IFNg) or AU 14 (IL-6), operably linked to it, and the transgene is also engineered with an AU element such as AU101 or AU14. This transgene with the mir155 intron is engineered into primary T-cells. An anti-CD19 CAR as described in Example 14 is also engineered into the primary T-cells.

The anti-CD19 CAR T cells with the IL-12 payload are allowed to return to resting state, and then are tested after stimulation as follows. For the ‘no stimulation’ set, transduced T-cells are incubated for 24 h alone in medium. For the ‘Raji co-culture’ set CD19+ Raji B cells are incubated with the transduced T cells for 24 h. At 24 h, the T cells are stained for CD25 and CD69, which are activation markers, and subject to flow cytometry to measure these markers. The cells are also tested for expression of the payload IL-12.

These anti-CD19 CAR T-cells with the IL-12 payload are also tested in a mouse model for lymphoma. CD19+ Raji cells are implanted in the flanks of NSG mice. After tumor formation, the anti-CD19 CAR T-cells with the IL-12 payload are injected into the mice. At every third day starting at day 4 after administration, tumor killing in the mice is measured using calipers.

### Example 17: IL-12 Payload Delivery

A construct with an anti-CD19 CAR as described in Example 14 was made. A construct with the NFAT promoter operably linked to a nucleic acid encoding IL-12 followed by AU101 (the RDE from INFg) was also made. The IL-12 transcript made from the construct operably links the coding sequence for IL-12 to the AU101 RDE. A second IL-12 construct was made that provided constitutive expression of IL-12. A third construct placed Luciferase under control of an AU14 (IL-6).

The constructs were transduced into primary T-cells which were then allowed to return to a resting state. This produced anti-CD19 CAR T-cells with payloads of IL-12 (RDE controlled or constitutive) or luciferase.

The primary T-cells with the anti-CD19 CAR and IL-12 payload (RDE controlled or constitutive) or luciferase payload were administered to mice bearing CD19+ tumors in their flanks. Killing of tumor cells was monitored over 42 days. The mice which received T-cells with the anti-CD19 CAR and luciferase payload showed a moderate amount of tumor cell killing (about 3 logs). The mice receiving the IL-12 payloads had a large amount of tumor cell killing (6-7 logs). A comparison of IL-12 serum levels in the mice receiving the constitutive or AU101 controlled IL-12 had 10-fold differences in the systemic IL-12 levels with the AU101 controlled payload having 10 times lower amounts of IL-12 than the constitutive IL-12 payload.

The RDE control of IL-12 expression lowered systemic IL-12 levels in the mice but gave localized concentrations of IL-12 that improved tumor cell killing. After the activated CAR T-cells kill the tumor cells these CAR T-cells can migrate from the tumor site to lymph nodes and/or the spleen where they can educate other T-cells and form memory T-cells.

### Example 18: Payload Delivery to DLL3+ Cancer Cells

CAR constructs are made using an anti-DLL3 antibody domain such as described in US20170137533 (which is incorporated by reference in its entirety for all purposes) as SC16.15. This anti-DLL3 antibody domain is made into a single chain antibody (scFv), and the anti-DLL3 scFv is combined with the transmembrane and intracellular portions of a CAR (such as those described in WO 2012/079000, which is hereby incorporated-by-reference in its entirety for all purposes) to make an anti-DLL3 CAR.

Payload constructs are made by engineering a transgene with an RDE so that when the transgene is transcribed the transcript for the transgene operably links the transgene to the RDE. The payload transgene can encode an anti-4-1BB antibody, an anti-CD11b antibody, an anti-CTLA4 antibody, an anti-IL1b antibody, a BiTE, a CCL2, an anti-CXCR4 antibody, an anti-CXCL12 antibody, a HAC, a heparinase, a hyaluronidase, a Hsp60, a Hsp70, an IL-2, an IL-12, an IL-15, an IL-18, an INFγ, a miRNA (e.g., mir155), a CD40 ligand, an ApoE3, an ApoE4, an antagonists of CSF1 receptor, a TNFα, and/or an anti-CD28 antibody. The RDE can be AU101 (INFg) or AU14 (IL-6).

The constructs are transduced into primary T-cells which are then allowed to return to a resting state. This produced anti-DLL3 CAR T-cells with one or more of the payloads: anti-CXCL12 antibody, anti-CXCR4 antibody, IL-12, anti-4-1BB antibody, anti-CD11b antibody, anti-CTLA4 antibody, anti-IL1b antibody, a BiTE, CCL2, HAC, heparinase, hyaluronidase, Hsp60, Hsp70, IL-2, IL-15, IL-18, INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies).

An NSG mouse model from Jackson Laboratories is used to establish cancer xenografts of human melanoma, human small cell lung cancer (SCLC), and human IDHlmut glioma. After the cancer xenograft is established in the mice, the mice are treated with the primary T-cells with the anti-DLL3 CAR and one of the payloads. Cancer xenograft killing is then compared between the different payloads of the DLL3-CAR T-cells.

### Example 19: Coordinated Delivery of CXCL9 and an Anti-PD1 Therapy to DLL3+ Cancer Cells

An anti-DLL3 CAR is made as described in Example 20. This CAR construct is engineered into T-cells also as described in Example 20.

Two payload cassettes are made for delivery by the anti-DLL3 CAR T-cell. First, a construct is made that encodes CXCL9 as a secreted payload operably linked to an RDE with an early expression profile (early maximal expression after activation of the cell) such as AU2 (CSF-2, maximal fold induction on day 1), AU101 (IFNg, maximal fold induction on day 1), or AU5 (EDN1, maximal fold induction on day 3/4). Second, a construct is made that encodes an anti-PD1 antibody (e.g., Pembrolizumab (Keytruda@)) as a secreted payload operably linked to an RDE with a late expression profile (late maximal expression after activation of the cell) such as AU22 (SEM-A4D, maximal fold induction on day 8) or AU19 (TMEM-219, maximal fold induction on day 8). The two payloads can be placed into a bicistronic construct, placed on the same construct, or the payloads can be expressed from separate constructs. The payload construct(s) are engineered into the anti-DLL3 CAR T-cell as described above in Example 20.

When this engineered CAR T-cell is administered to NSG mouse model as described in Example 20. The CAR T-cells are activated by DLL3 at the tumor target, and the RDE constructs with the CXCL9 express this payload first, and then at a later time the anti-PD1 antibody payload is expressed. The AU2, AU5 or AU101 RDE of the CXCL9 construct has an early maximal expression of about 1 day after activation of the cell by DLL3 at a cancer target. The CXCL9 can be secreted early after activation of the T-cell by DLL3 and the CXCL9 can potentiate the T-cell responses to tumors treated with anti-PD1 antibodies. After CXCL9 secretion, anti-PD1 is maximally secreted at a later time (about 8 days) and the effect of this antibody can be increased by the pretreatment with CXCL9.

The early expression of CXCL9 potentiates the activity and cancer killing from the anti-PD1 antibody.

### Example 20: Combination Therapy

CAR constructs are made using an anti-DLL3 antibody domain such as described in US20170137533 (which is incorporated by reference in its entirety for all purposes) as SC16.15 or SC16.25. This anti-DLL3 antibody domain is made into a single chain antibody (scFv), and the anti-DLL3 scFv is combined with the transmembrane and intracellular portions of a CAR (such as those described in WO 2012/079000, which is hereby incorporated-by-reference in its entirety for all purposes) to make an anti-DLL3 CAR.

Payload constructs are made by engineering a transgene with an RDE so that when the transgene is transcribed the transcript for the transgene operably links the transgene to the RDE. The payload transgene can encode an anti-4-1BB antibody, an anti-CD11b antibody, an anti-CTLA4 antibody, an anti-ILlb antibody, a BiTE, a CCL2, an anti-CXCR4 antibody, an anti-CXCL12 antibody, a HAC, a heparinase, a hyaluronidase, a Hsp60, a Hsp70, an IL-2, an IL-12, an IL-15, an IL-18, an INFγ, a miRNA (e.g., mir155), a CD40 ligand, an ApoE3, an ApoE4, an antagonists of CSF1 receptor, a TNFα, and/or an anti-CD28 antibody. The RDE can be AU101 (INFg) or AU14 (IL-6).

The constructs are transduced into primary T-cells which are then allowed to return to a resting state. This produced anti-DLL3 CAR T-cells with one or more of the payloads: anti-CXCL12 antibody, anti-CXCR4 antibody, IL-12, anti-4-1BB antibody, anti-CD1 1b antibody, anti-CTLA4 antibody, anti-IL1b antibody, a BiTE, CCL2, HAC, heparinase, hyaluronidase, Hsp60, Hsp70, IL-2, IL-15, IL-18, INFγ, miRNA (e.g., mir155), CD40 ligand, ApoE3, ApoE4, TNFα, CCR2, CCR4/CXCL12, CXCR3+CXCL9, CXCL9, ACLY, antagonists of CSF1 receptor, miRNA for Tox (e.g., hsa-mir-26b-5p (MIRT030248) hsa-mir-223-3p (MIRT054680)), miRNA for TCF-7 (e.g., mIR-192, mIR-34a, miR-133a, miR-138-5p, miR-342-5p, miR-491-5p, miR-541-3p), and/or anti-CD28 antibody (including full length and fragments such as single chain antibodies).

An antibody drug conjugate (ADC) is made between an anti-DLL3 antibody such as described in US20170137533 (which is incorporated by reference in its entirety for all purposes) as SC16.15 or SC16.25. This anti-DLL3 antibody domain is converted to an appropriate format (e.g., a Fab, F(ab′)2 or full-length IgG) and conjugated to one or more drugs (e.g., etoposide, irinotecan, cisplatin and/or carboplatin).

An NSG mouse model from Jackson Laboratories is used to establish cancer xenografts of human melanoma, human small cell lung cancer (SCLC), and human IDHlmut glioma. After the cancer xenograft is established in the mice, the mice are treated with the primary T-cells with the anti-DLL3 CAR and one of the payloads, anti-DLL3 ADC, or primary T-cells with the anti-DLL3 CAR and one of the payloads and the anti-DLL3 ADC. Cancer xenograft killing is then compared between the ADC, different payloads of the DLL3-CAR T-cells, and the different payloads of the DLL3 CAR T-cells with the anti-DLL3 ADC.

### Example 21: Notch Inhibitors Enhance T-Cell Activation

A anti-DLL3 CAR is made as described above. This anti-DLL3 CAR is placed into a primary T-cell as described above. The anti-DLL3 CAR T-cells can also include a payload under the control of an RDE.

The anti-DLL3 CAR T-cells are mixed with SHP77 cells (cancer cell line expression DLL3) at different ratios (e.g., CAR T-cell to SHP77 of 1:1, 1:3, 1:10) in the presence of absence of dibenzazepine (a gamma secretase inhibitor) at concentrations of 1 nM, 10 nM, 100 nM and 1 uM.

The anti-DLL3 CAR T-cell are also administered to a NSG mouse model as described in Example 20. These administrations are also done with or without administration of dibenzazepine.

### Example 22: Activity of Engineered IL-18 Variants

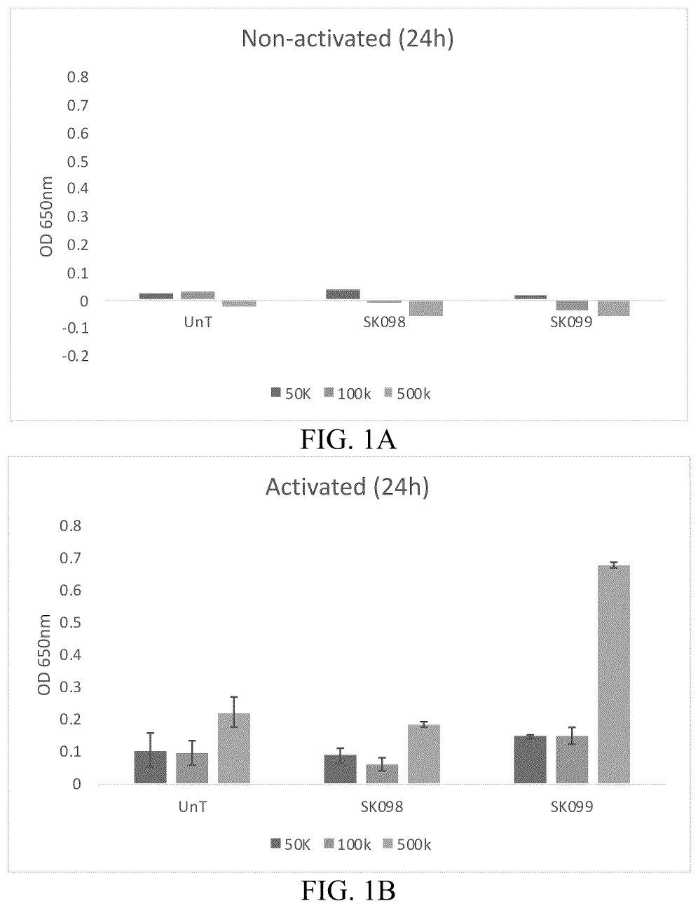

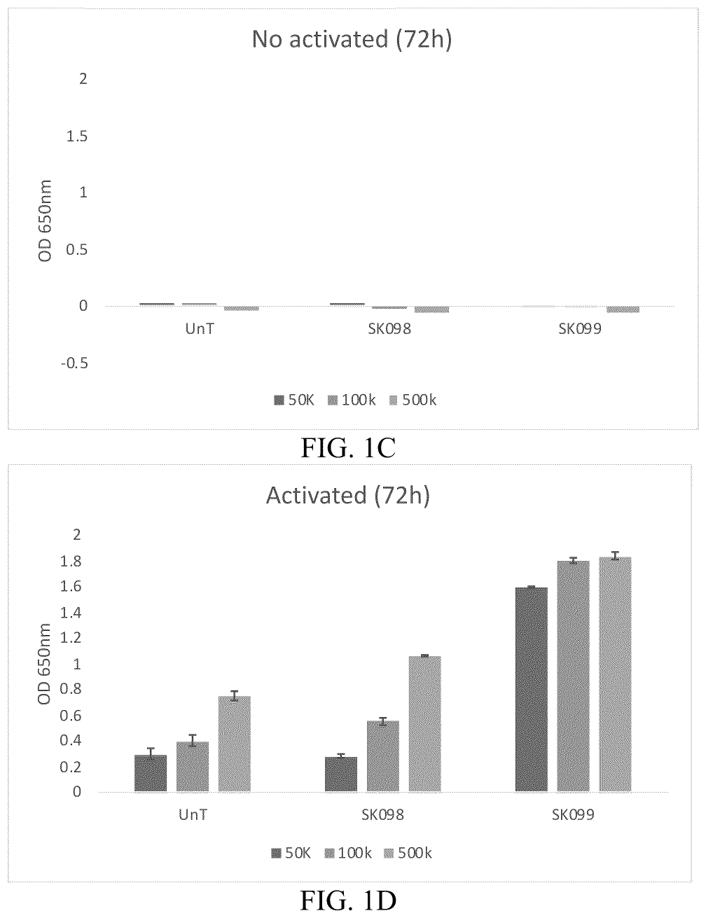

Wild-type IL-18 and an IL-18 variant (M51A, K53G, Q56R, P57A and M60K) were engineered into T-cells. Non-transduced T-cells, T-cells transduced with wild-type IL-18, and T-cells transduced with IL-18 variant were tested. The T-cells were activated with anti-CD3/CD28 beads and proliferation of the T-cells was measured at 24 hours and 72 hours. FIG. 1A and FIG. 1B show the proliferation of T-cells (nontransduced, wild-type IL-18, and IL-18 variant) at 24 hours for non-activated T-cells (FIG. 1A) and activated T-cells (FIG. 1B). FIG. 1C and FIG. 1D show the proliferation of T-cells (nontransduced, wild-type IL-18, and IL-18 variant) at 72 hours for non-activated T-cells (FIG. 1C) and activated T-cells (FIG. 1D). Each of the Figures shows the proliferation of T-cells at three different starting amounts: 50,000 T-cells, 100,000 T-cells and 500,000 TO-cells.

At 72 hours, the T-cells with the IL-18 variant showed more growth that the non-transduced T-cells and more growth than the T-cells with wild-type IL-18. The lowest amount of starting T-cells with the IL-18 variant (50,000) had more proliferation that the highest starting amount of T-cells (500,000) with wild-type IL-18. At 24 hours, the T-cells with the IL-18 variant showed more growth that the non-transduced T-cells and more growth than the T-cells with wild-type IL-18 when comparing proliferation at each amount of starting T-cells.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the scope of the invention(s) of the disclosure.

1. An engineered IL-18 polypeptide capable of increasing proliferation of activated T-cells as compared to a wild-type IL-18 comprising an amino acid sequence selected from SEQ ID NO: 3 or 4.

2. The engineered IL-18 of claim 1 wherein the amino acid sequence is that of SEQ ID NO: 3.

3. The engineered IL-18 of claim 1 wherein the amino acid sequence is that of SEQ ID NO: 4.

4. An engineered IL-18 polypeptide having at least 60%, 65%, 70%, 75%, 80%, 85%, 90%, 91%, 92%, 93%, 94%, 95%, 96%, 97%, 98%, 99% or more sequence identity to a reference polypeptide comprising an amino acid sequence selected from
(a) SEQ ID NO: 3;
(b) SEQ ID NO: 4; and
(c) SEQ ID NO: 1 and having one or more amino acid substitutions at reference residue positions corresponding to: M51, K53, Q56, P57, and M60, wherein the reference residue positions are with respect to the reference sequence of SEQ ID NO: 3.

5. A polynucleotide encoding the engineered IL-18 polypeptide of claim 1.

6. The polynucleotide of claim 5, comprising a nucleotide sequence selected from the group consisting of SEQ ID NO: 7 or 8.

7. A vector comprising the polynucleotide of claim 5.

8. The vector of claim 7, wherein the vector is an expression vector.

9. A host cell comprising a polynucleotide of claim 8.

10. The host cell of claim 9, wherein the host cell is a mammalian cell, an insect cell, a yeast cell, or a microbial cell.

11. A method for producing an engineered IL-18, comprising contacting the host cell of claim 10 under conditions suitable for synthesis of the engineered IL-18.

12. A method of controlling a transgene encoding an engineered IL-18, comprising the steps of: obtaining a eukaryotic cell comprising a receptor, and a heterologous nucleic acid comprising a polynucleotide encoding a promoter operably linked to the transgene encoding an engineered IL-18 that is operably linked to a polynucleotide encoding a RNA degradation element (RDE), wherein the RDE is an AU rich element, wherein the heterologous nucleic acid is transcribed to make a transcript encoding the transgene encoding an engineered IL-18 operably linked to the RDE; exposing the primary T-cell to a ligand for the receptor wherein binding of the ligand by the receptor activates the eukaryotic cell; and expressing the transgene encoding an engineered IL-18 wherein the amount of the engineered TL-18 made from the transgene is increased after the activation of the eukaryotic cell.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A polynucleotide encoding the engineered IL-18 polypeptide of claim 2.

28. A polynucleotide encoding the engineered IL-18 polypeptide of claim 3.

29. A vector comprising the polynucleotide of claim 27.

30. The vector of claim 29, wherein the vector is an expression vector.

31. A vector comprising the polynucleotide of claim 27.

32. The vector of claim 29, wherein the vector is an expression vector.

33. A host cell comprising a polynucleotide of claim 30.

34. A host cell comprising a polynucleotide of claim 32.
